(19) United States
(12) Patent Application Publication
(10) Pub. No.: US 2022/0144949 A1
(43) Pub. Date: May 12, 2022

(54) CONDITIONALLY ACTIVATED BINDING PROTEINS CONTAINING Fc REGIONS AND MOIETIES TARGETING TUMOR ANTIGENS

(71) Applicant: TAKEDA PHARMACEUTICAL LIMITED COMPANY, Osaka (JP)

(72) Inventors: Robert B. DUBRIDGE, Belmont, CA (US); Anand PANCHAL, San Francisco, CA (US)

(21) Appl. No.: 17/435,931

(22) PCT Filed: Mar. 5, 2020
(86) PCT No.: PCT/US2020/021276

§ 371 Date: Sep. 2, 2021

Related U.S. Application Data
(60) Provisional application No. 62/814,159, filed on Mar. 5, 2019.
(60) Provisional application No. 62/814,210, filed on Mar. 5, 2019.
(60) Provisional application No. 62/814,744, filed on Mar. 6, 2019.
(60) Provisional application No. 62/826,523, filed on Mar. 29, 2019.

(51) Int. Cl. C07K 16/28; A61P 35/00

(52) U.S. Cl. CPC C07K 16/2809; A61K 2039/505; C07K 2317/569; A61P 35/00

(57) ABSTRACT

Provided herein are compositions of conditionally activated binding proteins containing Fc regions such that the proteins target tumor antigens. Also provided are methods for coexpressing and purifying such conditionally activated binding proteins. Methods of treating cancer by administering the conditionally activated binding proteins to a patient are also described.

### CROSS-REFERENCE TO RELATED APPLICATIONS

The application claims priority to U.S. Provisional Application No. 62/814,459 filed Mar. 5, 2019, U.S. Provisional Application No. 62/814,744 filed Mar. 6, 2019, U.S. Provisional Application No. 62/814,744 filed Mar. 6, 2019, and U.S. Provisional Application No. 62/826,523 filed Mar. 29, 2019, the contents are hereby incorporated by reference in its entirety.

### REFERENCE TO A “SEQUENCE LISTING,” A TABLE, OR A COMPUTER PROGRAM LISTING APPENDIX SUBMITTED ON A COMPACT DISK

The sequence listing contained in the file named “118459-5008-WO sequence listing_ST25.txt” and having a size of 101 kilobytes, has been submitted electronically herewith via EFS-Web, and the contents of the txt file are hereby incorporated by reference in their entirety.

### BACKGROUND OF THE INVENTION

The selective destruction of an individual cell or a specific cell type is often desirable in a variety of clinical settings. For example, it is a primary goal of cancer therapy to specifically destroy tumor cells, while leaving healthy cells and tissues as intact and undamaged as possible. One such method is by inducing an immune response against the tumor, to make immune effector cells such as natural killer (NK) cells or cytotoxic T lymphocytes (CTLs) attack and destroy tumor cells.

The use of intact monoclonal antibodies (MAb), which provide superior binding specificity and affinity for a tumor-associated antigen, have been successfully applied in the area of cancer treatment and diagnosis. However, the large size of intact MAbs, their poor bio-distribution and long persistence in the blood pool have limited their clinical applications. For example, intact antibodies can exhibit specific accumulation within the tumor area. In biodistribution studies, an inhomogeneous antibody distribution with primary accumulation in the peripheral regions is noted when precisely investigating the tumor. Due to tumor necrosis, inhomogeneous antigen distribution and increased interstitial tissue pressure, it is not possible to reach central portions of the tumor with intact antibody constructs. In contrast, smaller antibody fragments show rapid tumor localization, penetrate deeper into the tumor, and also, are removed relatively rapidly from the bloodstream.

Single chain fragments (scFv) derived from the small binding domain of the parent MAb offer better biodistribution than intact MAbs for clinical application and can target tumor cells more efficiently. Single chain fragments can be efficiently engineered from bacteria, however, most engineered scFv have a monovalent structure and show decreased tumor accumulation e.g., a short residence time on a tumor cell, and specificity as compared to their parent MAb due to the lack of avidity that bivalent compounds experience.

Despite the favorable properties of scFv, certain features hamper their full clinical deployment in cancer chemotherapy. Of particular note is their cross-reactivity between diseased and healthy tissue due to the targeting of these agents to cell surface receptors common to both diseased and healthy tissue. ScFvs with an improved therapeutic index would offer a significant advance in the clinical utility of these agents. The present invention provides such improved scFvs and methods of manufacturing and using the same. The improved scFvs of the invention have the unexpected benefit of overcoming the lack of avidity demonstrated by a single unit by forming a dimeric compound.

### SUMMARY OF THE INVENTION

In one aspect, provided herein is a homodimeric protein composition comprising:
(a) two monomers each comprising, from N- to C-terminal:
   i) a first single domain antigen binding domain (sdABD) that binds to a first tumor target antigen (TTA) (sdABD-TTA);
   ii) an optional domain linker;
   iii) a constrained Fv domain comprising:
      1) a variable heavy domain comprising vhCDR1, vhCDR2, and vhCDR3;
      2) a constrained, non-cleavable linker (CNCL); and
      3) a variable light domain comprising vlCDR1, vlCDR2, and vlCDR3;
   iv) an optional domain linker;
   v) a second sdABD-TTA;
   vi) a cleavable linker;
   vii) a pseudo Fv domain comprising:
      1) a pseudo variable light domain;
      2) a non-cleavable linker; and
      3) a pseudo variable heavy domain; and
   viii) an optional cleavable linker; and
   ix) an Fc domain;

wherein the variable heavy domain and first variable light domain are capable of binding human CD3 but the constrained Fv domain does not bind CD3; wherein the variable heavy domain and the pseudo variable light domain intermolecularly associate to form an inactive Fv; and wherein the variable light domain and the pseudo variable heavy domain intermolecularly associate to form an inactive Fv.

In some embodiments of the homodimeric Fc protein, the first variable heavy domain is N-terminal to the first variable light domain and the pseudo variable light domain is N-terminal to the pseudo variable heavy variable domain. In some embodiments, the first variable light domain is N-terminal to the first variable heavy domain and the pseudo variable light domain is N-terminal to the pseudo variable heavy domain. In some embodiments, the first variable light domain is N-terminal to the first variable heavy domain and the pseudo variable heavy domain is N-terminal to the pseudo variable light domain. In some embodiments, the first variable heavy domain is N-terminal to the first variable light domain and the pseudo variable heavy domain is N-terminal to the pseudo variable light domain.

In some embodiments, the CD3 variable heavy chain (CD3 VH or αCD3 VH) comprises the amino acid sequence of SEQ ID NO:186 and the CD3 variable light domain (CD3 VL or αCD3 VL) comprises the amino acid sequence of SEQ ID NO:170. In some embodiments, the CD3 variable heavy chain comprises a CDR1, CDR2, and CDR3 set forth in SEQ ID NO:186 and FIG. 2B. In some embodiments, the CD3 variable heavy chain comprises a CDR1, CDR2, and CDR3 as set forth in SEQ ID NOS:187-189, respectively. In some embodiments, the CD3 variable light chain comprises a CDR1, CDR2, and CDR3 set forth in SEQ ID NO:170 and FIG. 2A. In some embodiments, the CD3 variable heavy chain comprises a CDR1, CDR2, and CDR3 as set forth in SEQ ID NOS:171-173, respectively.

In some embodiments, the pseudo variable heavy domain (CD3 VHi or αCD3 VHi) comprises the amino acid sequence of SEQ ID NO:190 and the pseudo variable light domain (CD3 VLi) comprises the amino acid sequence of SEQ ID NO:174. In some embodiments, CD3 VHi comprises a CDR1, CDR2, and CDR3 set forth in SEQ ID NO:190 and FIG. 2B. In some embodiments, CD3 VHi comprises a CDR1, CDR2, and CDR3 set forth in SEQ ID NOS:191-193, respectively and FIG. 2B. In some embodiments, CD3 VLi comprises a CDR1, CDR2, and CDR3 set forth in SEQ ID NO:174 and FIG. 2A. In some embodiments, CD3 VLi comprises a CDR1, CDR2, and CDR3 set forth in SEQ ID NOS:175-177, respectively and FIG. 2A.

In some embodiments, the pseudo variable heavy domain (CD3 VHi2 or αCD3 VHi2) comprises the amino acid sequence of SEQ ID NO:194 and the pseudo variable light domain (CD3 VLi2 or αCD3 VLi2) comprises the amino acid sequence of SEQ ID NO:178. In some embodiments, CD3 VHi2 comprises a CDR1, CDR2, and CDR3 set forth in SEQ ID NO:194 and FIG. 2B. In some embodiments, CD3 VHi comprises a CDR1, CDR2, and CDR3 set forth in SEQ ID NOS: 195-197, respectively and FIG. 2B. In some embodiments, CD3 VLi2 comprises a CDR1, CDR2, and CDR3 set forth in SEQ ID NO:178 and FIG. 2A. In some embodiments, CD3 VLi2 comprises a CDR1, CDR2, and CDR3 set forth in SEQ ID NOS:179-181, respectively and FIG. 2A.

In some embodiments, the pseudo variable heavy domain (CD3 VHiGL4 or αCD3 VHiGL4) comprises the amino acid sequence of SEQ ID NO:198 and the pseudo variable light domain (CD3 VLiGL or αCD3 VLiGL) comprises the amino acid sequence of SEQ ID NO:182. In some embodiments, CD3 VHiGL4 comprises a CDR1, CDR2, and CDR3 set forth in SEQ ID NO:198 and FIG. 2B. In some embodiments, CD3 VHiGL4 comprises a CDR1, CDR2, and CDR3 set forth in SEQ ID NOS:199-201, respectively and FIG. 2B.

In some embodiments, CD3 VLiGL comprises a CDR1, CDR2, and CDR3 set forth in SEQ ID NO:182 and FIG. 2A. In some embodiments, CD3 VLiGL comprises a CDR1, CDR2, and CDR3 set forth in SEQ ID NOS:183-185, respectively and FIG. 2A.

In some embodiments, the TTA is selected from the group consisting of EGFR, FOLR1, B7H3, EpCAM, Trop2, and CA9.

In certain embodiments, the first and second sdABDs bind to the same TTA. In some embodiments, the first and second sdABDs bind to different TTAs. In some embodiments, the first and second sdABD-TTAs are the same. In some embodiments, the first and second sdABD-TTAs are different.

In some embodiments, an sdABD that binds to a specific TTA is selected from the group consisting of SEQ ID NOS:50, 54, 58, 62, 66, 70, 74, 78, 82, 86, 90, 94, 98, 102, 16, 110, 114, 118, 122, 126, 130, 134, 138, 142, 146, 151, 154, 158, 162, and 166. In some embodiments, the sdABD comprises a CDR1, CDR2, and CDR3 as set forth in SEQ ID NOS:50, 54, 58, 62, 66, 70, 74, 78, 82, 86, 90, 94, 98, 102, 16, 110, 114, 118, 122, 126, 130, 134, 138, 142, 146, 151, 154, 158, 162, and 166. In some embodiments, the sdABD comprises a CDR1, CDR2, and CDR3 as set forth in FIG. 1.

In some embodiments, the first cleavable linker and/or the optional cleavable linker are cleaved by a human protease selected from the group consisting of MMP2, MMP9, meprin, Cathepsin S, Cathepsin K, Cathespin L, GranzymeB, uPA, Kallekriein7, matriptase and thrombin. In some embodiments, the cleavable linker comprises an amino acid sequence depicted in FIGS. 3A-3D. In certain embodiments, the cleavable linker comprises an amino acid sequence selected from the group consisting of SEQ ID NOS:210-281. In some embodiments, the optional cleavable linker comprises an amino acid sequence depicted in FIGS. 3A-3D. In certain embodiments, the optional cleavable linker comprises an amino acid sequence selected from the group consisting of SEQ ID NOS:210-281.

In some embodiments, the homodimeric Fc protein comprises two monomers each comprising an sdABD, a CD3 variable heavy domain, a CD3 variable light domain, a CD3 pseudo variable heavy domain, a CD3 pseudo variable light domain, a cleavable linker, and an Fc domain. In some embodiments, the sdABD binds to a TTA selected from the group consisting of EGFR, FOLR1, B7H3, EpCAM, Trop2, and CA9. In some embodiments, the sdABD comprises a sequence selected from the group consisting of SEQ ID NOS:50, 54, 58, 62, 66, 70, 74, 78, 82, 86, 90, 94, 98, 102, 16, 110, 114, 118, 122, 126, 130, 134, 138, 142, 146, 151, 154, 158, 162, and 166. In some embodiments, the homodimeric Fc protein comprises two monomers each comprising an sdABD targeting EGFR, FOLR1, B7H3, EpCAM, Trop2, or CA9, a CD3 variable heavy domain, a CD3 variable light domain, a CD3 pseudo variable heavy domain, a CD3 pseudo variable light domain, a cleavable linker, and an Fc domain.

In some embodiments, each monomer of the homodimeric Fc protein comprises an amino acid sequence selected from the group consisting of Pro556 (SEQ ID NO:36), Pro587 (SEQ ID NO:38), Pro588 (SEQ ID NO:39), and Pro589 (SEQ ID NO:40). In some embodiments, the homodimeric Fc protein comprises two monomers each comprising an EGFR ABD, a CD3 variable heavy domain, a CD3 variable light domain, a CD3 pseudo variable heavy domain, a CD3 pseudo variable light domain, a cleavable linker, and an Fc domain. In some embodiments, each monomer comprises from N-terminus to C-terminus: an EGFR sdABD, a CD3 variable heavy domain, a CD3 variable light domain, an EGFR sdABD, a cleavable linker, a CD3 pseudo variable light domain, a CD3 pseudo variable heavy domain, and an Fc domain. In some embodiments, each monomer comprises from N-terminus to C-terminus: an EGFR sdABD, a CD3 variable light domain, a CD3 variable heavy domain, an EGFR sdABD, a cleavable linker, a CD3 pseudo variable light domain, a CD3 pseudo variable heavy domain, and an Fc domain. In some embodiments, each monomer comprises from N-terminus to C-terminus: an EGFR sdABD, a CD3 variable heavy domain, a CD3 variable light domain, an EGFR sdABD, a cleavable linker, a CD3 pseudo variable heavy domain, a CD3 pseudo variable light domain, and an Fc domain. In some embodiments, each monomer comprises from N-terminus to C-terminus: an EGFR sdABD, a CD3 variable light domain, a CD3 variable heavy domain, an EGFR sdABD, a cleavable linker, a CD3 pseudo variable heavy domain, a CD3 pseudo variable light domain, and an Fc domain.

In some embodiments, each monomer of the homodimeric Fc protein comprises an amino acid sequence selected from the group consisting of Pro557 (SEQ ID NO:37). In some embodiments, the homodimeric Fc protein comprises two monomers each comprising an EGFR sdABD, a CD3 variable heavy domain, a CD3 variable light domain, a CD3 pseudo variable heavy domain, a CD3 pseudo variable light domain, a noncleavable linker, and an Fc domain.

Another exemplary format of a heterodimeric Fc protein is provided in FIGS. 18A-18C. In another aspect, provided herein is a heterodimeric protein composition comprising:
(a) a first Fc monomer comprising a first Fc domain; and
(b) a second Fc monomer comprising, from N- to C terminal:
   i) a first single domain antigen binding domain (sdABD) that binds to a first tumor target antigen (TTA) (sdABD-TTA);
   ii) an optional domain linker;
   iii) a constrained Fv domain comprising:
      1) a variable heavy domain comprising vhCDR1, vhCDR2, and vhCDR3;
      2) a constrained, non-cleavable linker (CNCL); and
      3) a variable light domain comprising vlCDR1, vlCDR2, and vlCDR3;
   iv) an optional domain linker;
   v) a second sdABD-TTA;
   vi) a first cleavable linker;
   vii) a pseudo Fv domain comprising:
      1) a pseudo variable light domain;
      2) a non-cleavable linker; and
      3) a pseudo variable heavy domain; and
   viii) an optional second cleavable linker; and
   ix) a second Fc domain;
wherein said first Fc domain and said second Fc domain comprise a knob-in hole modification; wherein said variable heavy domain and said variable light domain are capable of binding human CD3 but said constrained Fv domains do not bind CD3; wherein said variable heavy domain and said pseudo variable light domain intermolecularly associate to form an inactive Fv; and wherein said variable light domain and said pseudo variable heavy domain intermolecularly associate to form an inactive Fv.

In some embodiments, the variable heavy domain is N-terminal to the variable light domain and the pseudo variable light domain is N-terminal to the pseudo variable heavy variable domain. In some embodiments, the variable light domain is N-terminal to the variable heavy domain and the pseudo variable light domain is N-terminal to the pseudo variable heavy domain. In some embodiments, the variable light domain is N-terminal to the variable heavy domain and the pseudo variable heavy domain is N-terminal to the pseudo variable light domain. In some embodiments, the variable heavy domain is N-terminal to the variable light domain and the pseudo variable heavy domain is N-terminal to the pseudo variable light domain.

In some embodiments, the CD3 variable heavy chain (CD3 VH or αCD3 VH) comprises the amino acid sequence of SEQ ID NO:186 and the CD3 variable light domain (CD3 VL or αCD3 VL) comprises the amino acid sequence of SEQ ID NO:170. In some embodiments, the CD3 variable heavy chain comprises a CDR1, CDR2, and CDR3 set forth in SEQ ID NO:186 and FIG. 2B. In some embodiments, the CD3 variable heavy chain comprises a CDR1, CDR2, and CDR3 as set forth in SEQ ID NOS:187-189, respectively. In some embodiments, the CD3 variable light chain comprises a CDR1, CDR2, and CDR3 set forth in SEQ ID NO:170 and FIG. 2A. In some embodiments, the CD3 variable heavy chain comprises a CDR1, CDR2, and CDR3 as set forth in SEQ ID NOS:171-173, respectively.

In some embodiments, the pseudo variable heavy domain (CD3 VHi or αCD3 VHi) comprises the amino acid sequence of SEQ ID NO:190 and the pseudo variable light domain (CD3 VLi) comprises the amino acid sequence of SEQ ID NO:174. In some embodiments, CD3 VHi comprises a CDR1, CDR2, and CDR3 set forth in SEQ ID NO:190 and FIG. 2B. In some embodiments, CD3 VHi comprises a CDR1, CDR2, and CDR3 set forth in SEQ ID NOS:191-193, respectively and FIG. 2B. In some embodiments, CD3 VLi comprises a CDR1, CDR2, and CDR3 set forth in SEQ ID NO:174 and FIG. 2A. In some embodiments, CD3 VLi comprises a CDR1, CDR2, and CDR3 set forth in SEQ ID NOS:175-177, respectively and FIG. 2A.

In some embodiments, the pseudo variable heavy domain (CD3 VHi2 or αCD3 VHi2) comprises the amino acid sequence of SEQ ID NO:194 and the pseudo variable light domain (CD3 VLi2 or αCD3 VLi2) comprises the amino acid sequence of SEQ ID NO:178. In some embodiments, CD3 VHi2 comprises a CDR1, CDR2, and CDR3 set forth in SEQ ID NO:194 and FIG. 2B. In some embodiments, CD3 VHi comprises a CDR1, CDR2, and CDR3 set forth in SEQ ID NOS: 195-197, respectively and FIG. 2B. In some embodiments, CD3 VLi2 comprises a CDR1, CDR2, and CDR3 set forth in SEQ ID NO:178 and FIG. 2A. In some embodiments, CD3 VLi2 comprises a CDR1, CDR2, and CDR3 set forth in SEQ ID NOS:179-181, respectively and FIG. 2A.

In some embodiments, the pseudo variable heavy domain (CD3 VHiGL4 or αCD3 VHiGL4) comprises the amino acid sequence of SEQ ID NO:198 and the pseudo variable light domain (CD3 VLiGL or αCD3 VLiGL) comprises the amino acid sequence of SEQ ID NO:182. In some embodiments, CD3 VHiGL4 comprises a CDR1, CDR2, and CDR3 set forth in SEQ ID NO:198 and FIG. 2B. In some embodiments, CD3 VHiGL4 comprises a CDR1, CDR2, and CDR3 set forth in SEQ ID NOS:199-201, respectively and FIG. 2B.

In some embodiments, CD3 VLiGL comprises a CDR1, CDR2, and CDR3 set forth in SEQ ID NO:182 and FIG. 2A. In some embodiments, CD3 VLiGL comprises a CDR1, CDR2, and CDR3 set forth in SEQ ID NOS:183-185, respectively and FIG. 2A.

In some embodiments, the TTA is selected from the group consisting of EGFR, FOLR1, B7H3, EpCAM, Trop2, and CA9.

In some embodiments, the first and second sdABDs bind to the same TTA. In some embodiments, the first and second sdABDs bind to different TTAs. In some embodiments, the first and second sdABD-TTAs are the same. In some embodiments, the first and second sdABD-TTAs are different.

In some embodiments, the sdABD(s) (e.g., the first sdABD-TTA and/or the second ABD-TTA) is selected from the group consisting of SEQ ID NOS:50, 54, 58, 62, 66, 70, 74, 78, 82, 86, 90, 94, 98, 102, 16, 110, 114, 118, 122, 126, 130, 134, 138, 142, 146, 151, 154, 158, 162, and 166.

In some embodiments, the first cleavable linker and/or the optional cleavable linker are cleaved by a human protease selected from the group consisting of MMP2, MMP9, meprin, Cathepsin S, Cathepsin K, Cathespin L, GranzymeB, uPA, Kallekriein7, matriptase and thrombin. In some embodiments, the cleavable linker comprises an amino acid sequence depicted in FIGS. 3A-3D. In certain embodiments, the cleavable linker comprises an amino acid sequence selected from the group consisting of SEQ ID NOS:210-281. In some embodiments, the optional cleavable linker comprises an amino acid sequence depicted in FIGS. 3A-3D. In certain embodiments, the optional cleavable linker comprises an amino acid sequence selected from the group consisting of SEQ ID NOS:210-281.

In some embodiments, the first Fc monomer comprises an empty Fc domain comprising a CH3-hole. In some embodiments, the first Fc monomer comprises an empty Fc domain comprising a CH3-knob. In some embodiments, the first Fc monomer comprises a hinge, CH2 domain and CH3 domain. In some embodiments, the first Fc monomer comprises a CH2 domain and CH3 domain. In some embodiments, the C-terminus of the first monomer comprises a tag such as but not limited to a histidine tag or an streptavidin tag. In some embodiments, the second Fc monomer comprises an sdABD, a CD3 variable heavy domain, a CD3 variable light domain, a CD3 pseudo variable heavy domain, a CD3 pseudo variable light domain, and an Fc domain. In some embodiments, the second monomer comprises from N-terminus to C-terminus: a first sdABD, a CD3 variable heavy domain, a CD3 variable light domain, a second sdABD, a cleavable linker, a CD3 pseudo variable light domain, a CD3 pseudo variable heavy domain, and an Fc domain. In some embodiments, the Fc domain of the second Fc monomer comprises a hinge, CH2 domain and CH3 domain. In some embodiments, the Fc domain comprises a CH2 domain and CH3 domain. In some embodiments, the C-terminus of the second monomer comprises a tag such as but not limited to a histidine tag or an streptavidin tag.

In some embodiments, the heterodimeric Fc protein comprises a first monomer comprising an empty Fc domain and a second monomer comprises from N-terminus to C-terminus: a first sdABD, a CD3 variable heavy domain, a CD3 variable light domain, a second sdABD, a cleavable linker, a CD3 pseudo variable light domain, a CD3 pseudo variable heavy domain, and an Fc domain. In some embodiments, the heterodimeric Fc protein comprises a first monomer comprising an empty Fc domain and a second monomer comprises from N-terminus to C-terminus: a first EGFR sdABD, a CD3 variable heavy domain, a CD3 variable light domain, a second EGFR sdABD, a cleavable linker, a CD3 pseudo variable light domain, a CD3 pseudo variable heavy domain, and an Fc domain. In some embodiments, the second monomer comprises from N-terminus to C-terminus: a first EGFR sdABD, a CD3 variable heavy domain, a CD3 variable light domain, a second EGFR sdABD, a cleavable linker, a CD3 pseudo variable light domain, a CD3 pseudo variable heavy domain, and an Fc domain. In some embodiments, the second monomer comprises from N-terminus to C-terminus: a first EGFR sdABD, a CD3 variable light domain, a CD3 variable heavy domain, a second EGFR sdABD, a cleavable linker, a CD3 pseudo variable light domain, a CD3 pseudo variable heavy domain, and an Fc domain. In some embodiments, the second monomer comprises from N-terminus to C-terminus: a first EGFR sdABD, a CD3 variable light domain, a CD3 variable heavy domain, a second EGFR sdABD, a cleavable linker, a CD3 pseudo variable heavy domain, a CD3 pseudo variable light domain, and an Fc domain.

In some embodiments, the second monomer comprises from N-terminus to C-terminus: a first sdABD, a CD3 variable light domain, a CD3 variable heavy domain, a second sdABD, a cleavable linker, a CD3 pseudo variable light domain, a CD3 pseudo variable heavy domain, and an Fc domain. In some embodiments, the second monomer comprises from N-terminus to C-terminus: a first sdABD, a CD3 variable heavy domain, a CD3 variable light domain, a second sdABD, a cleavable linker, a CD3 pseudo variable heavy domain, a CD3 pseudo variable light domain, and an Fc domain. In some embodiments, the second monomer comprises from N-terminus to C-terminus: a first sdABD, a CD3 variable light domain, a CD3 variable heavy domain, a second sdABD, a cleavable linker, a CD3 pseudo variable heavy domain, a CD3 pseudo variable light domain, and an Fc domain. In some instances, the first and/or second sdABD targeting EGFR comprises any one of the sequences set forth in SEQ ID NOS:50, 54, 58, 62, and 66, as depicted in FIG. 1A. In some embodiments, the first and/or second sdABD targeting FOLR1 comprises any one of the sequences set forth in SEQ ID NOS:70, 74, and 78, as depicted in FIG. 1B. In some embodiments, the first and/or second sdABD targeting B7H3 comprises any one of the sequences set forth in SEQ ID NOS:82, 86, 90, 94, 98, 102, and 106, as depicted in FIG. 1B-FIG. 1D. In some embodiments; the first and/or second sdABD targeting EpCAM comprises any one of the sequences set forth in SEQ ID NOS:110, 114, 118, and 122, as depicted in FIG. 1D-FIG. 1E. In some embodiments, the first and/or second sdABD targeting Trop2 comprises any one of the sequences set forth in SEQ ID NOS:126, 130, 134, 138, 142, and 146, as depicted in FIG. 1E-FIG. 1F. In some embodiments, the first and/or second sdABD targeting CA9 comprises any one of the sequences set forth in SEQ ID NOS:150, 154, 158, and 162, as depicted in FIG. 1F-FIG. 1G.

In some instances; the Fc domain of the second monomer comprises a CH3-knob. In some instances, the Fc domain of the second monomer comprises a CH3-hole. In some cases, a noncleavable linker is located between the CD3 variable light and heavy domains. In some cases, a noncleavable linker is located between the CD3 pseudo variable light and heavy domains.

In some embodiments, the first Fc monomer comprises an amino acid sequence of Pro574 or SEQ ID NO:41. In some embodiments, the first Fc monomer comprises an amino acid sequence of Pro688 or SEQ ID NO:47. In some embodiments, the second Fc monomer comprises an amino acid sequence of Pro575 (SEQ ID NO:42) or Pro576 (SEQ ID NO:43) or Pro577 (which is similar to Pro576 without a cleavable linker). In some embodiments, the second Fc monomer comprises an amino acid sequence of Pro689 or SEQ ID NO:48. In some embodiments, the second Fc monomer comprises an amino acid sequence of Pro690 or SEQ ID NO:49. Exemplary schemes of such a heterodimeric Fc protein comprising an empty Fc-hole and a sdABD-Fc knob are provided in FIG. 21. In some embodiments, the heterodimeric Fc comprises Pro575 and Pro574. In some embodiments, the heterodimeric Fc comprises Pro577 and Pro574. In some embodiments, the heterodimeric Fc comprises Pro576 and Pro574.

In yet another aspect, provided herein is a heterodimeric protein composition comprising:
(a) a first Fc monomer comprising, from N- to C-terminal:
   i) a first single domain antigen binding domain (sdABD) that binds to a first tumor target antigen (TTA) (sdABD-TTA);
   ii) an optional domain linker;
   iii) a first constrained Fv domain comprising:
      1) a first variable heavy domain comprising vhCDR1, vhCDR2, and vhCDR3;
      2) a first constrained, non-cleavable linker (CNCL); and
      3) a first variable light domain comprising vlCDR1, vlCDR2, and vlCDR3;
   iv) an optional domain linker;
   v) a second sdABD-TTA;
   vi) a first cleavable linker;
   vii) a first pseudo Fv domain comprising:
      1) a first pseudo variable light domain;
      2) a non-cleavable linker; and
      3) a first pseudo variable heavy domain;
   viii) a first optional cleavable linker; and
   ix) a first Fc-hole domain; and
(b) a second Fc monomer comprising, from N- to C terminal:
   i) a third sdABD-TTA;
   ii) an optional domain linker;
   iii) a second constrained Fv domain comprising:
      1) a second variable heavy domain comprising vhCDR1, vhCDR2, and vhCDR3;
      2) a second CNCL; and
      3) a second variable light domain comprising vlCDR1, vlCDR2, and vlCDR3;
   iv) an optional domain linker;
   v) a fourth sdABD-TTA;
   vi) a second cleavable linker;
   vii) a second pseudo Fv domain comprising:
      1) a second pseudo variable light domain;
      2) a non-cleavable linker; and
      3) a second pseudo variable heavy domain;
   viii) a second optional cleavable linker; and
   ix) a second Fc-knob domain;
wherein said first variable heavy domain and said first variable light domain and said second variable heavy domain and said second variable light domain are capable of binding human CD3 but said constrained Fv domains do not bind CD3; wherein said variable heavy domains and said pseudo variable light domains intermolecularly associate to form inactive Fvs; and wherein said variable light domains and said pseudo variable heavy domains intermolecularly associate to form inactive Fvs.

In some embodiments, the first variable heavy domain is N-terminal to the first variable light domain and the pseudo variable light domain is N-terminal to the pseudo variable heavy variable domain. In some embodiments, the first variable light domain is N-terminal to the first variable heavy domain and the pseudo variable light domain is N-terminal to the pseudo variable heavy domain. In some embodiments, the first variable light domain is N-terminal to the first variable heavy domain and the pseudo variable heavy domain is N-terminal to the pseudo variable light domain. In some embodiments, the first variable heavy domain is N-terminal to the first variable light domain and the pseudo variable heavy domain is N-terminal to the pseudo variable light domain.

In some embodiments, the second variable heavy domain is N-terminal to the second variable light domain and the second pseudo variable light domain is N-terminal to the second pseudo variable heavy variable domain. In some embodiments, the second variable light domain is N-terminal to the second variable heavy domain and the second pseudo variable light domain is N-terminal to the second pseudo variable heavy domain. In some embodiments, the second variable light domain is N-terminal to the second variable heavy domain and the second pseudo variable heavy domain is N-terminal to the second pseudo variable light domain. In some embodiments, the second variable heavy domain is N-terminal to the second variable light domain and the second pseudo variable heavy domain is N-terminal to the second pseudo variable light domain.

In some embodiments, the CD3 variable heavy chain (CD3 VH or αCD3 VH) comprises the amino acid sequence of SEQ ID NO:186 and the CD3 variable light domain (CD3 VL or αCD3 VL) comprises the amino acid sequence of SEQ ID NO:170. In some embodiments, the CD3 variable heavy chain comprises a CDR1, CDR2, and CDR3 set forth in SEQ ID NO:186 and FIG. 2B. In some embodiments, the CD3 variable heavy chain comprises a CDR1, CDR2, and CDR3 as set forth in SEQ ID NOS:187-189, respectively. In some embodiments, the CD3 variable light chain comprises a CDR1, CDR2, and CDR3 set forth in SEQ ID NO:170 and FIG. 2A. In some embodiments, the CD3 variable heavy chain comprises a CDR1, CDR2, and CDR3 as set forth in SEQ ID NOS:171-173, respectively.

In some embodiments, the pseudo variable heavy domain (CD3 VHi or αCD3 VHi) comprises the amino acid sequence of SEQ ID NO:190 and the pseudo variable light domain (CD3 VLi) comprises the amino acid sequence of SEQ ID NO:174. In some embodiments, CD3 VHi comprises a CDR′. CDR2, and CDR3 set forth in SEQ ID NO:190 and FIG. 2B. In some embodiments, CD3 VHi comprises a CDR1, CDR2, and CDR3 set forth in SEQ ID NOS:191-193, respectively and FIG. 2B. In some embodiments, CD3 VLi comprises a CDR1, CDR2, and CDR3 set forth in SEQ ID NO:174 and FIG. 2A. In some embodiments, CD3 VLi comprises a CDR1, CDR2, and CDR3 set forth in SEQ ID NOS:175-177, respectively and FIG. 2A.

In some embodiments, the pseudo variable heavy domain (CD3 VHi2 or αCD3 VHi2) comprises the amino acid sequence of SEQ ID NO:194 and the pseudo variable light domain (CD3 VLi2 or αCD3 VLi2) comprises the amino acid sequence of SEQ ID NO:178. In some embodiments, CD3 VHi2 comprises a CDR1, CDR2, and CDR3 set forth in SEQ ID NO:194 and FIG. 2B. In some embodiments, CD3 VHi comprises a CDR1, CDR2, and CDR3 set forth in SEQ ID NOS: 195-197, respectively and FIG. 2B. In some embodiments, CD3 VLi2 comprises a CDR1, CDR2, and CDR3 set forth in SEQ ID NO:178 and FIG. 2A. In some embodiments, CD3 VLi2 comprises a CDR1, CDR2, and CDR3 set forth in SEQ ID NOS:179-181, respectively and FIG. 2A.

In some embodiments, the pseudo variable heavy domain (CD3 VHiGL4 or αCD3 VHiGL4) comprises the amino acid sequence of SEQ ID NO:198 and the pseudo variable light domain (CD3 VLiGL or αCD3 VLiGL) comprises the amino acid sequence of SEQ ID NO:182. In some embodiments, CD3 VHiGL4 comprises a CDR1, CDR2, and CDR3 set forth in SEQ ID NO:198 and FIG. 2B. In some embodiments, CD3 VHiGL4 comprises a CDR1, CDR2, and CDR3 set forth in SEQ ID NOS:199-201, respectively and FIG. 2B.

In some embodiments, CD3 VLiGL comprises a CDR1, CDR2, and CDR3 set forth in SEQ ID NO:182 and FIG. 2A. In some embodiments, CD3 VLiGL comprises a CDR1, CDR2, and CDR3 set forth in SEQ ID NOS:183-185, respectively and FIG. 2A.

In some embodiments, the TTA is selected from the group consisting of EGFR, FOLR1, B7H3, EpCAM, Trop2, and CA9.

In some embodiments, the first and second sdABDs bind to the same TTA and/or the third and fourth sdABDs bind to the same TTA. In some embodiments, the first, second, third, and fourth sdABDs bind to the same TTA. In some embodiments, the first and second sdABD-TTAs are the same and/or the third and fourth sdABD-TTAs are the same. In some embodiments, the first and second sdABD-TTAs are different and/or the third and fourth sdABD-TTAs are different. In some embodiments, the first, second, third, and fourth sdABDs bind to the different TTAs.

In some embodiments, the sdABD(s) is selected from the group consisting of SEQ ID NOS:50, 54, 58, 62, 66, 70, 74, 78, 82, 86, 90, 94, 98, 102, 16, 110, 114, 118, 122, 126, 130, 134, 138, 142, 146, 151, 154, 158, 162, and 166.

In some embodiments, the first and/or the second cleavable linker are cleaved by a human protease selected from the group consisting of MMP2, MMP9, meprin, Cathepsin S, Cathepsin K, Cathespin L, GranzymeB, uPA, Kallekriein7, matriptase and thrombin. In some embodiments, the first cleavable linker comprises an amino acid sequence depicted in FIGS. 3A-3D. In certain embodiments, the first cleavable linker comprises an amino acid sequence selected from the group consisting of SEQ ID NOS:210-281. In some embodiments, the second cleavable linker comprises an amino acid sequence depicted in FIGS. 3A-3D. In certain embodiments, the optional cleavable linker comprises an amino acid sequence selected from the group consisting of SEQ ID NOS:210-281.

In some embodiments, the first and/or the second optional cleavable linker are cleaved by a human protease selected from the group consisting of MMP2, MMP9, meprin, Cathepsin S, Cathepsin K, Cathespin L, GranzymeB, uPA, Kallekriein7, matriptase and thrombin. In some embodiments, the first optional cleavable linker comprises an amino acid sequence depicted in FIGS. 3A-3D. In certain embodiments, the first optional cleavable linker comprises an amino acid sequence selected from the group consisting of SEQ ID NOS:210-281. In some embodiments, the second optional cleavable linker comprises an amino acid sequence depicted in FIGS. 3A-3D. In certain embodiments, the second optional cleavable linker comprises an amino acid sequence selected from the group consisting of SEQ ID NOS:210-281.

In some embodiments, the first Fc monomer of the heterodimeric protein comprises an amino acid sequence selected from the group consisting of Pro584 (SEQ ID NO:44), Pro585 (SEQ ID NO:45), and Pro586 (SEQ ID NO:46). In some embodiments, second Fc monomer of the heterodimeric protein comprises an amino acid sequence of Pro575 (SEQ ID NO:42) or Pro576 (SEQ ID NO:43). In some embodiments, second Fc monomer of the heterodimeric protein comprises an amino acid sequence of Pro689 (SEQ ID NO:48).

In one aspect, provided herein is a heterodimeric protein composition comprising:
(a) a first Fc monomer comprising, from N- to C-terminal:
   i) a first single domain antigen binding domain (sdABD) that binds to a first tumor target antigen (TTA) (sdABD-TTA);
   ii) an optional domain linker;
   iii) a first constrained Fv domain comprising:
      1) a first variable heavy domain comprising vhCDR1, vhCDR2, and vhCDR3;
      2) a first constrained, non-cleavable linker (CNCL); and
      3) a first variable light domain comprising vlCDR1, vlCDR2, and vlCDR3;
   iv) a second sdABD-TTA;
   v) a first cleavable linker; and
   vi) a first Fc domain; and
(b) a second Fc monomer comprising, from N- to C terminal:
   i) a first pseudo Fv domain comprising:
      1) a first pseudo variable light domain;
      2) a non-cleavable linker; and
      3) a first pseudo variable heavy domain;
   ii) a second cleavable linker; and
   iii) a second Fc domain;
wherein said first Fc domain and said second Fc domain comprise a knob-in hole modification; wherein said variable heavy domain and said variable light domain are capable of binding human CD3 but said constrained Fv domains do not bind CD3; wherein said variable heavy domain and said pseudo variable light domain intermolecularly associate to form an inactive Fv; and wherein said variable light domain and said pseudo variable heavy domain intermolecularly associate to form an inactive Fv. In some embodiments, the first Fc domain comprises an Fc-knob modification and the second Fc domain comprises an Fc-hole modification. In some embodiments, the first Fc domain comprises an Fc-hole modification and the second Fc domain comprises an Fc-knob modification. In some embodiments, the Fc domain comprises a hinge, CH2, and CH3 domain. In some embodiments, the Fc domain comprises a CH2 and CH3 domain.

In some embodiments, the CD3 variable heavy chain (CD3 VH or αCD3 VH) comprises the amino acid sequence of SEQ ID NO:186 and the CD3 variable light domain (CD3 VL or αCD3 VL) comprises the amino acid sequence of SEQ ID NO:170. In some embodiments, the CD3 variable heavy chain comprises a CDR1, CDR2, and CDR3 set forth in SEQ ID NO:186 and FIG. 2B. In some embodiments, the CD3 variable heavy chain comprises a CDR1, CDR2, and CDR3 as set forth in SEQ ID NOS:187-189, respectively. In some embodiments, the CD3 variable light chain comprises a CDR1, CDR2, and CDR3 set forth in SEQ ID NO:170 and FIG. 2A. In some embodiments, the CD3 variable heavy chain comprises a CDR1, CDR2, and CDR3 as set forth in SEQ ID NOS:171-173, respectively.

In some embodiments, the pseudo variable heavy domain (CD3 VHi or αCD3 VHi) comprises the amino acid sequence of SEQ ID NO:190 and the pseudo variable light domain (CD3 VLi) comprises the amino acid sequence of SEQ ID NO:174. In some embodiments, CD3 VHi comprises a CDR1, CDR2, and CDR3 set forth in SEQ ID NO:190 and FIG. 2B. In some embodiments, CD3 VHi comprises a CDR1, CDR2, and CDR3 set forth in SEQ ID NOS:191-193, respectively and FIG. 2B. In some embodiments, CD3 VLi comprises a CDR1, CDR2, and CDR3 set forth in SEQ ID NO:174 and FIG. 2A. In some embodiments, CD3 VLi comprises a CDR1, CDR2, and CDR3 set forth in SEQ ID NOS:175-177, respectively and FIG. 2A.

In some embodiments, the pseudo variable heavy domain (CD3 VHi2 or αCD3 VHi2) comprises the amino acid sequence of SEQ ID NO:194 and the pseudo variable light domain (CD3 VLi2 or αCD3 VLi2) comprises the amino acid sequence of SEQ ID NO:178. In some embodiments, CD3 VHi2 comprises a CDR1, CDR2, and CDR3 set forth in SEQ ID NO:194 and FIG. 2B. In some embodiments, CD3 VHi comprises a CDR1, CDR2, and CDR3 set forth in SEQ ID NOS: 195-197, respectively and FIG. 2B. In some embodiments, CD3 VLi2 comprises a CDR1, CDR2, and CDR3 set forth in SEQ ID NO:178 and FIG. 2A. In some embodiments, CD3 VLi2 comprises a CDR1, CDR2, and CDR3 set forth in SEQ ID NOS:179-181, respectively and FIG. 2A.

In some embodiments, the pseudo variable heavy domain (CD3 VHiGL4 or αCD3 VHiGL4) comprises the amino acid sequence of SEQ ID NO:198 and the pseudo variable light domain (CD3 VLiGL or αCD3 VLiGL) comprises the amino acid sequence of SEQ ID NO:182. In some embodiments, CD3 VHiGL4 comprises a CDR1, CDR2, and CDR3 set forth in SEQ ID NO:198 and FIG. 2B. In some embodiments, CD3 VHiGL4 comprises a CDR1, CDR2, and CDR3 set forth in SEQ ID NOS:199-201, respectively and FIG. 2B.

In some embodiments, CD3 VLiGL comprises a CDR1, CDR2, and CDR3 set forth in SEQ ID NO:182 and FIG. 2A. In some embodiments, CD3 VLiGL comprises a CDR1, CDR2, and CDR3 set forth in SEQ ID NOS:183-185, respectively and FIG. 2A.

In some embodiments, the TTA is selected from the group consisting of EGFR, FOLR1, B7H3, EpCAM, Trop2, and CA9.

In some embodiments, the first and second sdABDs bind to the same TTA. In some embodiments, the first and second sdABDs bind to different TTAs. In some embodiments, the first and second sdABD-TTAs are the same. In some embodiments, the first and second sdABD-TTAs are different.

In some embodiments, the sdABD(s) is selected from the group consisting of SEQ ID NOS:50, 54, 58, 62, 66, 70, 74, 78, 82, 86, 90, 94, 98, 102, 16, 110, 114, 118, 122, 126, 130, 134, 138, 142, 146, 151, 154, 158, 162, and 166. In some embodiments, an sdABD that binds to a specific TTA is selected from the group consisting of SEQ ID NOS:50, 54, 58, 62, 66, 70, 74, 78, 82, 86, 90, 94, 98, 102, 16, 110, 114, 118, 122, 126, 130, 134, 138, 142, 146, 151, 154, 158, 162, and 166. In some embodiments, the sdABD comprises a CDR1, CDR2, and CDR3 as set forth in SEQ ID NOS:50, 54, 58, 62, 66, 70, 74, 78, 82, 86, 90, 94, 98, 102, 16, 110, 114, 118, 122, 126, 130, 134, 138, 142, 146, 151, 154, 158, 162, and 166. In some embodiments, the sdABD comprises a CDR1, CDR2, and CDR3 as set forth in FIG. 1.

In some embodiments, the first cleavable linker and/or the second cleavable linker are cleaved by a human protease selected from the group consisting of MMP2, MMP9, meprin, Cathepsin S, Cathepsin K, Cathespin L, GranzymeB, uPA, Kallekriein7, matriptase and thrombin. In some embodiments, the cleavable linker comprises an amino acid sequence depicted in FIGS. 3A-3D. In certain embodiments, the first cleavable linker comprises an amino acid sequence selected from the group consisting of SEQ ID NOS:210-281. In some embodiments, the second cleavable linker comprises an amino acid sequence depicted in FIGS. 3A-3D. In certain embodiments, the second cleavable linker comprises an amino acid sequence selected from the group consisting of SEQ ID NOS:210-281.

Also, provided herein are nucleic acid compositions, expression vectors, host cells, and method for making the homodimeric and heterodimer proteins described herein.

Also, provided is a method for treating cancer in a subject, e.g., a human subject comprising administering any one of the homodimeric proteins or heterodimeric proteins described herein.

This application makes reference to International Published Patent Application No. WO2017/156178, filed Mar. 8, 2017, U.S. Provisional Application No. 62/305,092, filed Mar. 8, 2016, U.S. Provisional Application No. 62/555,943, filed Sep. 8, 2017. U.S. Provisional Application No. 62/555,999, filed Sep. 8, 2017, U.S. Provisional Application No. 62/583,327, filed Nov. 15, 2017, and U.S. Provisional Application No. 62/587,318, filed Nov. 16, 2017, U.S. Provisional Application No. 62/555,999, filed on Sep. 8, 2017, U.S. Provisional Application No. 62/555,943 filed Sep. 8, 2017, and International Patent Application No. PCT/US2018/049798 filed Sep. 6, 2018, the disclosures in their entirety are herein incorporated by reference, including the figures, figure legends, and definitions, as well as all recited embodiments.

### BRIEF DESCRIPTION OF THE DRAWINGS

FIG. 1A-FIG. 1G depict a number of sdABD-TTA sequences of the invention. For antigen binding domains, the CDRs are bold, underlined.

FIG. 2A-FIG. 2C depict a number of anti-CD3 scFv domain and anti-HSA sequences of the invention. For antigen binding domains, the CDRs are bold, underlined.

FIG. 3A-FIG. 3F depicts a number of suitable protease cleavage sites and non-cleavable or domain linkers. As will be appreciated by those in the art, these cleavage sites can be used as cleavable linkers. In some embodiments, for example when more flexible cleavable linkers are required, there can be additional amino acids (generally glycines and serines) that are either of both N- and C-terminal to these cleavage sites. In some instances, the “/” marks the cleavage site of the linker.

FIG. 4 shows an exemplary embodiment of a conditionally activated binding polypeptide of the “construct 1” format comprising Fc hole/knob regions. A schematic of a Pro37+Pro36 prodrug construct is shown and the resulting bispecific polypeptide after enterokinase (EK) cleavage, although other cleavage sites such as described herein can be used. The bispecific polypeptide includes sdABDs that bind EGFR and a Fv domain that binds CD3. It should be noted that sdABDs that bind other target tumor antigens (TTA) such as, but not limited to, FOLR1, B7H3, EpCAM, EGFR, Trop2, and CA9 can be used in other embodiments. In addition, FIG. 4 shows the use of two different protein “tags” at the C-terminus of the Fc domains, that were used to facilitate purification of the heterodimeric proteins of the invention, but as will be appreciated by those in the art, these can be removed.

FIG. 5 shows an exemplary embodiment of a conditionally activated binding polypeptide of the “construct 2” format comprising Fc hole/knob regions. A schematic of a Pro38+Pro36 prodrug construct is shown, again using the Flag cleavage site for EK, although many embodiments utilize other cleavage sites. It should be noted that sdABDs that bind other target tumor antigens (TTA) such as, but not limited to, FOLR1, B7H3, EpCAM, EGFR, Trop2, and CA9 can be used in other embodiments. In addition, FIG. 5 shows the use of two different protein “tags” at the C-terminus of the Fc domains, that were used to facilitate purification of the heterodimeric proteins of the invention, but as will be appreciated by those in the art, these can be removed.

FIG. 6A-FIG. 6B show that some illustrative heterodimeric Fc prodrug constructs described herein displayed low or a lack of conditionality upon cleavage with a cognate protease in a TDCC assay. In FIG. 6A, Pro36+37 (circles) was not pretreated with EK protease, while Pro36+37 cleaved (squares) was. In FIG. 6B, Pro36+38 (circles) was not pretreated with EK protease, while Pro36+38 cleaved (squares) was. Pro214 is a full-length negative control (open squares) and Pro 51 (triangle) is a positive control that does not require protease cleavage for activity.

FIG. 7 shows an exemplary embodiment of a conditionally activated binding polypeptide of the “construct 3” format comprising Fc hole/knob regions. A schematic of a Pro68+Pro67 prodrug construct is shown and the resulting bispecific polypeptide after enterokinase (EK) cleavage, again using the Flag cleavage site for EK, although many embodiments utilize other cleavage sites. The bispecific polypeptide includes sdABDs that bind EGFR and a Fv domain that binds CD3. It should be noted that sdABDs that bind other target tumor antigens (TTA) such as, but not limited to, FOLR1, B7H3, EpCAM, EGFR, Trop2, and CA9 can be used in other embodiments. In addition, FIG. 7 shows the use of two different protein “tags” at the C-terminus of the Fc domains, that were used to facilitate purification of the heterodimeric proteins of the invention, but as will be appreciated by those in the art, these can be removed.

FIG. 8 shows an exemplary embodiment of a conditionally activated binding polypeptide of the “construct 4” format comprising Fc hole/knob regions. A schematic of a Pro69+Pro70 prodrug construct is shown, again using the Flag cleavage site for EK, although many embodiments utilize other cleavage sites. It should be noted that sdABDs that bind other target tumor antigens (TTA) such as, but not limited to, FOLR1, B7H3, EpCAM, EGFR, Trop2, and CA9 can be used in other embodiments. In addition, FIG. 8 shows the use of two different protein “tags” at the C-terminus of the monomer proteins, that were used to facilitate purification of the heterodimeric proteins of the invention, but as will be appreciated by those in the art, these can be removed.

FIG. 9 shows an exemplary embodiment of a conditionally activated binding polypeptide of the “construct 5” format comprising Fc hole/knob regions. A schematic of a Pro71+Pro67 prodrug construct is shown, again using the Flag cleavage site for EK, although many embodiments utilize other cleavage sites. It should be noted that sdABDs that bind other target tumor antigens (TTA) such as, but not limited to, FOLR1, B7H3, EpCAM, EGFR, Trop2, and CA9 can be used in other embodiments. In addition, FIG. 9 shows the use of two different protein “tags” at the C-terminus of the monomer proteins, that were used to facilitate purification of the heterodimeric proteins of the invention, but as will be appreciated by those in the art, these can be removed.

FIG. 10A-FIG. 10C show that some illustrative heterodimeric Fc prodrug constructs described herein displayed conditionality but lacked high activity when cleaved with a cognate protease in a TDCC assay. In FIG. 10A, Pro67+68 (circles) was not pretreated with EK protease, while Pro67+68 cleaved (squares) was. In FIG. 10B, Pro69+70 (circles) was not pretreated with EK protease, while Pro69+70 cleaved (squares) was. In FIG. 10C, Pro67+71 (circles) was not pretreated with EK protease, while Pro67+71 cleaved (squares) was. Pro214 is a full-length negative control (open squares) and Pro 51 (triangle) is a positive control that does not require protease cleavage for activity.

FIG. 11 shows an exemplary embodiment of a conditionally activated binding polypeptide of the “construct 6” format comprising Fc hole/knob regions. A schematic of a Pro219+Pro218 prodrug construct is shown, using an MMP9 protease cleavage site, although others as described herein can be used as well. It should be noted that sdABDs that bind other target tumor antigens (TTA) such as, but not limited to, FOLR1, B7H3, EpCAM, EGFR, Trop2, and CA9 can be used in other embodiments. In addition, FIG. 11 shows the use of two different protein “tags” at the C-terminus of the monomer proteins, that were used to facilitate purification of the heterodimeric proteins of the invention, but as will be appreciated by those in the art, these can be removed.

FIG. 12 shows an exemplary embodiment of a conditionally activated binding polypeptide of the “construct 7” format comprising Fc hole/knob regions. A schematic of a Pro217+Pro218 prodrug construct is shown, using an MMP9 protease cleavage site, although others as described herein can be used as well. It should be noted that sdABDs that bind other target tumor antigens (TTA) such as, but not limited to, FOLR1, B7H3, EpCAM, EGFR, Trop2, and CA9 can be used in other embodiments. In addition, FIG. 12 shows the use of two different protein “tags” at the C-terminus of the monomer proteins, that were used to facilitate purification of the heterodimeric proteins of the invention, but as will be appreciated by those in the art, these can be removed.

FIG. 13A-FIG. 13B show that illustrative heterodimeric Fc prodrug constructs described herein displayed conditionality and high potency when cleaved with a cognate protease in a TDCC assay. In FIG. 13A, Pro217+218 (circles) was not pretreated with EK protease, while Pro217+218 cleaved (squares) was. In FIG. 13B, Pro218+219 (circles) was not pretreated with EK protease, while Pro218+219 cleaved (squares) was. Pro214 is a full-length negative control (open squares) and Pro 51 (triangle) is a positive control that does not require protease cleavage for activity.

FIG. 14A-FIG. 14G depict exemplary sequences of the invention. Linkers are underlined, with cleavable linkers single/double underlined and italicized. CDRs are bold, underlined. Slashes (“/”) depict domain separators. C-terminal tags such as maltose-binding protein (MBP), (His)10 and Strep-Tag® II tags are bold, but as outlined herein, are optional, depending on the purification scheme used. Thus, included within the description herein are the sequences of FIG. 14 that exclude the C-terminal tags.

FIG. 15A-FIG. 15C depict additional Pro219 constructs (FIG. 15A) and additional Pro217 constructs (FIG. 15B and FIG. 15C).

FIG. 16A-FIG. 16G depict additional sequences of the invention. For antigen binding domains, the CDRs are bold, underlined. “/”s indicate the intersection of domains, domain linkers are underlined, and cleavable linkers are single/double-underlined and italicized. Many of the constructs include histidine tags, which are optional, depending on the use.

FIG. 17A-FIG. 17C depict additional sequences of the invention such as Pro556, Pro557, Pro587, Pro588, and Pro589. For antigen binding domains, the CDRs are bold, underlined. “/”s indicate the intersection of domains, domain linkers are underlined, and cleavable linkers are single/double underlined and italicized. Many of the constructs include histidine tags, which are optional, depending on the use. In exemplary embodiments, the sequences provide are used to make a homodimeric Fc fusion prodrug protein such that the Fc domains form a homodimer.

FIG. 18A-FIG. 18C depict additional sequences of the invention such as Pro574, Pro575, Pro576, Pro584, Pro585, and Pro586. For antigen binding domains, the CDRs are bold, underlined. “/”s indicate the intersection of domains, domain linkers are underlined, and cleavable linkers are single/double-underlined and italicized. Many of the constructs include histidine tags, which are optional, depending on the use. In exemplary embodiments, the sequences provide are used to make an Fc fusion heterodimer. In some embodiments, the heterodimeric Fc fusion prodrug protein comprises a pair selected from the group consisting of Pro575 and Pro574, Pro575 and Pro584, Pro575 and Pro585, Pro575 and Pro586, Pro576 and Pro574, Pro576 and Pro584, Pro576 and Pro585, and Pro576 and Pro586.

FIG. 19 shows that some illustrative homodimeric Fc prodrug constructs described herein displayed conditionality and activity when cleaved with a cognate protease in a TDCC assay. In FIG. 19, Pro556 (circles) was not pretreated with protease, while Pro556 cleaved (squares) was. Pro557 contains a noncleavable linker N-terminal to the pseudo Fv domain (black triangles). Prol 86 (diamonds) is not a homodimeric Fc prodrug construct. Pro186 from N- to C-terminal comprises a) an sdABD-EGFR, b) a constrained Fv domain, c) an sdABD-EGFR, d) a cleavable linker, e) a pseudo Fv domain, and f) an sdABD-HSA.

FIG. 20A-FIG. 20B depict additional sequences of the invention such as Pro688, Pro689, and Pro690. For antigen binding domains, the CDRs are underlined. “/”s indicate the intersection of domains, domain linkers are double-underlined, and cleavable linkers are double-underlined and italicized. Many of the constructs include histidine tags, which are optional, depending on the use. In exemplary embodiments, the sequences provide are used to make an Fc fusion heterodimer.

FIG. 21 depicts exemplary schemes of the TTA-binding heterodimeric Fc proteins of the invention such as Pro574, Pro575, and Pro577.

FIG. 22 depicts the potency of EGFR-binding heterodimeric Fc proteins of the invention such as Pro574, Pro575, and Pro577.

FIG. 23 depicts the anti-tumor response of EGFR-binding heterodimeric Fc proteins of the invention such as Pro574, Pro575, and Pro577 in an adoptive T cell transfer mouse model.

### DETAILED DESCRIPTION OF THE INVENTION

### I. Introduction

The present invention is directed to methods of reducing the toxicity and side effects of bispecific antibodies (including antibody-like functional proteins) that bind to important physiological targets such as CD3 and tumor antigens. Many antigen binding proteins, such as antibodies, can have significant “on target/off-tumor” side effects, and thus there is a need to only activate the binding capabilities of a therapeutic molecule in the vicinity of the disease tissue, to avoid off-target interactions. Accordingly, the present invention is directed to multivalent conditionally effective (“MCE”) proteins that have a number of functional protein domains. In general, one of these domains is an antigen binding domain (ABD) that will bind a target tumor antigen (TTA). The another domain is an ABD that will bind a T-cell antigen such as CD3 under certain conditions, such as when a portion of the ABD is in close proximity to a complementary portion of the ABD to form an anti-CD3 Fv binding domain. That is, the therapeutic molecules are made in a “pro-drug” like format, wherein the CD3 binding domain is inactive until exposed to a tumor environment. To accomplish this conditionality, the invention utilizes “pseudo” or “inactive” or “inert” variable domains in several different ways, depending on the format, as is described herein and shown in the figures. These are referred to herein as “iVH” and “iVL” domains,

In some embodiments of the present invention, the CD3 binding domain (“CD3 Fv”) is in a constrained format, wherein the linker between the variable heavy and variable light domains that traditionally form an Fv is too short to allow the two domains to bind each other. In some embodiments, in the prodrug (e.g., uncleaved) format, the prodrug polypeptide also comprises a “pseudo Fv domain”. The pseudo Fv domain can comprise either a variable heavy domain with standard framework regions but “inert” or “dummy” CDRs (inactive variable heavy domain), a variable light domain with standard framework regions but “inert” or “dummy” CDRs (inactive variable light domain), or both. Thus, the constrained Fv domain will bind to the pseudo Fv domain, due to the affinity of the framework regions of each. However, due to the “inert” CDRs of the pseudo domain, the resulting ABDs will not bind CD3, thus preventing off target toxicities. However, in the presence of proteases that are in or near the tumor, the prodrug construct is cleaved in such a way as to allow the “real” variable heavy and variable light domains to associate, thus triggering active CD3 binding and the resulting tumor efficacy.

In other embodiments, in the prodrug format, the prodrug polypeptide comprises two pseudo Fv domains and an Fc domain linked to each pseudo Fv domain. The first pseudo Fv domain can comprise an inactive variable heavy domain and an active variable light domain and the second pseudo Fv domain can comprise an active variable heavy domain and an inactive variable light domain. The ABDs in the prodrug format will not bind CD3 in proteolytically inactive tissues. Yet, in or near the tumor, proteases can cleave the prodrug constructs such that the active variable heavy and active variable light domains can associate and bind CD3, thereby inducing target tumor cell cytoxicity.

Thus, the prodrug constructs provided herein comprise a heterodimeric IgG Fc region that form a “knobs-into-holes” (“KIH”) conformation. Detailed descriptions of the knobs-into-holes concept can be found, for example, in U.S. Pat. Nos. 5,731,168 and 7,186,076; and Ridgway et al., Protein Engineering, Design and Selection, 1996, 9(7):617-621, Atwell et al., J Mol Biol, 1997, 270(1):26-35; Merchant et al., Nat Biotechnol, 1998, 16:677-681; and Carter, J. Immunological Methods, 2001, 24(1-2):7-15. Briefly, a knob can be created at the CH3 domain interface of the first IgG Pc chain by replacing a smaller amino acid side chain with a larger one (e.g., T366W); and a, hole can be created in the juxtaposed position at the CH3 interface of the second IgG Fe chain by replacing a larger amino acid side chain with a smaller one (e.g., Y407V). Suitable KIH variants are described below. Thus herein a C113 domain that has “knob substitution(s)” is referred to as “CH3-knob” and a CH3 domain with “hole substitution(s)” is referred to herein as “CH3-hole” with the generic term being “CH3-Kill” to cover both, since; as will be appreciated by those in the art, which “side” of the Fc dimer contains “hole variants” and which contains “knob variants” is not determinative and can vary.

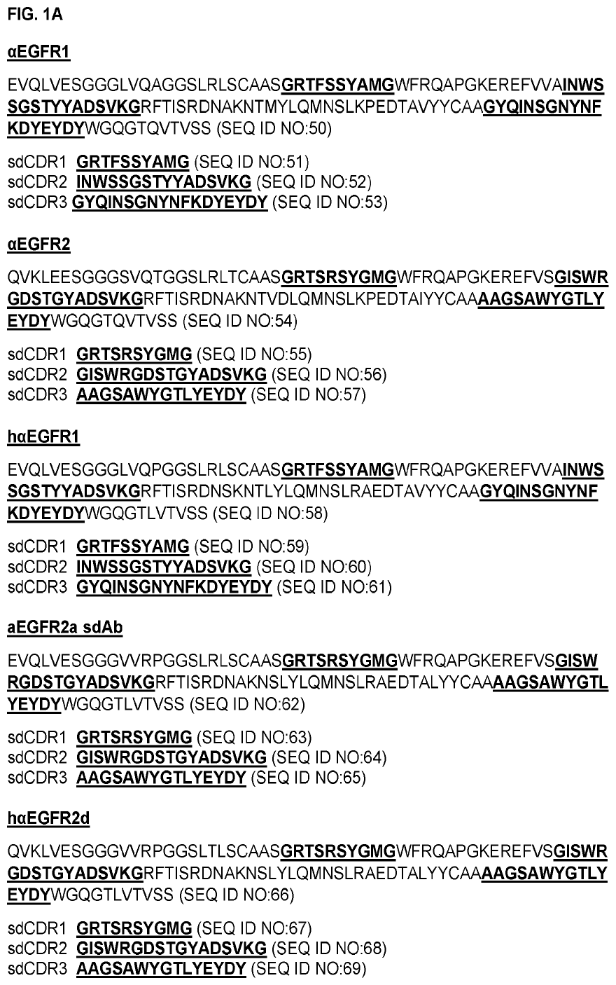

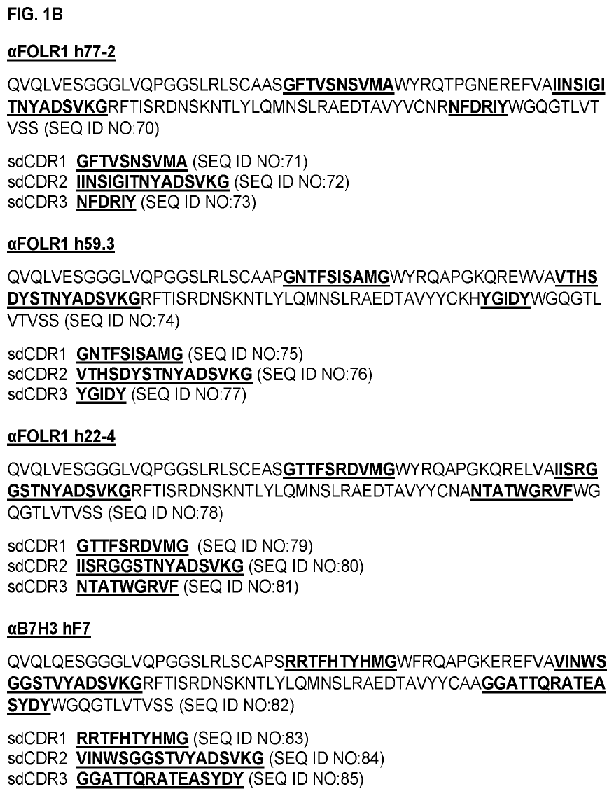

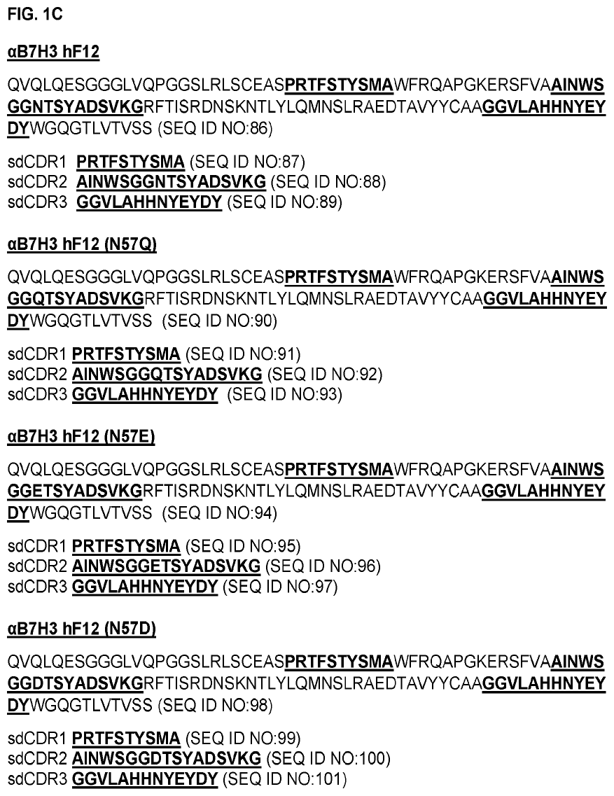

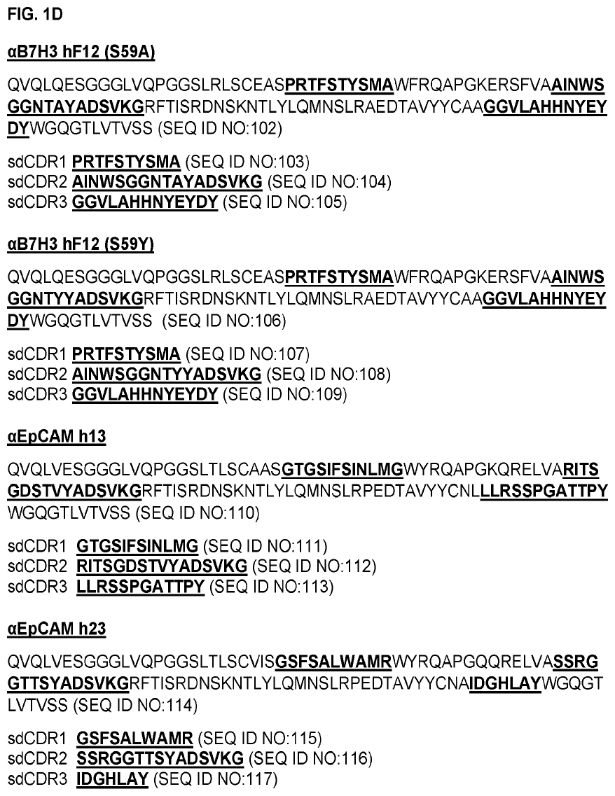

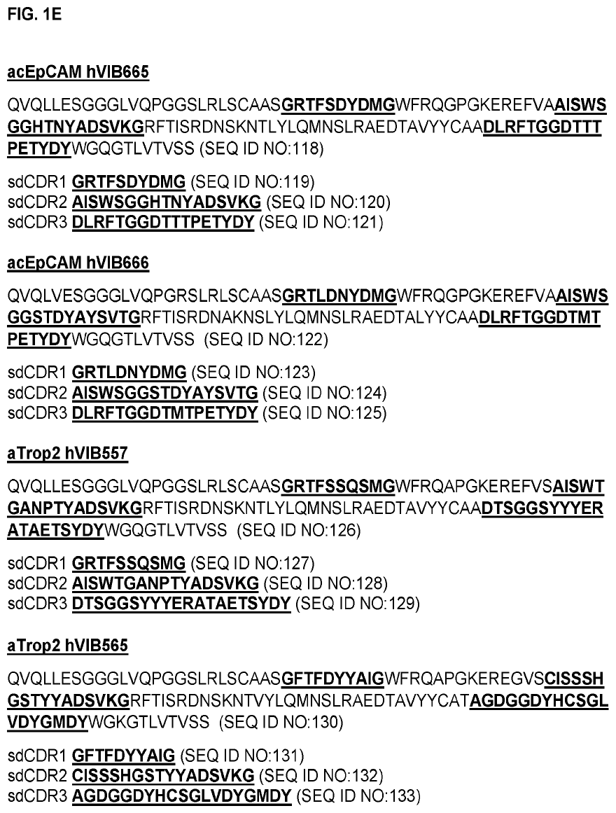

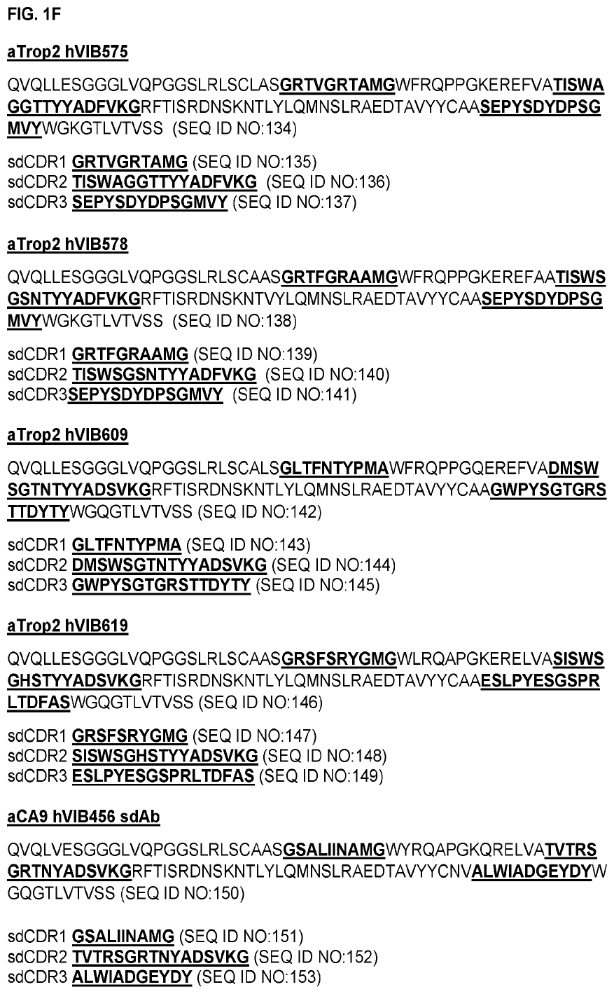

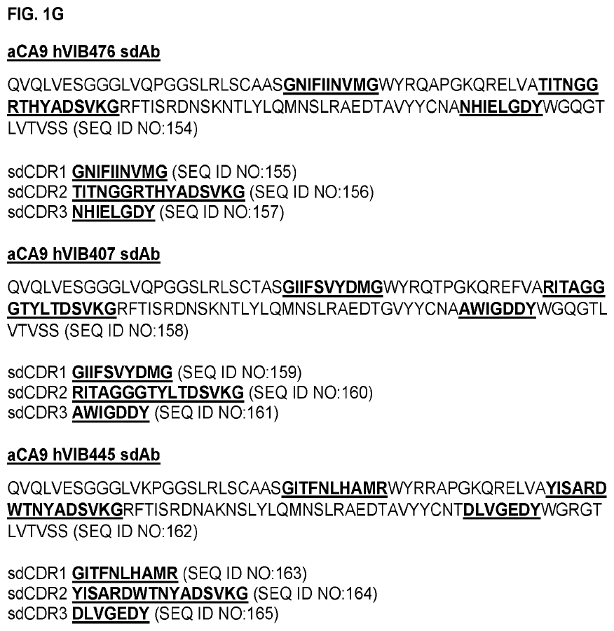

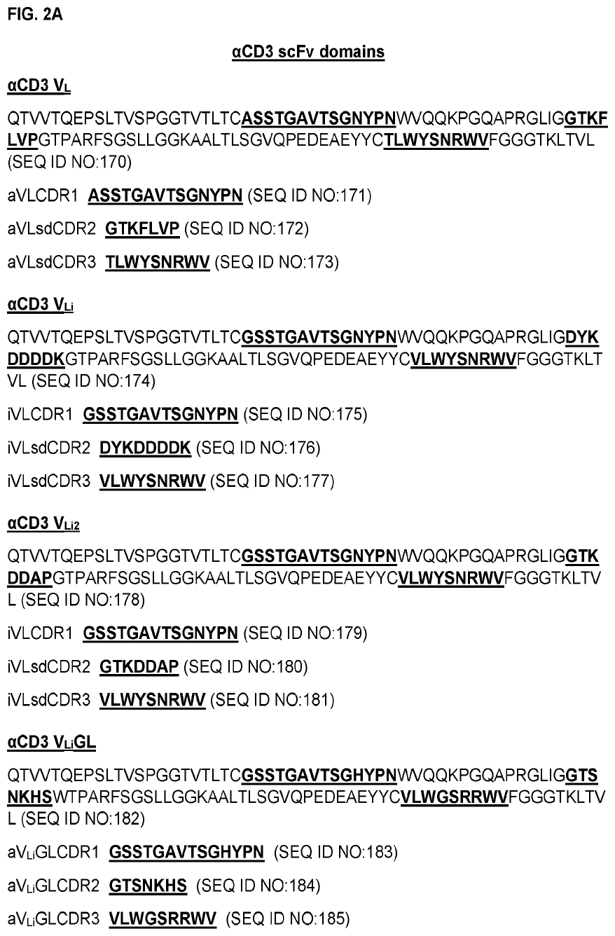

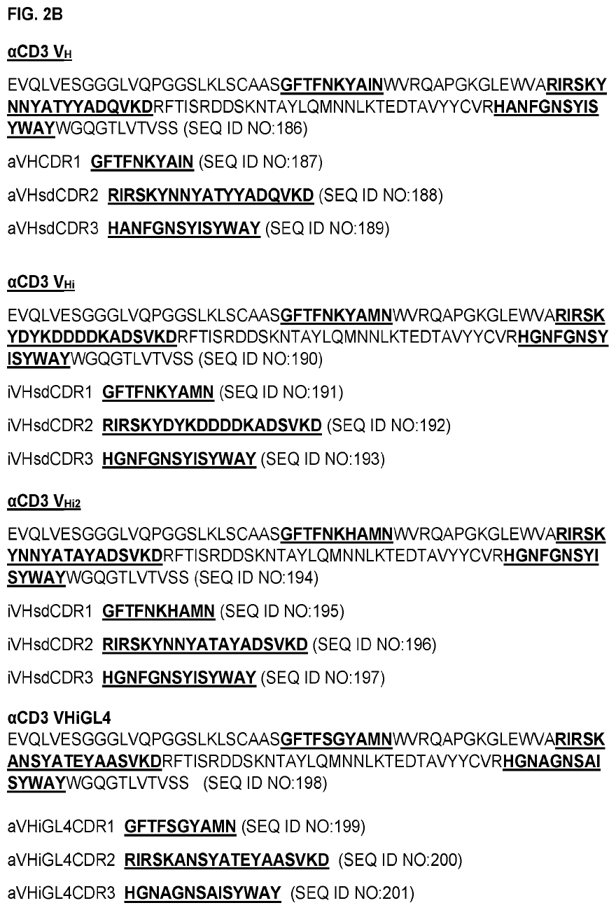

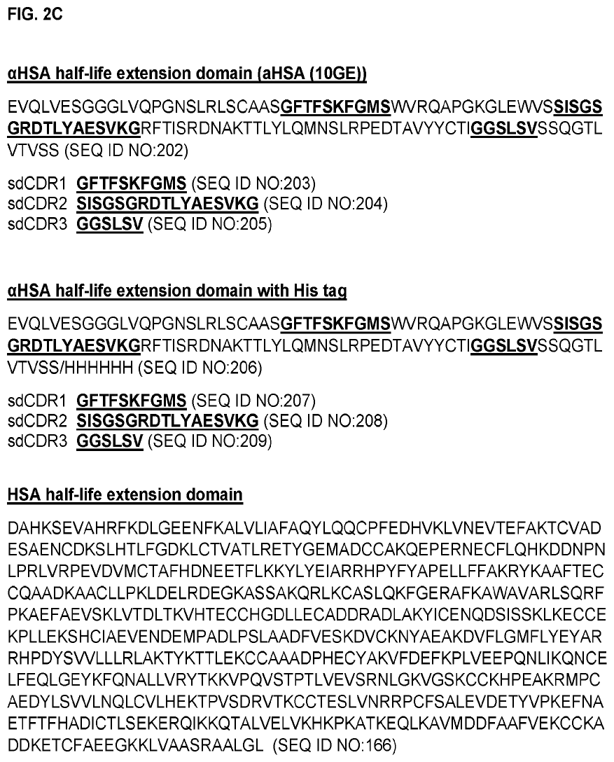

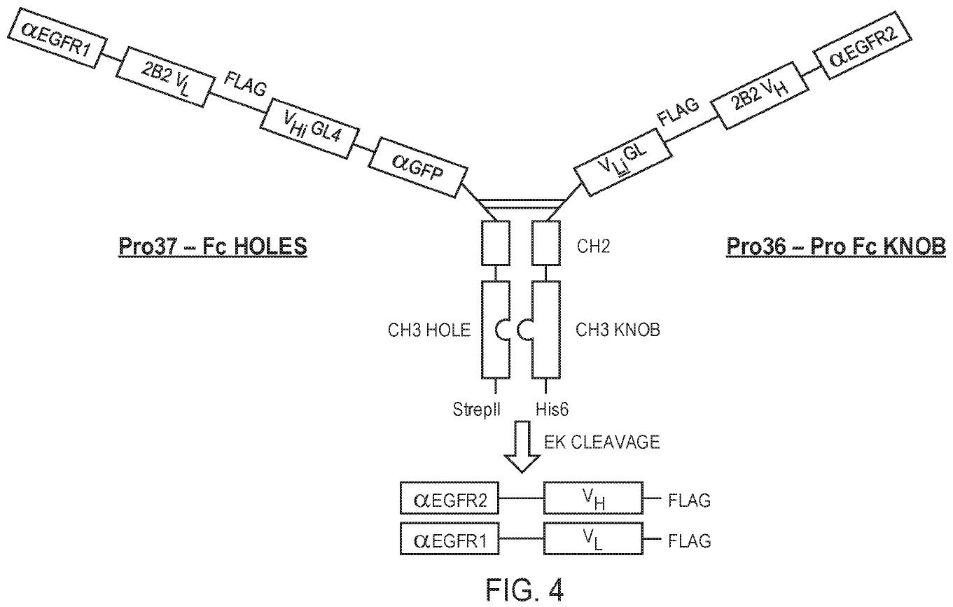

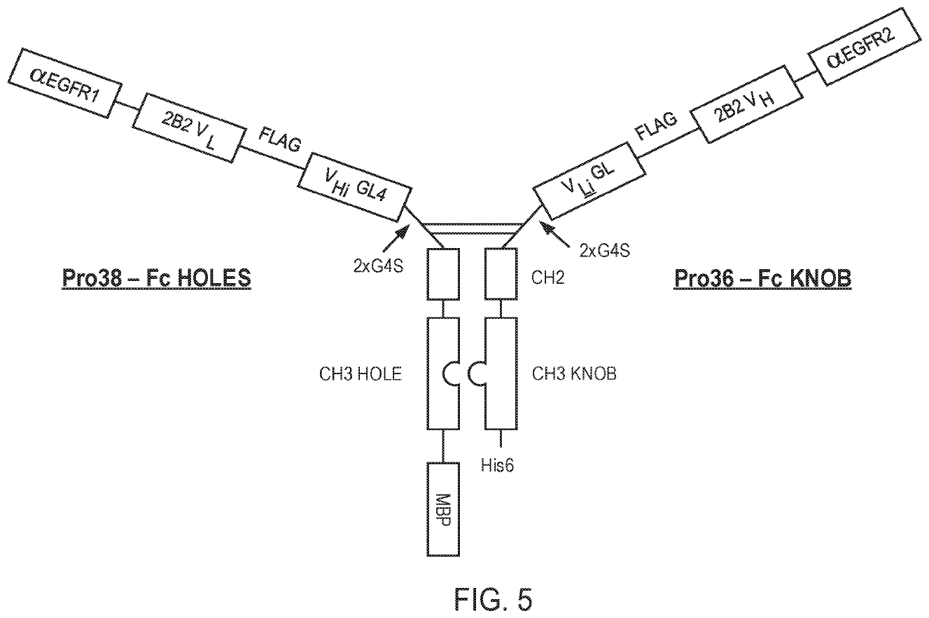

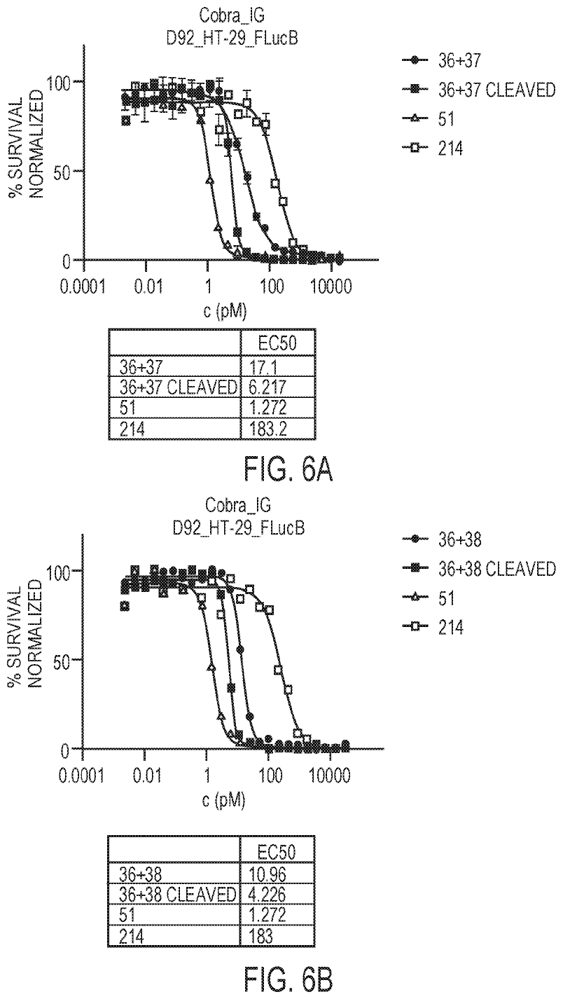

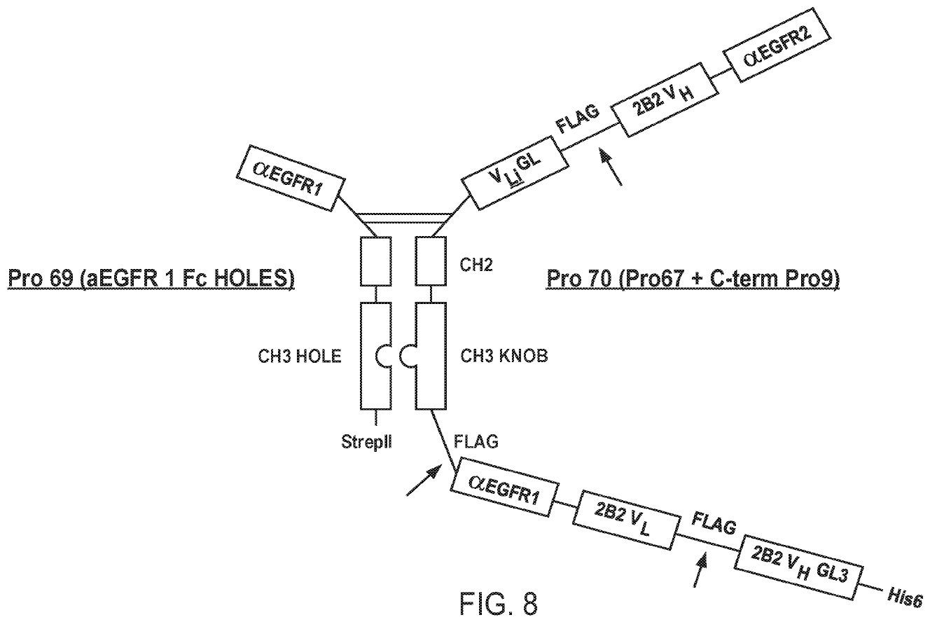

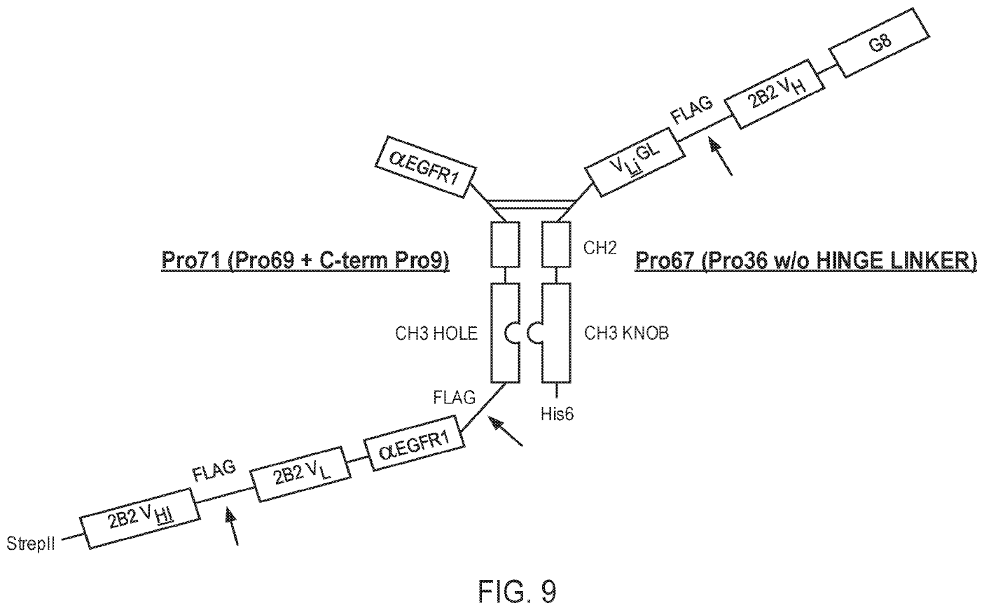

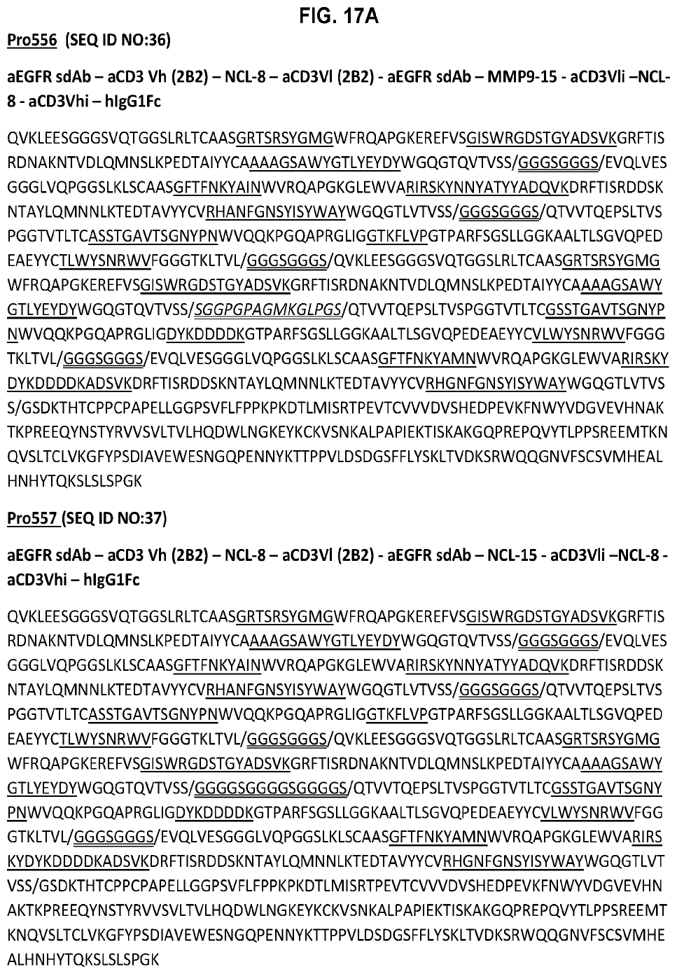

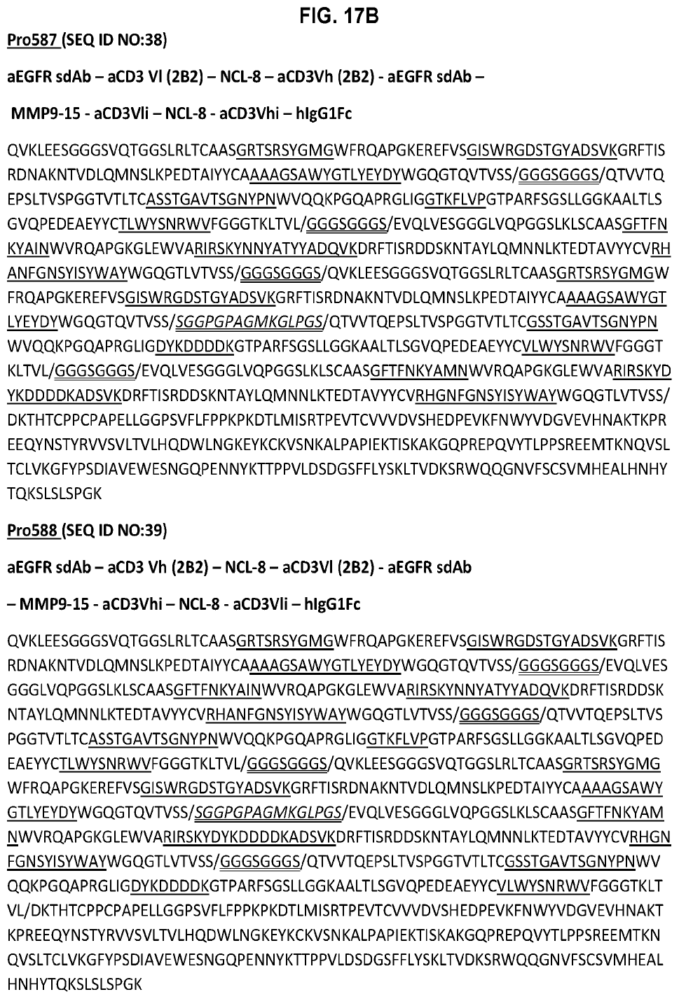

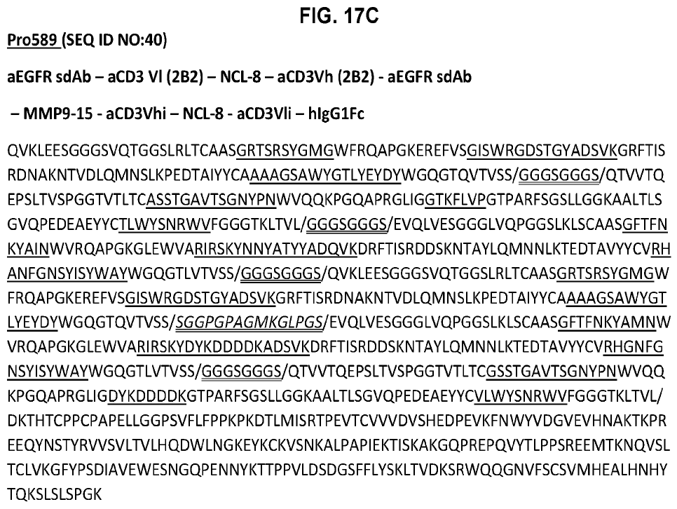

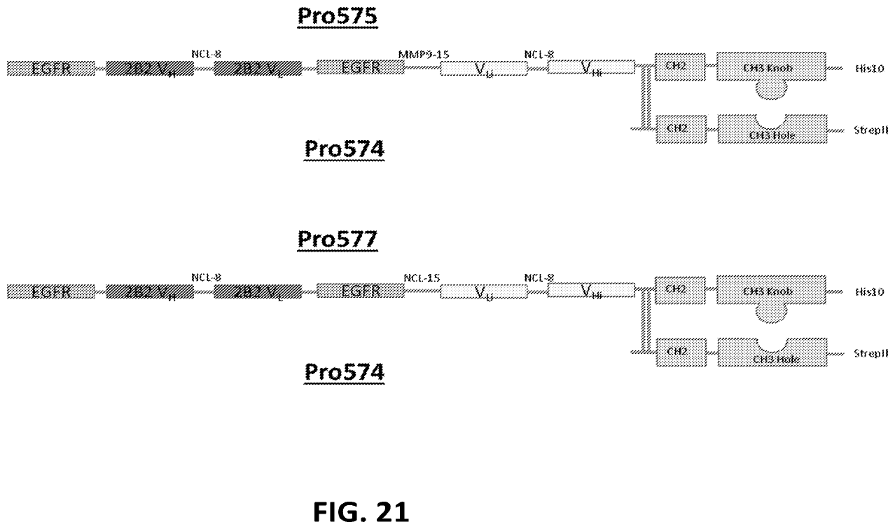

As discussed herein, there are a variety of conformations and formats that find use in the present invention. The conformation of the prodrug constructs can take on a wide variety of configurations, such that the prodrug activation can happen in several general ways, as in formats shown in FIG. 21 and sequence provided in FIGS. 17A-17C. Additional useful formats are shown in FIG. 1 as a “construct 1”, FIG. 2 as a “construct 2”, FIG. 4 as a “construct 3”, FIG. 5 as a “construct 4”, FIG. 6 as a “construct 5”, FIG. 8 as a “construct 6”, and FIG. 9 as a “construct 7” of WO2019/051122. These constructs rely generally on Fc domains that form heterodimeric Fc structures, to allow for the proper pre-cleavage association of inert binding domains.

In the “construct 1” embodiments, the prodrug construct includes a first Fc polypeptide comprising a CH2-CH3-hole polypeptide, a first pseudo Fv domain, and an antigen binding domain (ABD) that can bind a target tumor antigen (TTA); and a second Fc polypeptide comprising a CH2-CH3-knob polypeptide, a second pseudo Fv domain, and an antigen binding domain (ABD) that can bind a target tumor antigen (TTA). A pseudo Fv domains refer to CD3 binding domains (Fvs) that are inactive until exposed to a tumor environment. In this embodiment, a pseudo Fv domain comprises an active variable heavy domain (active VH) and an inactive variable light domain (inactive VL). In other embodiments, a pseudo Fv domain comprises an active variable light domain (active VL) and an inactive variable heavy domain (inactive VH). Additionally, as will be appreciated by those in the art, the pseudo Fv domains of “construct 1” can be, from N- to C-terminally, in either orientation, either VH-linker-VL or VL-linker-VH (for example, in FIG. 1, “construct 1”, the pseudo Fv domains are shown as VH-linker-VL, but this can be switched).

In some embodiments of “construct 1”, the first Fc polypeptide (from N-terminal to C-terminal) includes: an antigen binding domain for a first TTA linked via a domain linker to an active VL domain that is attached via a cleavable linker to an inactive VH domain that is linked to a second antigen binding domain that is linked to a CH2-CH3-KIH polypeptide; and the second Fc polypeptide (from N-terminal to C-terminal) includes: an antigen binding domain for a second TTA linked via a domain linker to an active VH domain that is attached via a cleavable linker to an inactive VL domain that is linked to a CH2-CH3-KIH polypeptide. In some cases the first Fc polypeptide contains the CH3-hole and the second contains the CH3-knob. Upon cleavage of the cleavable linker of the first Fc polypeptide and cleavage of the cleavable linker of the second Fc polypeptide at or near a tumor site, the active VL of the first Fc polypeptide and active VH of the second Fc polypeptide can associate and trigger active CD3 binding. In addition to the innate self-assembly of the active VH and VL domains, each domain is linked to an antigen binding domain to a tumor antigen. As such, the protease cleaved product can bind to tumor cells and recruit T cells to the tumor site. In some embodiments, the first TTA and the second TTA are the same tumor antigen. In other embodiments, the first TTA and the second TTA are different tumor antigens.

In some instances, the prodrug construct of “construct 1” has two cleavage sites: one between the active variable light chain and the inactive variable heavy chain of the first Fc polypeptide, and a second between the active variable heavy chain and the inactive variable light chain of the second Fc polypeptide. In some embodiments, the two cleavage sites are recognized and cleaved by the same protease. As such, the two cleavage sites can have the same or substantially the same amino acid sequence. In other embodiments, the two cleavage sites are recognized and cleaved by different protease. Thus, the two cleavage sites can have different amino acid sequences.

In some embodiments of “construct 2”, the prodrug construct includes a first Fc polypeptide comprising a CH2-CH3-KIH polypeptide, a first pseudo Fv domain comprising an active variable light chain (active VL) and an inactive variable heavy chain (inactive VH), and an antigen binding domain (ABD) that can bind a target tumor antigen (TTA); and a second Fc polypeptide comprising a CH2-CH3-KIH polypeptide, a second pseudo Fv domain comprising an active variable heavy chain (active VH) and an inactive variable light chain (inactive VL), and an antigen binding domain (ABD) that can bind a target tumor antigen (TTA). In some cases, the first Fc polypeptide contains the CH3-hole and the second contains the CH3-knob.

In some embodiments of “construct 2”, the first Fc polypeptide (from N-terminal to C-terminal) includes: an antigen binding domain for a first TTA linked via a domain linker to an active VL domain that is attached via a cleavable linker to an inactive VH domain that is linked via a domain linker to a CH2-CH3-KIH polypeptide; and the second Fc polypeptide (from N-terminal to C-terminal) includes: an antigen binding domain for a second TTA linked via a domain linker to an active VH domain that is attached via a cleavable linker to an inactive VL domain that is linked via an domain linker to a CH2-CH3-KIH polypeptide. In some cases, the first Fc polypeptide contains the CH3-hole and the second contains the CH3-knob. Upon cleavage of the cleavable linker of the first Fc polypeptide and cleavage of the cleavable linker of the second Fc polypeptide at or near a tumor site, the active VL of the first Fc polypeptide and active VH of the second Fc polypeptide can associate and trigger active CD3 binding. In addition to the innate self-assembly of the active VH and VL domains, each domain is linked to an antigen binding domain to a tumor antigen. As such, the protease cleaved product can bind to tumor cells and recruit T cells to the tumor site. In some embodiments, the first TTA and the second TTA are the same tumor antigen. In other embodiments, the first TTA and the second TTA are different tumor antigens.

The prodrug of “construct 3” is similar to “construct 2” but lacks a domain linker between the inactive VH domain and the CH3-hole polypeptide of the first Fc polypeptide, and a domain linker between the inactive VL domain and the CH3-knob polypeptide of the second Fc polypeptide.

Also provided herein is a prodrug construct (e.g., “construct 4”) that includes a first Fc polypeptide comprising a CH2-CH3-KIH polypeptide and an antigen binding domain (ABD) that can bind a target tumor antigen (TTA); and a second Fc polypeptide comprising a CH2-CH3-KIH polypeptide, a first pseudo Fv domain, a second pseudo Fv domain, and second antigen binding domain that can bind a target tumor antigen (TTA), and a third antigen binding domain. that can bind a target tumor antigen (TTA). In some cases the first Fc polypeptide contains the CH3-hole and the second contains the CH3-knob. In some embodiments, the first, second, and/or third antigen binding domain can bind the same tumor antigen. In other embodiments, the first, second, and/or third antigen binding domain are different tumor antigens. The first and second antigen binding domain can bind the same tumor antigen. The first and second antigen binding domain can bind different tumor antigens. The first and third antigen binding domain can bind the same tumor antigen. The first and third antigen binding domain can bind different tumor antigens. The second and third antigen binding domain can bind the same tumor antigen. The second and third antigen binding domain can bind different tumor antigens.

In the “construct 4” embodiments, the first Fc polypeptide (from N-terminal to C-terminal) includes: a first antigen binding domain for a TTA linked to a CH2-CH3-KIH polypeptide; and the second Fc polypeptide (from N-terminal to C-terminal) includes: a second antigen binding domain for a TTA linked via a domain linker to an active VH domain that is attached via a cleavable linker to an inactive VL domain that is linked to a CH2-CH3-KIH polypeptide that is linked via a cleavable linker to a third antigen binding domain for a TTA that is linked via a domain linker to an active VL domain that is linked via cleavable linker to an inactive VH domain. In some cases, the first Fc polypeptide contains the CH3-hole and the second contains the CH3-knob.

In the “construct 5” embodiments, the first Fc polypeptide (from N-terminal to C-terminal) includes: a first antigen binding domain for a TTA linked to a CH2-CH3-KIH polypeptide that is linked via a cleavable linker to a second antigen binding domain that is linked via a domain linker to an active VL domain that is linked via a cleavable linker to an inactive VH domain; and the second Fc polypeptide (from N-terminal to C-terminal) includes: a third antigen binding domain for a TTA linked via a domain linker to an active VH domain that is linked via a cleavable linker to an inactive VL domain that is linked to a CH2-CH3-KIH polypeptide. In some cases, the first Fc polypeptide contains the CH3-hole and the second contains the CH3-knob. In some embodiments, the first, second, and/or third antigen binding domain can bind the same tumor antigen. In other embodiments, the first, second, and/or third antigen binding domain are different tumor antigens. The first and second antigen binding domain can bind the same tumor antigen. The first and second antigen binding domain can bind different tumor antigens. The first and third antigen binding domain can bind the same tumor antigen. The first and third antigen binding domain can bind different tumor antigens. The second and third antigen binding domain can bind the same tumor antigen. The second and third antigen binding domain can bind different tumor antigens.

Provided herein is a prodrug construct (e.g., “construct 6”) that includes a first Fc polypeptide comprising a CH2-CH3-KIH polypeptide and first pseudo Fv domain that comprises a variable heavy domain and a variable light domain with standard framework regions and “inert” or “dummy” CDRs; and a second Fc polypeptide comprising a CH2-CH3-KIH polypeptide, an antigen binding domain (ABD) that can bind a target tumor antigen (TTA), and CD3 binding domain in a constrained format wherein the linker between the variable heavy and light domains that traditionally form an Fv is too short to allow the two domains to bind each other. In some embodiments, the constrained active Fv domain is covalently attached to the CH2-CH3-KIH polypeptide via a cleavable linker, and the first pseudo Fv domain is covalently attached to the CH2-CH3-KIH polypeptide via a cleavable linker. In some cases, the first Fc polypeptide contains the CH3-hole and the second contains the CH3-knob. In some embodiments, the cleavable linkers can be recognized by the same protease. In other embodiments, the cleavable linkers can be recognized by different proteases.

In the “construct 6” embodiments, the second Fc polypeptide (from N-terminal to C-terminal) includes: a first antigen binding domain for a TTA linked via a domain linker to a constrained active Fv domain (e.g., an active variable heavy chain linked via a constrained, non-cleavable linker to an active variable light chain, or an active variable light chain linked via a constrained, non-cleavable linker to an active variable heavy chain) that is linked via a cleavable linker to a CH2-CH3-KIH polypeptide; and the first Fc polypeptide (from N-terminal to C-terminal) includes: a pseudo Fv domain (e.g., an inactive variable light domain that is linked via a non-cleavable linker to an inactive variable heavy domain, or an inactive variable heavy domain that is linked via a non-cleavable linker to an inactive variable light domain,) that is linked via a cleavable linker or a non-cleavable linker to a CH2-CH3-KIH polypeptide. In some cases, the first Fc polypeptide contains the CH3-hole and the second contains the CH3-knob.

Provided herein is another prodrug construct (e.g., “construct 7”) that is similar to “construct 6”. Exemplary embodiments of construct 7 include a second Fc polypeptide comprising a CH2-CH3-KIH polypeptide, a first antigen binding domain (ABD) that can bind a target tumor antigen (TTA), a second antigen binding domain (ABD) that can bind a target tumor antigen (TTA), and CD3 binding domain in a constrained format wherein the linker between the variable heavy and light domains that traditionally form an Fv is too short to allow the two domains to bind each other; and a first Fc polypeptide comprising a CH2-CH3-KIH polypeptide and first pseudo Fv domain. In some cases, the first Fc polypeptide contains the CH3-hole and the second contains the CH3-knob. In some instances, the first and second antigen binding domain can bind the same tumor antigen. In other instances, the first and second antigen binding domain can bind different tumor antigens.

In the “construct 7” embodiments, the second Fc polypeptide (from N-terminal to C-terminal) includes: a first antigen binding domain for a TTA linked via a domain linker to a constrained active Fv domain (e.g., an active variable heavy chain linked via a constrained, non-cleavable linker to an active variable light chain, or an active variable light chain linked via a constrained, non-cleavable linker to an active variable heavy chain) that is linked via a domain linker to a second antigen binding domain that is linked via a cleavable linker to a CH2-CH3-KIH polypeptide; and the first Fc polypeptide (from N-terminal to C-terminal) includes: a pseudo Fv domain (e.g., an inactive variable light domain that is linked via a non-cleavable linker to an inactive variable heavy domain, or an inactive variable heavy domain that is linked via a non-cleavable linker to an inactive variable light domain,) that is linked via a cleavable or non-cleavable linker to a CH2-CH3-KIH polypeptide. In some cases, the first Fc polypeptide contains the CH3-hole and the second contains the CH3-knob. In some embodiments, the cleavable linker adjacent to the CH2-CH3-knob polypeptide is the same cleavable linker adjacent to the CH2-CH3-hole polypeptide. In other embodiments, the cleavable linkers are different.

Provided herein is a homodimeric Fc prodrug construct (e.g., “construct 8”). Exemplary embodiments of construct 8 include a first Fc polypeptide comprising a first antigen binding domain (ABD) that can bind a target tumor antigen (TTA), a first CD3 binding domain in a constrained format wherein the linker between the variable heavy and light domains that traditionally form an Fv is too short to allow the two domains to bind each other, a second antigen binding domain (ABD) that can bind a target tumor antigen (TTA) a first pseudo Fv domain, and a CH2-CH3 polypeptide; and a second Fc polypeptide comprising a third antigen binding domain (ABD) that can bind a target tumor antigen (TTA), a second CD3 binding domain in a constrained format wherein the linker between the variable heavy and light domains that traditionally form an Fv is too short to allow the two domains to bind each other, a fourth antigen binding domain (ABD) that can bind a target tumor antigen (TTA) a second pseudo Fv domain, and a second CH2-CH3 polypeptide. In some instances, the first and second antigen binding domain can bind the same tumor antigen. In other instances, the first and second antigen binding domain can bind different tumor antigens. In some instances, the third and fourth antigen binding domain can bind the same tumor antigen. In other instances, the third and fourth antigen binding domain can bind different tumor antigens. In some instances, the first, second, third and/or fourth antigen binding domain can bind the same tumor antigen.

In the “construct 8” embodiments, the first Fc polypeptide (from N-terminal to C-terminal) includes: a first antigen binding domain for a TTA linked via a domain linker to a first constrained active CD3 Fv domain (e.g., an active variable heavy chain linked via a constrained, non-cleavable linker to an active variable light chain, or an active variable light chain linked via a constrained, non-cleavable linker to an active variable heavy chain) that is linked a second antigen binding domain that is linked via a cleavable linker to a first pseudo CD3 Fv domain (e.g., an inactive variable light domain that is linked via a non-cleavable linker to an inactive variable heavy domain, or an inactive variable heavy domain that is linked via a non-cleavable linker to an inactive variable light domain) linked to a CH2-CH3 polypeptide; and the second Fc polypeptide (from N-terminal to C-terminal) includes: a third antigen binding domain for a TTA linked via a domain linker to a second constrained active CD3 Fv domain (e.g., an active variable heavy chain linked via a constrained, non-cleavable linker to an active variable light chain, or an active variable light chain linked via a constrained, non-cleavable linker to an active variable heavy chain) that is linked a fourth antigen binding domain that is linked via a cleavable linker to a second pseudo CD3 Fv domain (e.g., an inactive variable light domain that is linked via a non-cleavable linker to an inactive variable heavy domain, or an inactive variable heavy domain that is linked via a non-cleavable linker to an inactive variable light domain) linked to a CH2-CH3 polypeptide. In some embodiments, the first Fc polypeptide and the second Fc polypeptide of the homodimer are the same.

Provided herein is another heterodimeric Fc prodrug construct (e.g., construct 9″). Exemplary embodiments of construct 9 include a first Fc polypeptide comprising a CH2-CH3-KIH polypeptide; and a second Fc polypeptide comprising a first antigen binding domain (ABD) that can bind a target tumor antigen (TTA), a first CD3 binding domain in a constrained format wherein the linker between the variable heavy and light domains that traditionally form an Fv is too short to allow the two domains to bind each other, a second antigen binding domain (ABD) that can bind a target tumor antigen (TTA) a first pseudo Fv domain, and a CH2-CH3-KIH polypeptide. In some instances, the first and second antigen binding domain can bind the same tumor antigen. In other instances, the first and second antigen binding domain can bind different tumor antigens. In some embodiments, the first Fc polypeptide comprises an Fc-hole domain and the second Fc polypeptide comprises an Fc-knob domain. In some embodiments, the first Fc polypeptide comprises an Fc-knob domain and the second Fc polypeptide comprises an Fc-hole domain.

In the “construct 9” embodiments, the first Fc polypeptide (from N-terminal to C-terminal) includes: a domain linker (hinge linker)-CH2-CH3-KIH polypeptide; and the second Fc polypeptide (from N-terminal to C-terminal) includes: a first antigen binding domain for a TTA linked via a domain linker to a constrained active CD3 Fv domain (e.g., an active variable heavy chain linked via a constrained, non-cleavable linker to an active variable light chain, or an active variable light chain linked via a constrained, non-cleavable linker to an active variable heavy chain) that is linked a second antigen binding domain that is linked via a cleavable linker to a pseudo CD3 Fv domain (e.g., an inactive variable light domain that is linked via a non-cleavable linker to an inactive variable heavy domain, or an inactive variable heavy domain that is linked via a non-cleavable linker to an inactive variable light domain) linked to a CH2-CH3-KIH polypeptide.

Provided herein is a dimeric Fc prodrug construct (e.g., “construct 10” or homodimeric Fc construct). Exemplary embodiments of construct 10 include a first Fc polypeptide comprising a first antigen binding domain (ABD) that can bind a target tumor antigen (TTA), a first CD3 binding domain in a constrained format wherein the linker between the variable heavy and light domains that traditionally form an Fv is too short to allow the two domains to bind each other, a second antigen binding domain (ABD) that can bind a target tumor antigen (TTA) a first pseudo Fv domain, and a first CH2-CH3 polypeptide; and a second Fc polypeptide comprising a third antigen binding domain (ABD) that can bind a target tumor antigen (TTA), a second CD3 binding domain in a constrained format wherein the linker between the variable heavy and light domains that traditionally form an Fv is too short to allow the two domains to bind each other, a fourth antigen binding domain (ABD) that can bind a target tumor antigen (TTA) a second pseudo Fv domain, and a second CH2-CH3 polypeptide. In some instances, the first and second antigen binding domain can bind the same tumor antigen. In other instances, the first and second antigen binding domain can bind different tumor antigens. In some instances, the third and fourth antigen binding domain can bind the same tumor antigen. In other instances, the third and fourth antigen binding domain can bind different tumor antigens. In some instances, the first, second, third and/or fourth antigen binding domain can bind the same tumor antigen. In other instances, the first, second, third and/or fourth antigen binding domain can bind different tumor antigens.

In the “construct 10” embodiments, the first Fc polypeptide (from N-terminal to C-terminal) includes: a first antigen binding domain for a TTA linked via a domain linker to a first constrained active CD3 Fv domain (e.g., an active variable heavy chain linked via a constrained, non-cleavable linker to an active variable light chain, or an active variable light chain linked via a constrained, non-cleavable linker to an active variable heavy chain) that is linked a second antigen binding domain that is linked via a cleavable linker to a first pseudo CD3 Fv domain (e.g., an inactive variable light domain that is linked via a non-cleavable linker to an inactive variable heavy domain, or an inactive variable heavy domain that is linked via a non-cleavable linker to an inactive variable light domain) linked to a first CH2-CH3 polypeptide; and the second Fc polypeptide (from N-terminal to C-terminal) includes: a third antigen binding domain for a TTA linked via a domain linker to a second constrained active CD3 Fv domain (e.g., an active variable heavy chain linked via a constrained, non-cleavable linker to an active variable light chain, or an active variable light chain linked via a constrained, non-cleavable linker to an active variable heavy chain) that is linked a fourth antigen binding domain that is linked via a cleavable linker to a second pseudo CD3 Fv domain (e.g., an inactive variable light domain that is linked via a non-cleavable linker to an inactive variable heavy domain, or an inactive variable heavy domain that is linked via a non-cleavable linker to an inactive variable light domain) linked to a second CH2-CH3 polypeptide. In some embodiments, the cleavable linker adjacent to the first CH2-CH3 polypeptide is the same cleavable linker adjacent to the second CH2-CH3 polypeptide. In other embodiments, the cleavable linkers are different. In some embodiments, the first Fc polypeptide comprises an Fc-hole domain and the second Fc polypeptide comprises an Fc-knob domain. In some embodiments, the first Fc polypeptide comprises an Fc-knob domain and the second Fc polypeptide comprises an Fc-hole domain.

### II. Definitions

In order that the application may be more completely understood, several definitions are set forth below. Such definitions are meant to encompass grammatical equivalents.

The term “COBRA™” and “COnditional Bispecific Redirected Activation” refers to a bispecific conditionally effective protein that has a number of functional protein domains. In some embodiments, one of the functional domain is an antigen binding domain (ABD) that binds a target tumor antigen (TTA). In certain embodiments, another domain is an ABD that binds to a T cell antigen under certain conditions. The T cell antigen includes but is not limited to CD3. The term “hemi-COBRA™” refers to a conditionally effective protein that can bind a T cell antigen when a variable heavy chain of a hemi-COBRA can associate to a variable light chain of another hemi-COBRA™ (a complementary hemi-COBRA™) due to innate self-assembly when concentrated on the surface of a target expressing cell.

By “amino acid” and “amino acid identity” as used herein is meant one of the 20 naturally occurring amino acids or any non-natural analogues that may be present at a specific, defined position. In many embodiments, “amino acid” means one of the 20 naturally occurring amino acids. By “protein” herein is meant at least two covalently attached amino acids, which includes proteins, polypeptides, oligopeptides and peptides.

By “amino acid modification” herein is meant an amino acid substitution, insertion, and/or deletion in a polypeptide sequence or an alteration to a moiety chemically linked to a protein. For example, a modification may be an altered carbohydrate or PEG structure attached to a protein. For clarity, unless otherwise noted, the amino acid modification is always to an amino acid coded for by DNA, e.g., the 20 amino acids that have codons in DNA and RNA. The preferred amino acid modification herein is a substitution.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For clarity, a protein which has been engineered to change the nucleic acid coding sequence but not change the starting amino acid (for example, exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence.

By “amino acid deletion” or “deletion” as used herein is meant the removal of an amino acid sequence at a particular position in a parent polypeptide sequence.

The polypeptides of the invention specifically bind to CD3 and target tumor antigens (TTAs) such as target cell receptors, as outlined herein. “Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore assay or Octet as is known in the art.

By “parent polypeptide” or “precursor polypeptide” (including Fc parent or precursors) as used herein is meant a polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent Fc polypeptide” as used herein is meant an unmodified Fc polypeptide that is modified to generate a variant, generally a human IgG Fc domain as defined herein and by “parent antibody” as used herein is meant an unmodified antibody that is modified to generate a variant antibody.

By “position” as used herein is meant a location in the sequence of a protein. Positions may be numbered sequentially, or according to an established format, for example the EU index for antibody numbering.

By “target antigen” as used herein is meant the molecule that is bound specifically by the variable region of a given antibody. A target antigen may be a protein, carbohydrate, lipid, or other chemical compound. A range of suitable exemplary target antigens are described herein.

By “target cell” as used herein is meant a cell that expresses a target antigen.

By “Fv” or “Fv domain” or “Fv region” as used herein is meant a polypeptide that comprises the VL and VH domains of an antigen binding domain, generally from an antibody. Fv domains usually form an “antigen binding domain” or “ABD” as discussed herein, if they contain active VH and VL domains (although in some cases, an Fv containing a constrained linker As discussed below, Fv domains can be organized in a number of ways in the present invention, and can be “active” or “inactive”, such as in a scFv format, a constrained Fv format, a pseudo Fv format, etc. It should be understood that in the present invention, in some cases an Fv domain is made up of a VH and VL domain on a single polypeptide chain, such as shown FIGS. 8 and 9, but with a constrained linker such that an intramolecular ABD cannot be formed. In these embodiments, it is after cleavage that two active ABDs are formed. In some cases an Fv domain is made up of a VH and a VL domain, one of which is inert, such that only after cleavage is an intermolecular ABD formed.

By “variable domain” herein is meant the region of an immunoglobulin that comprises one or more Ig domains substantially encoded by any of the Vic Vii, and/or VH genes that make up the kappa, lambda, and heavy chain immunoglobulin genetic loci respectively. Each VH and VL is composed of three hypervariable regions (“complementary determining regions,” “CDRs”) and four “framework regions”, or “FRs”, arranged from amino-terminus to carboxy-terminus in the following order: FR1-CDR1-FR2-CDR2-FR3-CDR3-FR4. Thus, the VH domain has the structure vhFR1-vhCDR1-vhFR2-vhCDR2-vhFR3-vhCDR3-vhFR4 and the VL domain has the structure vlFR1-vlCDR1-vlFR2-vlCDR2-vlFR3-vlCDR3-vlFR4. As is more fully described herein, the vhFR regions and the vlFR regions self-assemble to form Fv domains. In general, in the prodrug formats of the invention, there are “constrained Fv domains” wherein the VH and VL domains cannot self-associate, and “pseudo Fv domains” for which the CDRs do not form functional (active) antigen binding domains when self-associated.

The hypervariable regions confer antigen binding specificity and generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3).

A useful comparison of CDR numbering is as below, see Lafranc et al., Dev. Comp. Immunol. 27(1):55-77 (2003):

TABLE 1

| | Kabat + | | | | | |
|---|---|---|---|---|---|---|
| | Chothia | IMGT | Kabat | AbM | Chothia | Contact |
| vhCDR1 | 26-35 | 27-38 | 31-35 | 26-35 | 26-32 | 30-35 |
| vhCDR2 | 50-65 | 56-65 | 50-65 | 50-58 | 52-56 | 47-58 |
| vhCDR3 | 95-102 | 105-117 | 95-102 | 95-102 | 95-102 | 93-101 |
| vlCDR1 | 24-34 | 27-38 | 24-34 | 24-34 | 24-34 | 30-36 |
| vlCDR2 | 50-56 | 56-65 | 50-56 | 50-56 | 50-56 | 46-55 |
| vlCDR3 | 89-97 | 105-117 | 89-97 | 89-97 | 89-97 | 89-96 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The present invention provides a large number of different CDR sets. In this case, a “full CDR set” comprises the three variable light and three variable heavy CDRs, e.g. a vlCDR1, vlCDR2, vlCDR3, vhCDR1, vhCDR2 and vhCDR3. As will be appreciated by those in the art, each set of CDRs, the VH and VL CDRs, can bind to antigens, both individually and as a set. For example, in constrained Fv domains, the vhCDRs can bind, for example to CD3 and the vlCDRs can bind to CD3, but in the constrained format they cannot bind to CD3.

These CDRs can be part of a larger variable light or variable heavy domain, respectfully. In addition, as more fully outlined herein, the variable heavy and variable light domains can be on separate polypeptide chains or on a single polypeptide chain in the case of scFv sequences.

The CDRs contribute to the formation of the antigen-binding, or more specifically, epitope binding sites. “Epitope” refers to a determinant that interacts with a specific antigen binding site in the variable regions known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

The variable heavy and variable light domains of the invention can be “active” or “inactive”.

As used herein, “inactive VH” (“iVH”) and “inactive VL” (“iVL”) refer to components of a pseudo Fv domain, which, when paired with their cognate VL or VH partners, respectively; form a resulting VH/VL pair that does not specifically bind to the antigen to which the “active” VH or “active” VL would bind were it bound to an analogous VL or VH, which was not “inactive”. Exemplary “inactive VH” and “inactive VL” domains are formed by mutation of a wild type VH or VL sequence. Exemplary mutations are within CDR1, CDR2 or CDR3 of VH or VL. An exemplary mutation includes placing a domain linker within CDR2, thereby forming an “inactive VH” or “inactive VL” domain. In contrast, an “active VH” (aVH) or “active VL” (aVL) is one that, upon pairing with its “active” cognate partner, i.e., VL or VH, respectively, is capable of specifically binding to its target antigen.

In contrast, as used herein, the term “active” refers to a CD3 binding domain that is capable of specifically binding to CD3. This term is used in two contexts: (a) when referring to a single member of an Fv binding pair (i.e., VH or VL), which is of a sequence capable of pairing with its cognate partner and specifically binding to CD3; and (b) the pair of cognates (i.e., VH and VL) of a sequence capable of specifically binding to CD3. An exemplary “active” VH, VL or VH/VL pair is a wild type or parent sequence.

“CD-x” refers to a cluster of differentiation (CD) protein. In exemplary embodiments, CD-x is selected from those CD proteins having a role in the recruitment or activation of T-cells in a subject to whom a polypeptide construct of the invention has been administered. In an exemplary embodiment, CD-x is CD3.

The term “binding domain” characterizes, in connection with the present invention, a domain which (specifically) binds to/interacts with/recognizes a given target epitope or a given target site on the target molecules (antigens), for example: EGFR and CD3, respectively. The structure and function of the target antigen binding domain (recognizing EGFR), and preferably also the structure and/or function of the CD3 binding domain (recognizing CD3), is/are based on the structure and/or function of an antibody, e.g. of a full-length or whole immunoglobulin molecule, including sdABDs. According to the invention, the target antigen binding domain is generally characterized by the presence of three CDRs that bind the target tumor antigen (generally referred to in the art as variable heavy domains, although no corresponding light chain CDRs are present). Alternatively, ABDs to TTAs can include three light chain CDRs (i.e., CDR1, CDR2 and CDR3 of the VL region) and/or three heavy chain CDRs (i.e., CDR1, CDR2 and CDR3 of the VH region). The CD3 binding domain preferably also comprises at least the minimum structural requirements of an antibody which allow for the target binding. More preferably, the CD3 binding domain comprises at least three light chain CDRs (i.e., CDR1, CDR2 and CDR3 of the VL region) and/or three heavy chain CDRs (i.e., CDR1, CDR2 and CDR3 of the VH region). It is envisaged that in exemplary embodiments the target antigen and/or CD3 binding domain is produced by or obtainable by phage-display or library screening methods.

By “domain” as used herein is meant a protein sequence with a function, as outlined herein. Domains of the invention include tumor target antigen binding domains (TTA domains), variable heavy domains, variable light domains, linker domains, and half life extension domains.

By “domain linker” herein is meant an amino acid sequence that joins two domains as outlined herein. Domain linkers can be cleavable linkers, constrained cleavable linkers, non-cleavable linkers, constrained non-cleavable linkers, scFv linkers, etc.

By “hinge linker” herein is meant an amino acid sequence that joins a domain to a hinge region of a Fc domain as outlined herein. Hinge linkers can be cleavable linkers, constrained cleavable linkers, non-cleavable linkers, constrained non-cleavable linkers, scFv linkers, etc.

By “cleavable linker” (“CL”) herein is meant an amino acid sequence that can be cleaved by a protease, preferably a human protease in a disease tissue as outlined herein. Cleavable linkers generally are at least 3 amino acids in length, with from 4, 5, 6, 7, 8, 9, 10, 11, 12, 13, 14, 15 or more amino acids finding use in the invention, depending on the required flexibility.

By “non cleavable linker” (“NCL”) herein is meant an amino acid sequence that cannot be cleaved by a human protease under normal physiological conditions.

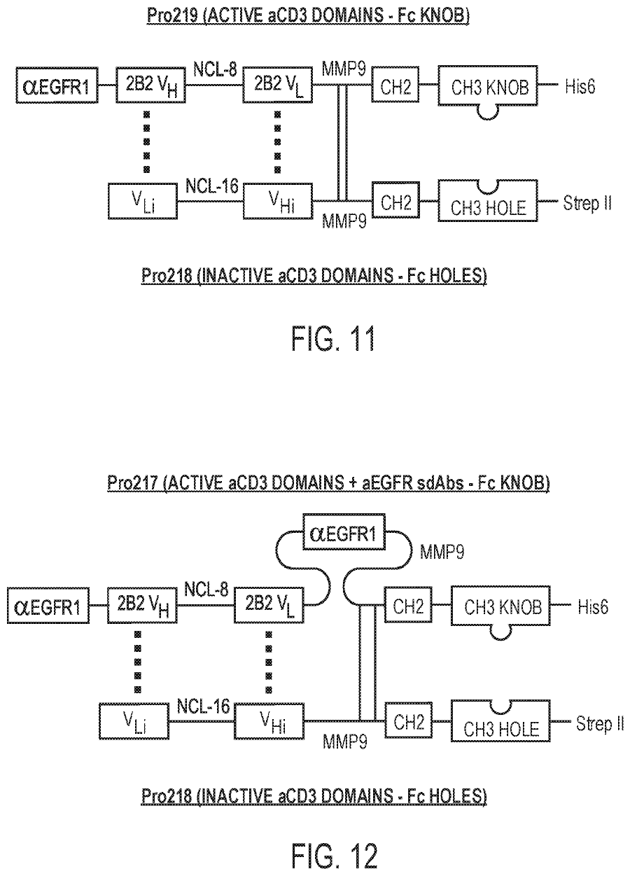

By “cleavable constrained linker” or “constrained cleavable linker” (“CCL”) herein is meant a short polypeptide that contains a protease cleavage site (as defined herein) that joins two domains as outlined herein in such a manner that the two domains cannot significantly interact with each other until after they reside on different polypeptide chains, e.g., after cleavage. When the CCL joins a VH and a VL domain as defined herein, the VH and VL cannot self-assemble to form a functional Fv prior to cleavage due to steric constraints in an intramolecular way. Upon cleavage by the relevant protease, the VH and VL can assemble to form an active antigen binding domain in a intermolecular way. In general, CCLs are less than 10 amino acids in length, with 9, 8, 7, 6, 5 and 4 amino acids finding use in the invention. In general, protease cleavage sites generally are at least 4+ amino acids in length to confer sufficient specificity, as shown in FIG. 11A, FIG. 11B, and FIG. 11C.

By “non-cleavable constrained linker” (“NCCL”) or “constrained non-cleavable linker” (“CNCL”) herein is meant a short polypeptide that that joins two domains as outlined herein in such a manner that the two domains cannot significantly interact with each other, and that is not significantly cleaved by human proteases under physiological conditions.

By “constrained Fv domain” herein is meant an Fv domain that comprises an active variable heavy domain and an active variable light domain, linked covalently with a constrained linker as outlined herein, in such a way that the active heavy and light variable domains cannot intramolecularly interact to form an active Fv that will bind an antigen such as CD3. Thus, a constrained Fv domain is one that is similar to an scFv but is not able to bind an antigen due to the presence of a constrained linker.

By “pseudo Fv domain” herein is meant a domain that comprises (i) a pseudo or inactive variable heavy domain and a pseudo or inactive variable light domain, (ii) a pseudo or inactive variable heavy domain and an active variable light domain, or (iii) an active variable heavy domain and a pseudo or inactive variable light domain, linked using a domain linker (which can be cleavable, constrained, non-cleavable, non-constrained, etc.). The VHi and VLi domains of a pseudo Fv domain do not bind to a human antigen when either associated with each other (VHi/VLi) or when associated with an active VH or VL; thus VHi/VLi, VHi/VL and VLi/VH Fv domains do not appreciably bind to a human protein, such that these domains are inert in the human body.

By “single chain Fv” or “scFv” herein is meant a variable heavy (VH) domain covalently attached to a variable light (VL) domain, generally using a scFv linker as discussed herein, to form a scFv or scFv domain. A scFv domain can be in either orientation from N- to C-terminus (VH-linker-VL or VL-linker-VH).

By “single domain Fv”, “sdFv”, “single domain antibody” or “sdABD” herein is meant an antigen binding domain that only has three CDRs, generally based on camelid antibody technology. See: Protein Engineering 9(7):1129-35 (1994); Rev Mol Biotech 74:277-302 (2001); Ann Rev Biochem 82:775-97 (2013).

By “protease cleavage site” refers to the amino acid sequence recognized and cleaved by a protease. Suitable protease cleavage sites are outlined below.

As used herein, “protease cleavage domain” refers to the peptide sequence incorporating the “protease cleavage site” and any linkers between individual protease cleavage sites and between the protease cleavage site(s) and the other functional components of the constructs of the invention (e.g., VH, VL, VHi, VLi, target antigen binding domain(s), half-life extension domain, etc.).

By “Fc” or “Fc region” or “Fc domain” as used herein is meant the polypeptide comprising the constant region of an antibody excluding the first constant region immunoglobulin domain. For IgG, the Fc domain comprises immunoglobulin domains Cγ2 and Cγ3 (CH2 and CH3), and optionally all or part of the hinge region between Cγ1 (CH1) and Cγ2 (CH2). In the EU numbering for human IgG1, the CH2-CH3 domain comprises amino acids 231 to 447, and the hinge is 216 to 230. Thus, the definition of “Fc domain” includes both amino acids 231-447 (CH2-CH3) or 216-447 (hinge domain-CH2-CH3).

### III. Proteins of the Invention

The proteins of the invention have a number of different components, generally referred to herein as domains, that are linked together in a variety of ways. Some of the domains are binding domains, that each bind to a target antigen (e.g., a TTA or CD3, for example). As they bind to more than one antigen, they are referred to herein as “multispecific”; for example, a prodrug construct of the invention may bind to a TTA and CD3, and thus are “bispecific,” as shown in FIG. 1. A protein of the present invention can also have higher specificities; for example, if the first antigen binding domain binds to EGFR, the second antigen binding domain binds to EpCAM and there is an anti-CD3 binding domain, this would be a “trispecific” molecule.

The proteins of the invention can include CD3 antigen binding domains arranged in a variety of ways as outlined herein, tumor target antigen binding domains, half-life extension domains, linkers, etc.

In some embodiments, a first protein comprises a first tumor target antigen binding domain and a second protein comprises a second tumor target antigen binding domain such that the first tumor target antigen binding domain and second tumor target antigen binding domain bind to the same tumor target antigen. In certain instances, the first tumor target antigen domain and second tumor target antigen domain bind different epitopes, regions, or portions of the same tumor target antigen. In some instances, the first tumor target antigen domain and second tumor target antigen domain bind different tumor target antigens.

The proteins of the invention can be produced by co-expression in a cell and co-purification to obtain a complementary pair of proteins that can bind to CD3 and a tumor target antigen. In some embodiments, each of the complementary pair of proteins are purified separately. In some embodiments, each of the complementary pair of proteins are purified simultaneously or concomitantly.

In some embodiments, an expression vector comprises a nucleic acid sequence encoding one protein of the complementary pair of proteins and a nucleic acid sequence encoding the other protein of the complementary pair of proteins. In some embodiments, a host cell comprises such an expression vector. In some instances, such a host cell can be cultured under suitable conditions in a culture media to produce the proteins. In some embodiments, the host cell is cultured under suitable conditions to secrete the proteins described herein into the culture media. In certain embodiments, the culture media comprising the secreted proteins of the invention is purified to obtain proteins of the complementary pair of proteins. Useful methods of purification include, but are not limited to, protein A chromatography, protein G chromatography, heparin binding, reverse phase chromatography, HIC chromatography, CHT chromatography affinity chromatography, anion exchange chromatography, cation exchange chromatography, size exclusion chromatography, and the like.

### A. CD3 Antigen Binding Domains

The specificity of the response of T cells is mediated by the recognition of antigen (displayed in context of a major histocompatibility complex, MHC) by the T cell receptor complex. As part of the T cell receptor complex, CD3 is a protein complex that includes a CD3γ (gamma) chain, a CD3δ (delta) chain, and two CDRε (epsilon) chains which are present on the cell surface. CD3 associates with the α (alpha) and β (beta) chains of the T cell receptor (TCR) as well as and CD-ζ (zeta) altogether to comprise the T cell receptor complex. Clustering of CD3 on T cells, such as by Fv domains that bind to CD3 leads to T cell activation similar to the engagement of the T cell receptor but independent of its clone-typical specificity.

However, as is known in the art, CD3 activation can cause a number of toxic side effects, and accordingly the present invention is directed to providing active CD3 binding of the polypeptides of the invention only in the presence of tumor cells, where specific proteases are found, that then cleave the prodrug polypeptides of the invention to provide an active CD3 binding domain. Thus, in the present invention, binding of an anti-CD3 Fv domain to CD3 is regulated by a protease cleavage domain which restricts binding of the CD3 Fv domain to CD3 only in the microenvironment of a diseased cell or tissue with elevated levels of proteases, for example in a tumor microenvironment as is described herein. Accordingly, the present invention provides two sets of VH and VL domains, an active set (VH and VL) and an inactive set (VHi and VLi) with all four being present in the prodrug construct(s). The construct is formatted such that the VH and VL set cannot self-associate, but rather associates with an inactive partner, e.g. VHi and VL and VLi and VH as is shown herein.

There are a number of suitable active CDR sets, and/or VH and VL domains, that are known in the art that find use in the present invention. For example, the CDRs and/or VH and VL domains are derived from known anti-CD3 antibodies, such as, for example, muromonab-CD3 (OKT3), otelixizumab (TRX4), teplizumab (MGA031), visilizumab (Nuvion), SP34 or I2C, TR-66 or X35-3, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31.

In some embodiments, the VH and VL sequences that form an active Fv domain that binds to human CD3 are shown in FIG. 2A-2B as CD3 VH (SEQ ID NO:186) and CD3 VL (SEQ ID NO:170).

The inactive VHi and VLi domains contain “regular” framework regions (FRs) that allow association, such that an inactive variable domain will associate with an active variable domain, rendering the pair inactive, e.g., unable to bind CD3. In one embodiment, the VHi and VLi that form inactive Fv domains when one or both of the inactive domains are present in a complementary construct pair. In one embodiment, the VHi and VLi that form inactive Fv domains when one or both of the inactive domains are present are shown in FIG. 2A-2B as VHi (SEQ ID NO:190) and VLi (SEQ ID NO:174). In one embodiment, the VHi2 and VLi2 that form inactive Fv domains when one or both of the inactive domains are present are shown in FIG. 2A-2B as VHi2 (SEQ ID NO:194) and VLi2 (SEQ ID NO:178). In one embodiment, the VHGL4 and VLiGL that form inactive Fv domains when one or both of the inactive domains are present are shown in FIG. 2A-2B as VHiGL4 (SEQ ID NO:198) and VLiGL (SEQ ID NO:182).

In some embodiments, the inactive VHi domain comprises one or more, e.g., 1, 2, 3, 4, 5, 6, 7, 8, 9, or more, amino acid modifications (e.g., amino acid insertions, deletions, or substitutions) that when paired with an active VL domain renders the paired VHi-VL domain unable to bind the target antigen. In other embodiments, the inactive VLi domain comprises one or more, e.g., 1, 2, 3, 4, 5, 6, 7, 8, 9, or more, amino acid modifications (e.g., amino acid insertions, deletions, or substitutions) that when paired with an active VH domain renders the paired VH-VLi unable to bind the target antigen.

As will be appreciated by those in the art, there are a number of “inactive” variable domains that find use in the invention. Basically, any variable domain with human framework regions that allows self-assembly with another variable domain, no matter what amino acids are in the CDR location in the variable region can be used. For clarity, the inactive domains are said to include CDRs, although technically the inactive variable domains do not confer binding capabilities.

In some cases, the inactive domains can be engineered to promote selective binding in the prodrug format, to encourage formation of intramolecular VHi-VL and VH-VLi domains prior to cleavage (over, for example, intermolecular pair formation). See, for example, Igawa et al., Protein Eng. Des. Selection 23(8):667-677 (2010), hereby expressly incorporated by reference in its entirety and specifically for the interface residue amino acid substitutions.

In one aspect, the polypeptide constructs described herein comprise a domain which specifically binds to CD3 when activated by a protease. In one aspect, the polypeptide constructs described herein comprise two or more domains which when activated by a protease specifically bind to human CD3. In some embodiments, the polypeptide constructs described herein comprise two or more domains which when activated by a protease which specifically binds to CDR. In some embodiments, the polypeptide constructs described herein comprise two or more domains which when activated by a protease specifically bind to CD3c.

In some embodiments, the protease cleavage site is between the anti-CD3 active VH and inactive VL domains on a first monomer and keeps them from folding and binding to CD3 on a T cell. In some embodiments, the protease cleavage site is between the anti-CD3 inactive VH and active VL domains on a second monomer and keeps them from folding and binding to CD3 on a T cell. Once protease cleavage sites are cleaved by a protease present at the target cell, the anti-CD3 active VH domain of the first monomer and the anti-CD3 active VL domain of the second monomer are able to bind to CD3 on a T cell.

In certain embodiments, the CD3 binding domain of the polypeptide constructs described herein exhibit not only potent CD3 binding affinities with human CD3, but show also excellent cross reactivity with the respective cynomolgus monkey CD3 proteins. In some instances, the CD3 binding domain of the polypeptide constructs is cross-reactive with CD3 from cynomolgus monkey. In certain instances, human:cynomolgous KD ratios for CD3 are between 5 and 0.2.

In some embodiments, the CD3 binding domain of the antigen binding protein can be any domain that binds to CD3 including but not limited to domains from a monoclonal antibody, a polyclonal antibody, a recombinant antibody, a human antibody, a humanized antibody. In some instances, it is beneficial for the CD3 binding domain to be derived from the same species in which the antigen binding protein will ultimately be used in. For example, for use in humans, it may be beneficial for the CD3 binding domain of the antigen binding protein to comprise human or humanized residues from the antigen binding domain of an antibody or antibody fragment.

Thus, in one aspect, the antigen-binding domain comprises a humanized or human binding domain. In one embodiment, the humanized or human anti-CD3 binding domain comprises one or more (e.g., all three) light chain complementary determining region 1 (LC CDR1), light chain complementary determining region 2 (LC CDR2), and light chain complementary determining region 3 (LC CDR3) of a humanized or human anti-CD3 binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized or human anti-CD3 binding domain described herein, e.g., a humanized or human anti-CD3 binding domain comprising one or more, e.g., all three, LC CDRs and one or more, e.g., all three, HC CDRs.

In some embodiments, the humanized or human anti-CD3 binding domain comprises a humanized or human light chain variable region specific to CD3 where the light chain variable region specific to CD3 comprises human or non-human light chain CDRs in a human light chain framework region. In certain instances, the light chain framework region is a λ (lambda) light chain framework. In other instances, the light chain framework region is a κ (kappa) light chain framework.

In some embodiments, one or more CD3 binding domains are humanized or fully human. In some embodiments, one or more activated CD3 binding domains have a KD binding of 1000 nM or less to CD3 on CD3 expressing cells. In some embodiments, one or more activated CD3 binding domains have a KD binding of 100 nM or less to CD3 on CD3 expressing cells. In some embodiments, one or more activated CD3 binding domains have a KD binding of 10 nM or less to CD3 on CD3 expressing cells. In some embodiments, one or more CD3 binding domains have cross-reactivity with cynomolgus CD3. In some embodiments, one or more CD3 binding domains comprise an amino acid sequence provided herein.

In some embodiments, the humanized or human anti-CD3 binding domain comprises a humanized or human heavy chain variable region specific to CD3 where the heavy chain variable region specific to CD3 comprises human or non-human heavy chain CDRs in a human heavy chain framework region.

In one embodiment, the anti-CD3 binding domain is an Fv comprising a light chain and a heavy chain of an amino acid sequence provided herein. In an embodiment, the anti-CD3 binding domain comprises: a light chain variable region comprising an amino acid sequence having at least one, two or three modifications (e.g., substitutions, insertions, and deletions) but not more than 30, 20 or 10 modifications (e.g., substitutions, insertions, and deletions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions, insertions, and deletions) but not more than 30, 20 or 10 modifications (e.g., substitution, insertions, and deletions s) of an amino acid sequence of a heavy chain variable region provided herein, or a sequence with 95-99% identity to an amino acid sequence provided herein. In one embodiment, the humanized or human anti-CD3 binding domain is a scFv, and a light chain variable region comprising an amino acid sequence described herein, is attached to a heavy chain variable region comprising an amino acid sequence described herein, via a scFv linker. The light chain variable region and heavy chain variable region of a scFv can be, e.g., in any of the following orientations: light chain variable region-scFv linker-heavy chain variable region or heavy chain variable region-scFv linker-light chain variable region.

In some embodiments, CD3 binding domain of an antigen binding protein has an affinity to CD3 on CD3-expressing cells with a KD of 1000 nM or less, 100 nM or less, 50 nM or less, 20 nM or less, 10 nM or less, 5 nM or less, 1 nM or less, or 0.5 nM or less. In some embodiments, the CD3 binding domain of an antigen binding protein has an affinity to CD3ε with a KD of 1000 nM or less, 100 nM or less, 50 nM or less, 20 nM or less, 10 nM or less, 5 nM or less, 1 nM or less, or 0.5 nM or less. In further embodiments, CD3 binding domain of an antigen binding protein has low affinity to CD3, i.e., about 100 nM or greater.

The affinity to bind to CD3 can be determined, for example, by the ability of the antigen binding protein itself or its CD3 binding domain to bind to CD3 coated on an assay plate; displayed on a microbial cell surface; in solution; etc., as is known in the art, generally using Biacore or Octet assays. The binding activity of the antigen binding protein itself or its CD3 binding domain of the present disclosure to CD3 can be assayed by immobilizing the ligand (e.g., human CD3) or the antigen binding protein itself or its CD3 binding domain, to a bead, substrate, cell, etc. Agents can be added in an appropriate buffer and the binding partners incubated for a period of time at a given temperature. After washes to remove unbound material, the bound protein can be released with, for example, SDS, buffers with a high pH, and the like and analyzed, for example, by Surface Plasmon Resonance (SPR).

### B. Antigen Binding Domains to Tumor Target Antigens

In addition to the described CD3 and half-life extension domains, the polypeptide constructs described herein also comprise at least one or at least two, or more domains that bind to one or more target antigens or one or more regions on a single target antigen. It is contemplated herein that a polypeptide construct of the invention is cleaved, for example, in a disease-specific microenvironment or in the blood of a subject at the protease cleavage domain and that each target antigen binding domain will bind to a target antigen on a target cell, thereby activating the CD3 binding domain to bind a T cell. In general, the TTA binding domains can bind to their targets before protease cleavage, so they can “wait” on the target cell to be activated as T-cell engagers. At least one target antigen is involved in and/or associated with a disease, disorder or condition. Exemplary target antigens include those associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In some embodiments, a target antigen is a tumor antigen expressed on a tumor cell. Alternatively in some embodiments, a target antigen is associated with a pathogen such as a virus or bacterium. At least one target antigen may also be directed against healthy tissue.

In some embodiments, a target antigen is a cell surface molecule such as a protein, lipid or polysaccharide. In some embodiments, a target antigen is a on a tumor cell, virally infected cell; bacterially infected cell, damaged red blood cell, arterial plaque cell, or fibrotic tissue cell. It is contemplated herein that upon binding more than one target antigen, two inactive CD3 binding domains are co-localized and form an active CD3 binding domain on the surface of the target cell. In some embodiments, the antigen binding protein comprises more than one target antigen binding domain to activate an inactive CD3 binding domain in the antigen binding protein. In some embodiments the antigen binding protein comprises more than one target antigen binding domain to enhance the strength of binding to the target cell. In some embodiments the antigen binding protein comprises more than one target antigen binding domain to enhance the strength of binding to the target cell. In some embodiments, more than one antigen binding domain comprise the same antigen binding domain. In some embodiments, more than one antigen binding domain comprise different antigen binding domains. For example, two different antigen binding domains known to be dually expressed in a diseased cell or tissue, for example a tumor or cancer cell, can enhance binding or selectivity of an antigen binding protein for a target.

Polypeptide constructs contemplated herein include at least one antigen binding domain, wherein the antigen binding domain binds to at least one target antigen. Target antigens, in some cases, are expressed on the surface of a diseased cell or tissue, for example a tumor or a cancer cell. Target antigens include but are not limited to epithelial cell adhesion molecule (EpCAM), epidermal growth factor receptor (EGFR), human epidermal growth factor receptor 2 (HER-2), human epidermal growth factor receptor 3 (HER-3), c-Met, folate receptor 1 (FOLR1), B7H3 (CD276), LY6/PLAUR domain containing 3 (LYPD3), carcinoembryonic antigen (CEA), carbonic anhydrase 9 (CA9 or CAIX), and tumor-associated calcium signal transducer 2 (Trop2). In some embodiments; one, two or more antigen binding domains of the constructs provided herein bind to EGFR, EpCAM, B7H3, FOLR1, Trop2, and CA9.

Polypeptide constructs disclosed herein, also include proteins comprising two antigen binding domains that bind to two different target antigens known to be expressed on a diseased cell or tissue. Exemplary pairs of antigen binding domains include, but are not limited to EGFR/EpCAM, EGFR/FOLR1, EGFR/B7H3, EpCAM/FOLR1, EpCAM/B7H3, EpCAM/BCMA, FOLR1/B7H3, B7H3/EpCAM; Trop2/EGFR, Trop2/EPCAM, Trop2/B7H3, Trop2/FOLR1, Trop2/CA9, CA9/EGFR, CA9/EPCAM, CA9/B7H3, CA9/FOLR1, and the like.

The design of the polypeptide constructs described herein allows the binding domain to one or more target antigens to be flexible in that the binding domain to a target antigen can be any type of binding domain; including but not limited to, domains from a monoclonal antibody, a polyclonal antibody, a recombinant antibody, a human antibody, a humanized antibody. In some embodiments, the binding domain to a target antigen is a single chain variable fragment (scFv), single-domain antibody such as a heavy chain variable domain (VH), a light chain variable domain (VL) and a variable domain (VHH) of camelid derived nanobody. In other embodiments, the binding domain to a target antigen is a non-Ig binding domain, i.e., antibody mimetic, such as anticalins, affilins, affibody molecules, affimers, affitins, alphabodies, avimers, DARPins, fynomers, kunitz domain peptides, and monobodies. In further embodiments, the binding domain to one or more target antigens is a ligand, a receptor domain, a lectin, or peptide that binds to or associates with one or more target antigens.

In some embodiments, the target cell antigen binding domains independently comprise a scFv, a VH domain, a VL domain, a non-Ig domain, or a ligand that specifically binds to the target antigen. In some embodiments, the target antigen binding domains specifically bind to a cell surface molecule. In some embodiments, the target antigen binding domains specifically bind to a tumor antigen. In some embodiments, the target antigen binding domains specifically and independently bind to an antigen selected from at least one of EGFR, HER-2, HER-3, cMet, LyPD3, CA9, CEA FOLR1, B7H3, EpCAM, and Trop2. In some embodiments, the target antigen binding domains specifically and independently bind to two different antigens, wherein at least one of the antigens is selected from one of EGFR, HER-2, HER-3, cMet, LyPD3, CEA FOLR1, B7H3, EpCAM, Trop2, and CA9. In some embodiments, the target antigen binding domains specifically and independently bind to an antigen selected from at least one of EGFR, FOLR1, B7H3, EpCAM, Trop2, and CA9. In some embodiments, the target antigen binding domains specifically and independently bind to two different antigens, wherein at least one of the antigens is selected from one of EGFR, FOLR1, B7H3, EpCAM, Trop2, and CA9.

In many embodiments, the antigen binding domain (ABD) to the target tumor antigen (TTA) is a single domain antigen binding domain (sdABD-TTA), based on cameliad single domain antibodies (sdABDs). sdABD-TTAs have framework regions just as traditional antibodies, as well as three CDRs, but do not have any heavy chain constant domains. These sdABD-TTAs are generally preferable over scFvs that bind TTAs, since the intramolecular folding that results in the formation of inactive Fvs that do not bind to CD3 is less complicated with fewer VH and VL domains. These sdABD-TTAs can be labeled by the target to which they bind, e.g., sdABD-EGFR is an sdABD that binds to human EGFR, etc.

In some embodiments, the antigen binding domain binds EGFR and has the amino acid sequence set forth in SEQ ID NO:50 or shown as anti-EGFR1 in FIG. 1A. In some embodiments, the antigen binding domain binds EGFR and has a humanized version of the amino acid sequence set forth in SEQ ID NO:50 or shown as anti-EGFR1 in FIG. 1A. In other embodiments, the antigen binding domain binds EGFR and has the CDRs and/or variable domains of the sequence set forth in SEQ ID NO:50 or shown as anti-EGFR1 in FIG. 1A.

In other embodiments, the antigen binding domain binds EGFR and has the amino acid sequence set forth in SEQ ID NO:54 or shown as anti-EGFR2 in FIG. 1A. In other embodiments, the antigen binding domain binds EGFR and has a humanized version of the amino acid sequence set forth in SEQ ID NO:54 or shown as anti-EGFR2 in FIG. 1A. In some embodiments, the antigen binding domain binds EGFR and has the CDRs and/or variable domains of the sequence set forth in SEQ ID NO:54 or shown as anti-EGFR2 in FIG. 1A.

In some embodiments, the antigen binding domain binds EGFR and has the amino acid sequence set forth in SEQ ID NO:58 or shown as anti-EGFR1 in FIG. 1A. In other embodiments, the antigen binding domain binds EGFR and has a humanized version of the amino acid sequence set forth in SEQ ID NO:58 or shown as humanized anti-EGFR1 in FIG. 1A. In other embodiments, the antigen binding domain binds EGFR and has the CDRs and/or variable domains of the sequence set forth in SEQ ID NO:58 or shown as anti-EGFR1 in FIG. 1A.

In certain embodiments, the antigen binding domain binds EGFR and has the amino acid sequence set forth in SEQ ID NO:62 or shown as anti-EGFR2a sdAb in FIG. 1A. In other embodiments, the antigen binding domain binds EGFR and has a humanized version of the amino acid sequence set forth in SEQ ID NO:62 or shown as humanized anti-EGFR2a in FIG. 1A. In various embodiments, the antigen binding domain binds EGFR and has the CDRs and/or variable domains of the sequence set forth in SEQ ID NO:62 or shown as anti-EGFR2a in FIG. 1A.

In certain embodiments, the antigen binding domain binds EGFR and has the amino acid sequence set forth in SEQ ID NO:66 or shown as anti-EGFR2d in FIG. 1A. In other embodiments, the antigen binding domain binds EGFR and has a humanized version of the amino acid sequence set forth in SEQ ID NO:66 or shown as humanized anti-EGFR2d in FIG. 1A. In various embodiments, the antigen binding domain binds EGFR and has the CDRs and/or variable domains of the sequence set forth in SEQ ID NO:66 or shown as anti-EGFR2d in FIG. 1A.

In some embodiments, the antigen binding domain binds FOLR1 and has the amino acid sequence set forth in SEQ ID NO:70 or shown as anti-FOLR1 h77-2 in FIG. 1B. In other embodiments, the antigen binding domain binds FOLR1 and has a humanized version of the amino acid sequence set forth in SEQ ID NO:70 or shown as anti-FOLR1 h77-2 in FIG. 1B. In some embodiments, the antigen binding domain binds FOLR1 and has the CDRs and/or variable domains of the sequence set forth in SEQ ID NO:70 or shown as anti-FOLR1 h77-2 in FIG. 1B.

In some embodiments, the antigen binding domain binds FOLR1 and has the amino acid sequence set forth in SEQ ID NO:74 or shown as anti-FOLR1 h59.3 in FIG. 1B. In other embodiments, the antigen binding domain binds FOLR1 and has a humanized version of the amino acid sequence set forth in SEQ ID NO:74 or shown as anti-FOLR1 h59.3 in FIG. 1B. In some embodiments, the antigen binding domain binds FOLR1 and has the CDRs and/or variable domains of the sequence set forth in SEQ ID NO:74 or shown as anti-FOLR1 h59.3 in FIG. 1B.

In some embodiments, the antigen binding domain binds FOLR1 and has the amino acid sequence set forth in SEQ ID NO:78 or shown as anti-FOLR1 h22-4 in FIG. 1B. In other embodiments, the antigen binding domain binds FOLR1 and has a humanized version of the amino acid sequence set forth in SEQ ID NO:78 or shown as anti-FOLR1 h22-4 in FIG. 1B. In some embodiments, the antigen binding domain binds FOLR1 and has the CDRs and/or variable domains of the sequence set forth in SEQ ID NO:78 or shown as anti-FOLR1 h22-4 in FIG. 1B.

In some embodiments, the antigen binding domain binds B7H3 and has the amino acid sequence set forth in SEQ ID NO:82 or shown as anti-B7H3 hF7 in FIG. 1B. In other embodiments, the antigen binding domain binds B7H3 and has a humanized version of the amino acid sequence set forth in SEQ ID NO:82 or shown as anti-B7H3 hF7 in FIG. 1B. In some embodiments, the antigen binding domain binds B7H3 and has the CDRs and/or variable domains of the sequence set forth in SEQ ID NO:82 or shown as anti-B7H3 hF7 in FIG. 1B.

In some embodiments, the antigen binding domain binds B7H3 and has the amino acid sequence set forth in SEQ ID NO:86 or shown as anti-B7H3 hF12 in FIG. 1C. In other embodiments, the antigen binding domain binds B7H3 and has a humanized version of the amino acid sequence set forth in SEQ ID NO:86 or shown as anti-B7H3 hF12 in FIG. 1C. In some embodiments, the antigen binding domain binds B7H3 and has the CDRs and/or variable domains of the sequence set forth in SEQ ID NO:86 or shown as anti-B7H3 hF12 in FIG. 1C. In some embodiments, the ABD that binds B7H3 is modified to remove an N-linked glycosylation site. In some embodiments, the Fc fusion protein of the present invention comprises an anti-B7H3 sdAb variant of the parental anti-B7H3 hF12 sdAB (SEQ ID NO:86) comprising one or more amino acid modifications selected from the group N57Q, N57E, N57D, S59A, and S59Y.

In some embodiments, the antigen binding domain binds B7H3 and has the amino acid sequence set forth in SEQ ID NO:90 or shown as anti-B7H3 hF12 (N57Q) in FIG. 1C. In other embodiments, the antigen binding domain binds B7H3 and has a humanized version of the amino acid sequence set forth in SEQ ID NO:90 or shown as anti-B7H3 hF12 (N57Q) in FIG. 1C. In some embodiments, the antigen binding domain binds B7H3 and has the CDRs and/or variable domains of the sequence set forth in SEQ ID NO:90 or shown as anti-B7H3 hF12 (N57Q) in FIG. 1C

In some embodiments, the antigen binding domain binds B7H3 and has the amino acid sequence set forth in SEQ ID NO:94 or shown as anti-B7H3 hF12 (N57E) in FIG. 1C. In other embodiments, the antigen binding domain binds B7H3 and has a humanized version of the amino acid sequence set forth in SEQ ID NO:94 or shown as anti-B7H3 hF12 (N57E) in FIG. 1C. In some embodiments, the antigen binding domain binds B7H3 and has the CDRs and/or variable domains of the sequence set forth in SEQ ID NO:94 or shown as anti-B7H3 hF12 (N57E) in FIG. 1C.

In some embodiments, the antigen binding domain binds B7H3 and has the amino acid sequence set forth in SEQ ID NO:98 or shown as anti-B7H3 hF12 (N57D) in FIG. 1C. In other embodiments, the antigen binding domain binds B7H3 and has a humanized version of the amino acid sequence set forth in SEQ ID NO:98 or shown as anti-B7H3 hF12 (N57D) in FIG. 1C. In some embodiments, the antigen binding domain binds B7H3 and has the CDRs and/or variable domains of the sequence set forth in SEQ ID NO:98 or shown as anti-B7H3 hF12 (N57D) in FIG. 1C.

In some embodiments, the antigen binding domain binds B7H3 and has the amino acid sequence set forth in SEQ ID NO:102 or shown as anti-B7H3 hF12 (S59A) in FIG. 1D. In other embodiments, the antigen binding domain binds B7H3 and has a humanized version of the amino acid sequence set forth in SEQ ID NO:102 or shown as anti-B7H3 hF12 (S59A) in FIG. 1D. In some embodiments, the antigen binding domain binds B7H3 and has the CDRs and/or variable domains of the sequence set forth in SEQ ID NO:102 or shown as anti-B7H3 hF12 (S59A) in FIG. 1D.

In some embodiments, the antigen binding domain binds B7H3 and has the amino acid sequence set forth in SEQ ID NO:106 or shown as anti-B7H3 hF12 (S59Y) in FIG. 1D. In other embodiments, the antigen binding domain binds B7H3 and has a humanized version of the amino acid sequence set forth in SEQ ID NO:106 or shown as anti-B7H3 hF12 (S59Y) in FIG. 1D. In some embodiments, the antigen binding domain binds B7H3 and has the CDRs and/or variable domains of the sequence set forth in SEQ ID NO:106 or shown as anti-B7H3 hF12 (S59Y) in FIG. 1D.

In some embodiments, the antigen binding domain binds EpCAM and has the amino acid sequence set forth in SEQ ID NO:110 or shown as anti-EpCAM h13 in FIG. 1D. In other embodiments, the antigen binding domain binds EpCAM and has a humanized version of the amino acid sequence set forth in SEQ ID NO:110 or shown as anti-EpCAM h13 in FIG. 1D. In some embodiments, the antigen binding domain binds EpCAM and has the CDRs and/or variable domains of the sequence set forth in SEQ ID NO:110 or shown as anti-EpCAM h13 in FIG. 1D. In some embodiments, the Fc fusion protein of the present invention comprises an anti-EpCAM sdAb that binds uncleaved EpCAM. In some embodiments, the Fc fusion protein of the present invention comprises an anti-EpCAM sdAb that binds cleaved EpCAM. In some embodiments, the anti-EpCAM sdAb binds uncleaved and cleaved EpCAM.

In some embodiments, the antigen binding domain binds EpCAM and has the amino acid sequence set forth in SEQ ID NO:114 or shown as anti-EpCAM h23 in FIG. 1D. In other embodiments, the antigen binding domain binds EpCAM and has a humanized version of the amino acid sequence set forth in SEQ ID NO:114 or shown as anti-EpCAM h23 in FIG. 1D. In some embodiments, the antigen binding domain binds EpCAM and has the CDRs and/or variable domains of the sequence set forth in SEQ ID NO:114 or shown as anti-EpCAM h23 in FIG. 1D. In some embodiments, the Fc fusion protein of the present invention comprises an anti-EpCAM sdAb that binds uncleaved EpCAM. In some embodiments, the Fc fusion protein of the present invention comprises an anti-EpCAM sdAb that binds cleaved EpCAM. In some embodiments, the anti-EpCAM sdAb binds uncleaved and cleaved EpCAM.

In some embodiments, the antigen binding domain binds EpCAM and has the amino acid sequence set forth in SEQ ID NO:118 or shown as anti-EpCAM hVIB665 in FIG. 1E. In other embodiments, the antigen binding domain binds EpCAM and has a humanized version of the amino acid sequence set forth in SEQ ID NO:118 or shown as anti-EpCAM hVIB665 in FIG. 1E. In some embodiments, the antigen binding domain binds EpCAM and has the CDRs and/or variable domains of the sequence set forth in SEQ ID NO:118 or shown as anti-EpCAM hVIB665 in FIG. 1E. In some embodiments, the Fc fusion protein of the present invention comprises an anti-EpCAM sdAb that binds uncleaved EpCAM. In some embodiments, the Fc fusion protein of the present invention comprises an anti-EpCAM sdAb that binds cleaved EpCAM. In some embodiments, the anti-EpCAM sdAb binds uncleaved and cleaved EpCAM.

In some embodiments, the antigen binding domain binds EpCAM and has the amino acid sequence set forth in SEQ ID NO:122 or shown as anti-EpCAM hVIB666 in FIG. 1E. In other embodiments, the antigen binding domain binds EpCAM and has a humanized version of the amino acid sequence set forth in SEQ ID NO:122 or shown as anti-EpCAM hVIB666 in FIG. 1E. In some embodiments, the antigen binding domain binds EpCAM and has the CDRs and/or variable domains of the sequence set forth in SEQ ID NO:122 or shown as anti-EpCAM hVIB666 in FIG. 1E. In some embodiments, the Fc fusion protein of the present invention comprises an anti-EpCAM sdAb that binds uncleaved EpCAM. In some embodiments, the Fc fusion protein of the present invention comprises an anti-EpCAM sdAb that binds cleaved EpCAM. In some embodiments, the anti-EpCAM sdAb binds uncleaved and cleaved EpCAM.

In some embodiments, the antigen binding domain binds Trop2 and has the amino acid sequence set forth in SEQ ID NO:126 or shown as anti-Trop2 hVIB557 in FIG. 1E. In other embodiments, the antigen binding domain binds Trop2 and has a humanized version of the amino acid sequence set forth in SEQ ID NO:126 or shown as anti-Trop2 hVIB557 in FIG. 1E. In some embodiments, the antigen binding domain binds Trop2 and has the CDRs and/or variable domains of the sequence set forth in SEQ ID NO:126 or shown as anti-Trop2 hVIB557 in FIG. 1E.

In some embodiments, the antigen binding domain binds Trop2 and has the amino acid sequence set forth in SEQ ID NO:130 or shown as anti-Trop2 hVIB565 in FIG. 1E. In other embodiments, the antigen binding domain binds Trop2 and has a humanized version of the amino acid sequence set forth in SEQ ID NO:130 or shown as anti-Trop2 hVIB565 in FIG. 1E. In some embodiments, the antigen binding domain binds Trop2 and has the CDRs and/or variable domains of the sequence set forth in SEQ ID NO:130 or shown as anti-Trop2 hVIB565 in FIG. 1E.

In some embodiments, the antigen binding domain binds Trop2 and has the amino acid sequence set forth in SEQ ID NO:134 or shown as anti-Trop2 hVIB575 in FIG. 1F. In other embodiments, the antigen binding domain binds Trop2 and has a humanized version of the amino acid sequence set forth in SEQ ID NO:134 or shown as anti-Trop2 hVIB575 in FIG. 1F. In some embodiments, the antigen binding domain binds Trop2 and has the CDRs and/or variable domains of the sequence set forth in SEQ ID NO:134 or shown as anti-Trop2 hVIB575 in FIG. 1F.

In some embodiments, the antigen binding domain binds Trop2 and has the amino acid sequence set forth in SEQ ID NO:138 or shown as anti-Trop2 hVIB578 in FIG. 1F. In other embodiments, the antigen binding domain binds Trop2 and has a humanized version of the amino acid sequence set forth in SEQ ID NO:138 or shown as anti-Trop2 hVIB578 in FIG. 1F. In some embodiments, the antigen binding domain binds Trop2 and has the CDRs and/or variable domains of the sequence set forth in SEQ ID NO:138 or shown as anti-Trop2 hVIB578 in FIG. 1F.

In some embodiments, the antigen binding domain binds Trop2 and has the amino acid sequence set forth in SEQ ID NO:142 or shown as anti-Trop2 hVIB609 in FIG. 1F. In other embodiments, the antigen binding domain binds Trop2 and has a humanized version of the amino acid sequence set forth in SEQ ID NO:142 or shown as anti-Trop2 hVIB609 in FIG. 1F. In some embodiments, the antigen binding domain binds Trop2 and has the CDRs and/or variable domains of the sequence set forth in SEQ ID NO:142 or shown as anti-Trop2 hVIB609 in FIG. 1F.

In some embodiments, the antigen binding domain binds Trop2 and has the amino acid sequence set forth in SEQ ID NO:146 or shown as anti-Trop2 hVIB619 in FIG. 1F. In other embodiments, the antigen binding domain binds Trop2 and has a humanized version of the amino acid sequence set forth in SEQ ID NO:146 or shown as anti-Trop2 hVIB619 in FIG. 1F. In some embodiments, the antigen binding domain binds Trop2 and has the CDRs and/or variable domains of the sequence set forth in SEQ ID NO:146 or shown as anti-Trop2 hVIB619 in FIG. 1F.

In some embodiments, the antigen binding domain binds CA9 and has the amino acid sequence set forth in SEQ ID NO:150 or shown as anti-CA9 hVIB456 in FIG. 1F. In other embodiments, the antigen binding domain binds CA9 and has a humanized version of the amino acid sequence set forth in SEQ ID NO:150 or shown as anti-CA9 hVIB456 in FIG. 1F. In some embodiments, the antigen binding domain binds CA9 and has the CDRs and/or variable domains of the sequence set forth in SEQ ID NO:150 or shown as anti-CA9 hVIB456 in FIG. 1F.

In some embodiments, the antigen binding domain binds CA9 and has the amino acid sequence set forth in SEQ ID NO:154 or shown as anti-CA9 hVIB476 in FIG. 1G. In other embodiments, the antigen binding domain binds CA9 and has a humanized version of the amino acid sequence set forth in SEQ ID NO:154 or shown as anti-CA9 hVIB476 in FIG. 1G. In some embodiments, the antigen binding domain binds CA9 and has the CDRs and/or variable domains of the sequence set forth in SEQ ID NO:154 or shown as anti-CA9 hVIB476 in FIG. 1G.

In some embodiments, the antigen binding domain binds CA9 and has the amino acid sequence set forth in SEQ ID NO:158 or shown as anti-CA9 hVIB407 in FIG. 1G. In other embodiments, the antigen binding domain binds CA9 and has a humanized version of the amino acid sequence set forth in SEQ ID NO:158 or shown as anti-CA9 hVIB407 in FIG. 1G. In some embodiments, the antigen binding domain binds CA9 and has the CDRs and/or variable domains of the sequence set forth in SEQ ID NO:158 or shown as anti-CA9 hVIB407 in FIG. 1G.

In some embodiments, the antigen binding domain binds CA9 and has the amino acid sequence set forth in SEQ ID NO:162 or shown as anti-CA9 hVIB445 in FIG. 1G. In other embodiments, the antigen binding domain binds CA9 and has a humanized version of the amino acid sequence set forth in SEQ ID NO:162 or shown as anti-CA9 hVIB445 in FIG. 1G. In some embodiments, the antigen binding domain binds CA9 and has the CDRs and/or variable domains of the sequence set forth in SEQ ID NO:162 or shown as anti-CA9 hVIB445 in FIG. 1G.

In some embodiments, the protein prior to cleavage of the protease cleavage domain is less than about 100 kDa. In some embodiments, the protein after cleavage of the protease cleavage domain is about 25 to about 75 kDa. In some embodiments, the protein prior to protease cleavage has a size that is above the renal threshold for first-pass clearance. In some embodiments, the protein prior to protease cleavage has an elimination half-time of at least about 50 hours. In some embodiments, the protein prior to protease cleavage has an elimination half-time of at least about 100 hours. In some embodiments, the protein has increased tissue penetration as compared to an IgG to the same target antigen. In some embodiments, the protein has increased tissue distribution as compared to an IgG to the same target antigen.

### C. Half-Life Extension

The proteins of the invention optionally include half-life extension domains. Such domains are contemplated to include but are not limited to HSA binding domains, Fc regions, small molecules, and other half-life extension domains known in the art.

1. Fc Regions

The proteins of the present invention include Fc domain-fusion proteins that combine the Fc region of an antibody with additional components as outlined herein, including ABDs to TTAs and Fv domains, generally pseudo domains as outlined herein.

The knob-in-hole format of the heterodimeric Fc proteins describe herein refer to amino acid substitution(s) that create “steric influences” to favor heterodimeric formation over homodimeric formations. In some cases, the knob-in-hole format can be combined with disulfide bonds or pairs of charged amino acid substitutions to further favor heterodimeric formation.

In some embodiments, the heterodimeric Fc proteins comprise an Fc arm comprising either a knob or a hole in the Fc region. In other words, the first monomeric Fc arm comprises a knob and the second monomeric Fc arm comprises a hole. In embodiments of “construct 6” or “construct 7”, the monomeric Fc arm containing an active Fv domain (e.g., anti-CD3 variable heavy chain and variable light chain) includes a CH3-knob, and the monomeric Fc arm containing a pseudo Fv domain (e.g., inactive variable heavy chain and inactive variable light chain) comprises a CH3-hole, although this can be reversed, as well. In other embodiments, the monomeric Fc arm containing an active Fv domain includes a CH3-hole, and the monomeric Fc arm containing a pseudo Fv domain comprises a CH3-knob.

Amino acid residues for the formation of a knob are generally naturally occurring amino acid residues and are selected from arginine (R), phenylalanine (F), tyrosine (Y) and tryptophan (W). In some preferred embodiments, the amino acid residues are tryptophan and tyrosine. In one embodiment, the original residue for the formation of the knob has a small side chain volume, such as alanine, asparagine, aspartic acid, glycine, serine, threonine or valine. Exemplary amino acid substitutions in the CH3 domain for forming the knob include without limitation the T366W, T366Y or F405W substitution.

Amino acid residues for the formation of a hole are usually naturally occurring amino acid residues and are selected from alanine (A), serine (S), threonine (T) and valine (V). In some preferred embodiments, the original residue for the formation of the hole has a large side chain volume, such as tyrosine, arginine, phenylalanine or tryptophan. Exemplary amino acid substitutions in the CH3 domain for generating the hole include without limitation the T366S, L368A, F405A, Y407A, Y407T and Y407V substitutions. In certain embodiments, the knob comprises T366W substitution, and the hole comprises the T366S/L368A/Y407V substitutions.

In general, preferred Fc domains for use herein are human IgG domains, and generally either IgG1 or IgG4. In some instances, for example when effector function is undesirable, IgG4 is used, and in some cases contains a S228P variant in the hinge domain, as this prevents arm exchange.

It is understood that other modifications to the Fc region known in the art that facilitate heterodimerization are also contemplated and encompassed by the instant application.

In some embodiments, the Fc region of the formats described herein include a tag such as, but not limited to, a histidine tag (e.g., (His)6)), a streptavidin tag (e.g., strep-tag or Strep-tag II), or a maltose-binding protein (MBP) tag at the C-terminus of the Fc.

Additionally, the Fc domains may contain additional amino acid modifications to alter effector function or half life, as is known in the art.

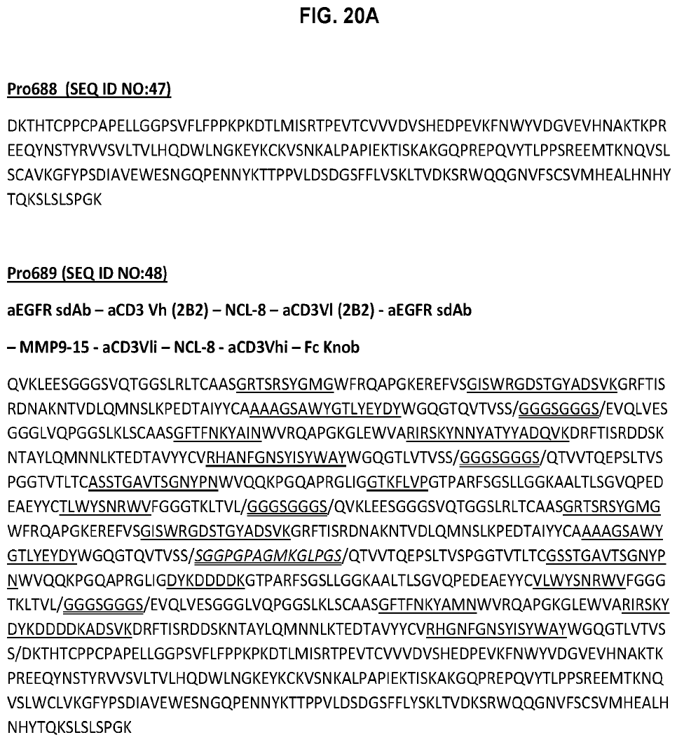

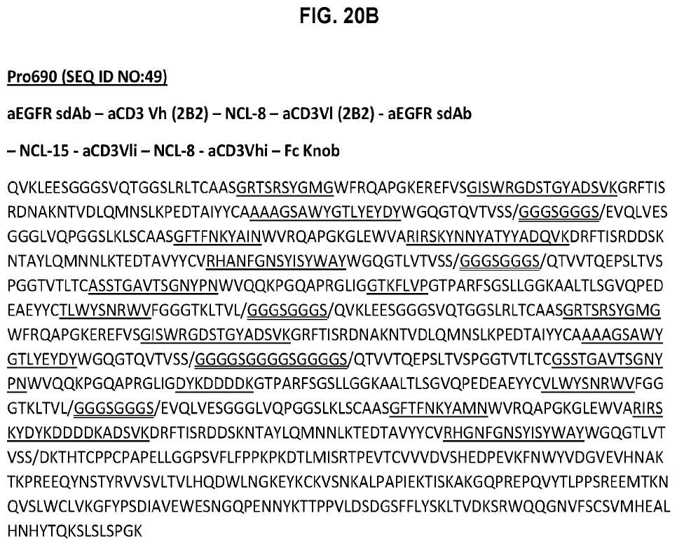

In some embodiments, the Fc region of the formats described herein is depicted in FIGS. 17A-18C and FIGS. 20A-20B. In some embodiments, the amino acid sequence of the Fc region is provided in SEQ ID NO:36 (Pro556), SEQ ID NO:37 (Pro557), SEQ ID NO:38 (Pro587), SEQ ID NO:39 (Pro588), SEQ ID NO:40 (Pro589), SEQ ID NO:41 (Pro574), SEQ ID NO:42 (Pro575), SEQ ID NO:43 (Pro576), SEQ ID NO:44 (Pro584), SEQ ID NO:45 (Pro585), SEQ ID NO:46 (Pro586), SEQ ID NO:47 (Pro688), SEQ ID NO:48 (Pro689), and SEQ ID NO:49 (Pro690).

2. Human Serum Albumin Binding Domain

Human serum albumin (HSA) (molecular mass ˜67 kDa) is the most abundant protein in plasma, present at about 50 mg/ml (600 μM), and has a half-life of around 20 days in humans. HSA serves to maintain plasma pH, contributes to colloidal blood pressure, functions as carrier of many metabolites and fatty acids, and serves as a major drug transport protein in plasma.

Noncovalent association with albumin extends the elimination half-time of short lived proteins. For example, a recombinant fusion of an albumin binding domain to a Fab fragment resulted in a reduced in vivo clearance of 25- and 58-fold and a half-life extension of 26- and 37-fold when administered intravenously to mice and rabbits respectively as compared to the administration of the Fab fragment alone. In another example, when insulin is acylated with fatty acids to promote association with albumin, a protracted effect was observed when injected subcutaneously in rabbits or pigs. Together, these studies demonstrate a linkage between albumin binding and prolonged action.

In one aspect, the antigen-binding proteins described herein comprise a half-life extension domain, for example a domain which specifically binds to HSA. In some embodiments, the HSA binding domain of an antigen binding protein can be any domain that binds to HSA including but not limited to domains from a monoclonal antibody, a polyclonal antibody, a recombinant antibody, a human antibody, a humanized antibody. In some embodiments, the HSA binding domain is a single chain variable fragments (scFv), single-domain antigen binding domain (sdABD) such as a heavy chain variable domain (VH), a light chain variable domain (VL) and a variable domain (VHH) of camelid derived nanobody, peptide, ligand or small molecule specific for HSA. In certain embodiments, the HSA binding domain is from a single-domain antibody (sdABD) and comprises a single domain antigen binding domain (sdABD); that is, a sdABD is a single variable domain (VHH) that contains three CDRs, rather than the standard six CDRs in an Fv of traditional antibodies. In other embodiments, the HSA binding domain is a peptide. In further embodiments, the HSA binding domain is a small molecule. It is contemplated that the HSA binding domain of an antigen binding protein is fairly small and no more than 25 kD, no more than 20 kD, no more than 15 kD, or no more than 10 kD in some embodiments. In certain instances, the HSA binding domain is 5 kD or less if it is a peptide or small molecule.

The half-life extension domain of an antigen binding protein provides for altered pharmacodynamics and pharmacokinetics of the antigen binding protein itself. As above, the half-life extension domain extends the elimination half-time. The half-life extension domain also alters pharmacodynamic properties including alteration of tissue distribution, penetration, and diffusion of the antigen-binding protein. In some embodiments, the half-life extension domain provides for improved tissue (including tumor) targeting, tissue penetration, tissue distribution, diffusion within the tissue, and enhanced efficacy as compared with a protein without a half-life extension binding domain. In one embodiment, therapeutic methods effectively and efficiently utilize a reduced amount of the antigen-binding protein, resulting in reduced side effects, such as reduced non-tumor cell cytotoxicity.

Further, characteristics of the half-life extension domain, for example a HSA binding domain, include the binding affinity of the HSA binding domain for HSA. Affinity of the HSA binding domain can be selected so as to target a specific elimination half-time in a particular polypeptide construct. Thus, in some embodiments, the HSA binding domain has a high binding affinity. In other embodiments, the HSA binding domain has a medium binding affinity. In yet other embodiments, the HSA binding domain has a low or marginal binding affinity. Exemplary binding affinities include KD concentrations at 10 nM or less (high), between 10 nM and 100 nM (medium), and greater than 100 nM (low). As above, binding affinities to HSA are determined by known methods such as Surface Plasmon Resonance (SPR).

### D. Protease Cleavage Sites

The polypeptide (e.g., protein) compositions of the invention, and particularly the prodrug constructs, include one or more protease cleavage sites, generally resident in cleavable linkers, as outlined herein.

As described herein, the prodrug constructs of the invention include at least one protease cleavage site comprising an amino acid sequence that is cleaved by at least one protease. In some cases, the proteins described herein comprise 1, 2, 3, 4, 5, 6, 7, 8, 9, 10, 11, 12, 13, 14, 15, 16, 17, 18, 19, 20, or more protease cleavage sites that are cleaved by at least one protease. As is more fully discussed herein, when more than one protease cleavage site is used in a prodrug construction, they can be the same (e.g., multiple sites that are cleaved by a single protease) or different (two or more cleavage sites are cleaved by at least two different proteases). As will be appreciated by those in the art, constructs containing three or more protease cleavage sites can utilize one, two, three, etc.; e.g. some constructs can utilize three sites for two different proteases, etc.

The amino acid sequence of the protease cleavage site will depend on the protease that is targeted. As is known in the art, there are a number of human proteases that are found in the body and can be associated with disease states.

Proteases are known to be secreted by some diseased cells and tissues, for example tumor or cancer cells, creating a microenvironment that is rich in proteases or a protease-rich microenvironment. In some cases, the blood of a subject is rich in proteases. In some cases, cells surrounding the tumor secrete proteases into the tumor microenvironment. Cells surrounding the tumor secreting proteases include but are not limited to the tumor stromal cells, myofibroblasts, blood cells, mast cells, B cells, NK cells, regulatory T cells, macrophages, cytotoxic T lymphocytes, dendritic cells, mesenchymal stem cells, polymorphonuclear cells, and other cells. In some cases, proteases are present in the blood of a subject, for example proteases that target amino acid sequences found in microbial peptides. This feature allows for targeted therapeutics such as antigen-binding proteins to have additional specificity because T cells will not be bound by the antigen binding protein except in the protease rich microenvironment of the targeted cells or tissue.

Proteases are proteins that cleave proteins, in some cases, in a sequence-specific manner. Proteases include but are not limited to serine proteases, cysteine proteases, aspartate proteases, threonine proteases, glutamic acid proteases, metalloproteases, asparagine peptide lyases, serum proteases, Cathepsins (e.g., Cathepsin B, Cathepsin C, Cathepsin D, Cathepsin E, Cathepsin K, Cathepsin L, CathepsinS), kallikreins, hK1, hK10, hK15, KLK7, GranzymeB, plasmin, collagenase, Type IV collagenase, stromelysin, factor XA, chymotrypsin-like protease, trypsin-like protease, elastase-like protease, subtilisin-like protease, actinidain, bromelain, calpain, Caspases (e.g., Caspase-3), Mir1-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMPI, MMP2, MMP3, MMP8, MMP9, MMP13, MMP11, MMP14, meprin, urokinase plasminogen activator (uPA), enterokinase, prostate-specific antigen (PSA, hK3), interleukin-1β converting enzyme, thrombin, FAP (FAP-α), dipeptidyl peptidase, and dipeptidyl peptidase IV (DPPIV/CD26).

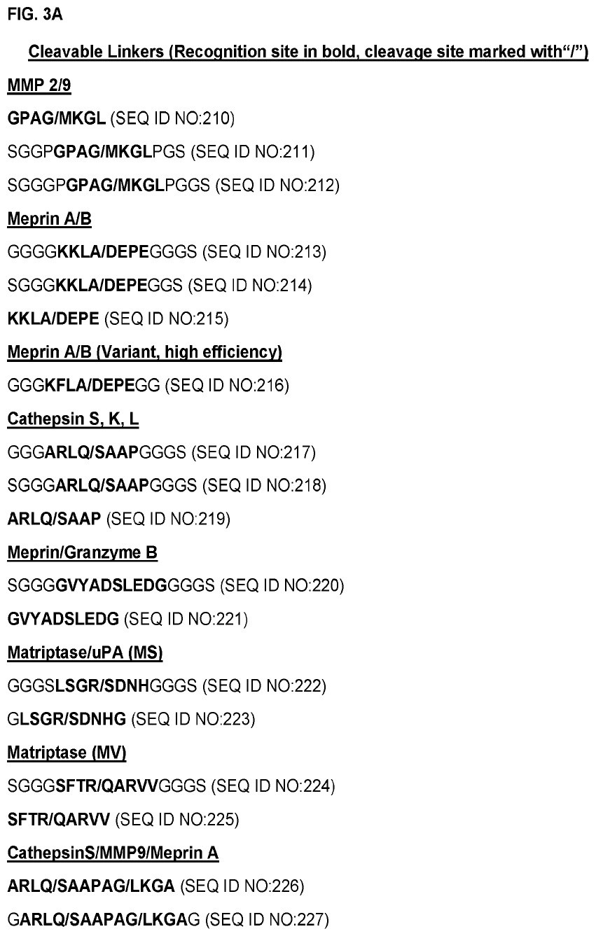

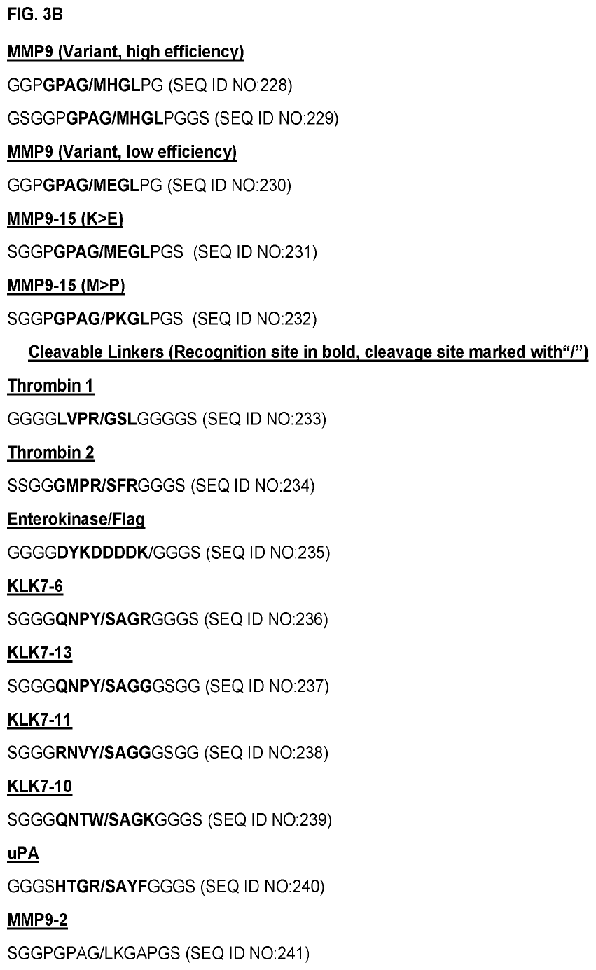

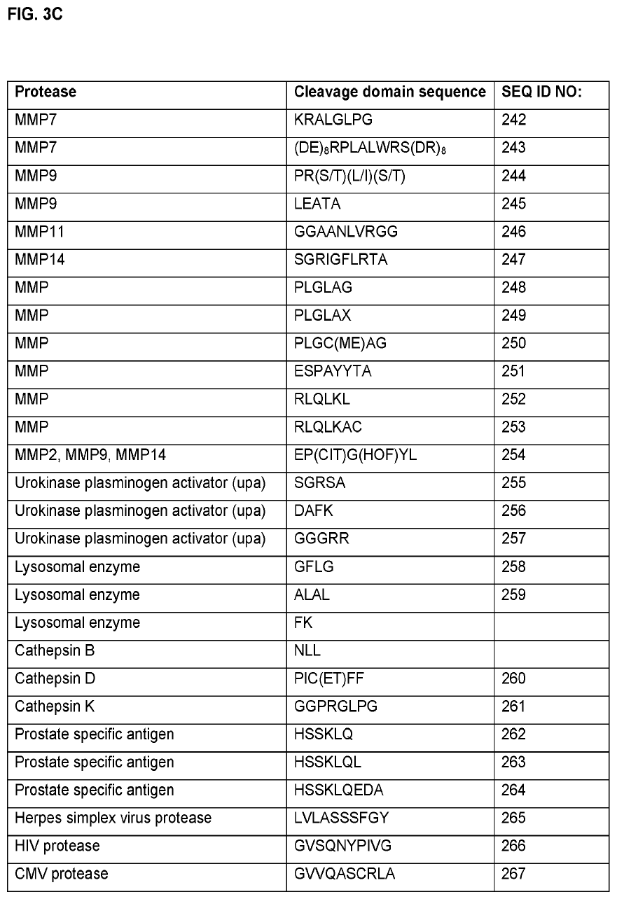

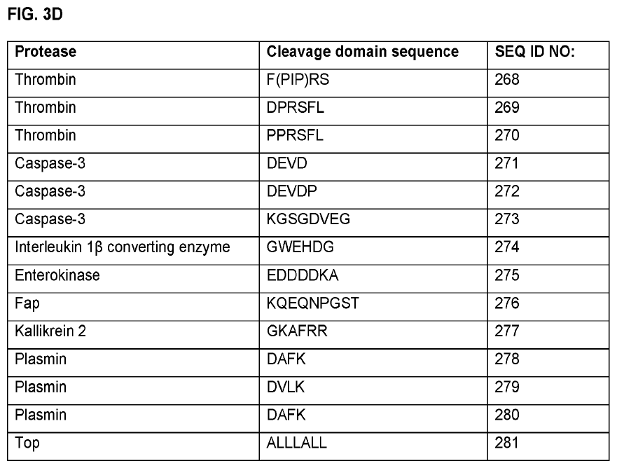

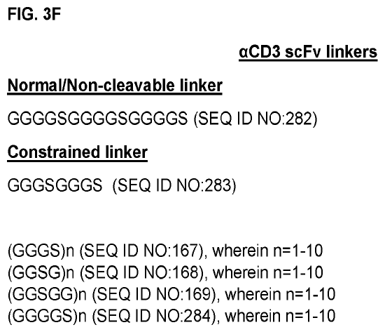

Some suitable proteases and protease cleavage sequences are set forth as SEQ ID NOS: 210-281, and are shown in FIGS. 3A-3D.

### E. Linkers

As is discussed herein, the different domains of the invention are generally linked together using amino acid linkers, which can confer functionality as well, including flexibility or inflexibility (e.g. steric constraint) as well as the ability to be cleaved using an in situ protease. These linkers can be classified in a number of ways.

The invention provides “domain linkers”, which are used to join two or more domains (e.g. a VH and a VL, a target tumor antigen binding domain (TTABD, sometimes also referred to herein as “αTTA” (for “anti-TTA”) to a VH or VL, a half-life extension domain to another component, etc. Domain linkers can be a non-cleavable linker (NCL), cleavable linker (“CL”), cleavable and constrained linker (CCL) and non-cleavable and constrained linker (NCCL), for example. In some embodiments, a constrained linker is a short polypeptide of less than 10 amino acids (e.g., 9, 8, 7, 6, 5, or 4 amino acids) that joins two domains as outlined herein in such a manner that the two domains cannot significantly interact with each other, and that is not significantly cleaved by human proteases under physiological conditions. In general, protease cleavage sites generally are at least 4+ amino acids in length to confer sufficient specificity, as is shown in FIGS. 3A-3D, 14A-14G, 16A-G, 17A-17C, 18A-18C, and 20A-20B.

1. Non-Cleavable Linkers

In one embodiment, the domain linker is a non-cleavable linker (NCL). In this embodiment, the linker is used to join domains to preserve the functionality of the domains, generally through longer, flexible domains that are not cleaved by in situ proteases in a patient. Examples of internal, non-cleavable linkers suitable for linking the domains in the polypeptides of the invention include but are not limited to (GS)n, (GGS)n, (GGGS)n (SEQ ID NO:167), (GGSG)n (SEQ ID NO:168), (GGSGG)n (SEQ ID NO:169), or (GGGGS)n (SEQ ID NO:284), wherein n is 1, 2, 3, 4, 5, 6, 7, 8, 9, or 10. Any non-cleavable domain linker recognized by one skilled in the art can be used in the homodimeric and heterodimeric Fc proteins described herein.

In some embodiments, the linkers do not contain a cleavage site and are also too short to allow the protein domains separated by the linker to intramolecularly self-assemble, and are “constrained non-cleavable linkers” or “CNCLs”. For example, in Pro219 and Pro217, an active VH and an active VL are separated by 8 amino acids (an “8mer”) that does not allow the VH and VL to intramolecularly self-assemble into an active antigen binding domain; instead, an intermolecular assembly with Pro218 happens instead, until cleavage by the tumor protease. In some embodiments, the linker is still flexible; for example, (GGGS)n where n=2. In other embodiments, although generally less preferred, more rigid linkers can be used, such as those that include proline or bulky amino acids.

In some embodiments, a linker include an scFV linker comprises any one of the sequences selected from the group consisting of SEQ ID NOS:167, 168, 169, 282, 283, and 284.

2. Cleavable Linkers

All of the prodrug constructs herein include at least one cleavable linker. Thus, in one embodiment, the domain linker is cleavable (CL), sometimes referred to herein as a “protease cleavage domain” (“PCD”). In this embodiment, the CL contains a protease cleavage site, as outlined herein and as depicted in FIGS. 3A, 3B, and 3C and the corresponding sequence listing. In some cases, the CL contains just the protease cleavage site. Optionally, depending on the length of the cleavage recognition site, there can be an extra few linking amino acids at either or both of the N- or C-terminal end of the CL; for example, there may be from 1, 2, 3, 4 or 5-8 amino acids on either or both of the N- and C-termini of the cleavage site.

### IV. Expression Methods

The invention provides nucleic acids encoding the two monomers of the heterodimeric proteins of the invention, and expression vectors and host cells. As will be appreciated by those in the art, either one or two expression vectors can be made. That is, a first nucleic acid encoding a first monomer and a second nucleic acid encoding a second monomer can be put into a single expression vector, or two expression vectors. The expression vector(s) are then put into host cells, which are grown such that the two monomers are expressed. In some cases, although this is generally not preferred, each monomer can be produced in a separate host cell and then the expression products combined to form the heterodimeric pro-drug proteins of the invention.

However, most embodiments rely on the use of co-expression of the two monomers. That is, provided herein are methods for producing proteins of the invention by co-expression in a cell (e.g., a host cell) and co-purification to obtain a first monomeric Fc polypeptide and a second monomeric Fc polypeptide. In some embodiments, the complementary pair of proteins (e.g., a first monomeric Fc polypeptide and a second monomeric Fc polypeptide) are produced at an about equimolar ratio (e.g., an about 1:1 ratio). In other embodiments, the complementary pair of proteins (e.g., a first monomeric Fc polypeptide and a second monomeric Fc polypeptide) are produced at a ratio that is not equimolar (e.g., not an about 1:1 ratio). In other words, the method described herein can be used to obtain a ratio of the first polypeptide to the second polypeptide such as, but not limited to, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1 10:1, 9:1, 8:1, 7:1, 6:1, 5:1, 4:1, 3:1, 2:1, 1:2, 1:3, 1:4, 1:5, 1:6, 1:7, 1:8, 1:9, 1:10, 1:15, 1:20, 1:25, 1:30, 1:35, 1:40, 1:45, 150, 1:55, 1:60, 1:65, 1:70, 1:75, 1:80, 1:85, 1:90, 195, 1:100, and the like.

A specific amount of the polynucleotide (or expression vector) encoding the polypeptide can be expressed in a cell to produce a desired amount of the polypeptide. In some embodiments, the amount of the first polynucleotide (or first expression vector) encoding the first monomeric Fc polypeptide and the amount of the polynucleotide (or expression vector) encoding the second monomeric Fc polypeptide that are introduced (e.g., transfected, electroporated, transduced, and the like) into the cell are the same. For instance, the first polynucleotide and the second polynucleotide can be introduced into a cell at a ratio of about 1:1. In other embodiments, the amount of the first polynucleotide (or first expression vector) encoding the first monomeric Fc polypeptide and the amount of the second polynucleotide (or expression vector) encoding the second monomeric Fc polypeptide that is introduced into the cell are different. For example, the first polynucleotide and the second polynucleotide can be introduced into a cell at a ratio such as, but not limited to, 50:1, 45:1, 40:1, 35:1, 30:1, 25:1, 20:1, 15:1 10:1, 9:1, 8:1, 7:1, 6:1, 5:1, 4:1, 3:1, 2:1, 1:2, 1:3, 1:4, 1:5, 1:6, 1:7, 1:8, 1:9, 1:10, 1:15, 1:20, 1:25, 1:30, 1:35, 1:40, 1:45, 1:50, and the like.

The expression vectors for the polypeptides can include one or more components (e.g., promoters, regulatory elements, enhancers, and the like) that enable production of the polypeptides at a desired ratio by the cell. In some cases, the first expression vector of the first monomeric Fc polypeptide comprises components that increase the expression level of the vector compared to the expression level of the second expression vector of the second polypeptide. In other cases, the second expression vector of the second monomeric Fc polypeptide comprises components that increase the expression level of the vector compared to the expression level of the first expression vector of the first monomeric Fc polypeptide. In certain cases, the first expression vector of the first monomeric Fc polypeptide comprises components such that the expression level of the vector is the same as the expression level of the second expression vector of the second monomeric Fc polypeptide.

In some cases, a nucleic acid described herein provides for production of bispecific conditionally effective proteins of the present disclosure, e.g., in a mammalian cell. A nucleotide sequence encoding the first and/or the second polypeptide of the present disclosure can be operably linked to a transcriptional control element, e.g., a promoter, and enhancer, etc.

Suitable promoter and enhancer elements are known in the art. For expression in a bacterial cell, suitable promoters include, but are not limited to, lad, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; EF-1a, mouse metallothionein-I promoter; and various art-known tissue specific promoters.

A nucleic acid or nucleotide sequence encoding a protein, e.g., a prodrug construct described herein can be present in an expression vector and/or a cloning vector. Where a protein, e.g., a prodrug construct comprises two separate polypeptides, nucleotide sequences encoding the two polypeptides can be cloned in the same or separate vectors. An expression vector can include a selectable marker, an origin of replication, and other features that provide for replication and/or maintenance of the vector. Suitable expression vectors include, e.g., plasmids, viral vectors, and the like.

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

The present disclosure provides a mammalian cell that is modified to produce a protein, e.g., a prodrug construct of the present disclosure. A polynucleotide described herein can be introduced into a mammalian cell using any method known to one skilled in the art such as, but not limited to, transfection, electroporation, viral infection, and the like.

Suitable mammalian cells include primary cells and immortalized cell lines. Suitable mammalian cell lines include human cell lines, non-human primate cell lines, rodent (e.g. mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HEK293 cells, expi293 cells, HLHepG2 cells, Hut-78, Jurkat, HL-60, NK cell lines (e.g., NKL, NK92, and YTS), and the like. Suitable host cells for cloning or expression of target protein-encoding vectors include prokaryotic or eukaryotic cells described herein.

For expression of polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli). After expression, the Fc fission protein may be isolated from the bacterial cell paste in a soluble fraction and can be further purified.

In addition to prokaryotes, eukaryotic microbes such as filamentous fungi or yeast are suitable cloning or expression hosts, including fungi and yeast strains whose glycosylation pathways have been “humanized,” resulting in the production of an antibody with a partially or fully human glycosylation pattern. See, e.g., Gerngross, Nat Biotech, 2004, 22:1409-1414, and Li et al., Nat Biotech, 2006, 24:210-215.

Plant cell cultures can also be utilized as hosts. See, e.g., U.S. Pat. Nos. 5,959,177, 6,040,498, 6,420,548, 7,125,978, and 6,417,429.

Suitable host cells for the expression of glycosylated proteins are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

In some embodiments, the host cell or stable host cell line is selected according to the amount of polypeptide produced and secreted by the cell. The host cell or stable host cell line can produce and secrete the prodrug composition described herein. In some instances, a suitable cell may produce an equimolar ratio (e.g., an about 1:1 ratio) of any one of the first polypeptides and any one of the second polypeptides described herein. In other embodiments, a suitable cell produces a non-equimolar ratio (e.g., a ratio that differs from 1:1) of any one of the first monomeric Fc polypeptides and any one of the second monomeric Fc polypeptides.

### V. Exemplary Formats of the Invention

As will be appreciated by those in the art, the heterodimeric protein compositions comprising two monomers that form a pro-drug composition can take on a wide variety of formats. What is important is that the active variable heavy domain and the active variable light domains each end up, post-cleavage, associated with an sdABD-TTA. That is, generally one sdABD-TTA is linked via a non-cleavable domain linker to the active variable heavy domain, and one sdABD-TTA is linked via a non-cleavable domain linker to the active variable light domain. This ensures that the active CD3 ABD can form on the tumor cell surface. Once cleavage occurs and the inactive VH and VL disassociate, the aVH and aVL intermolecularly associate to form one or more active CD3 ABDs.

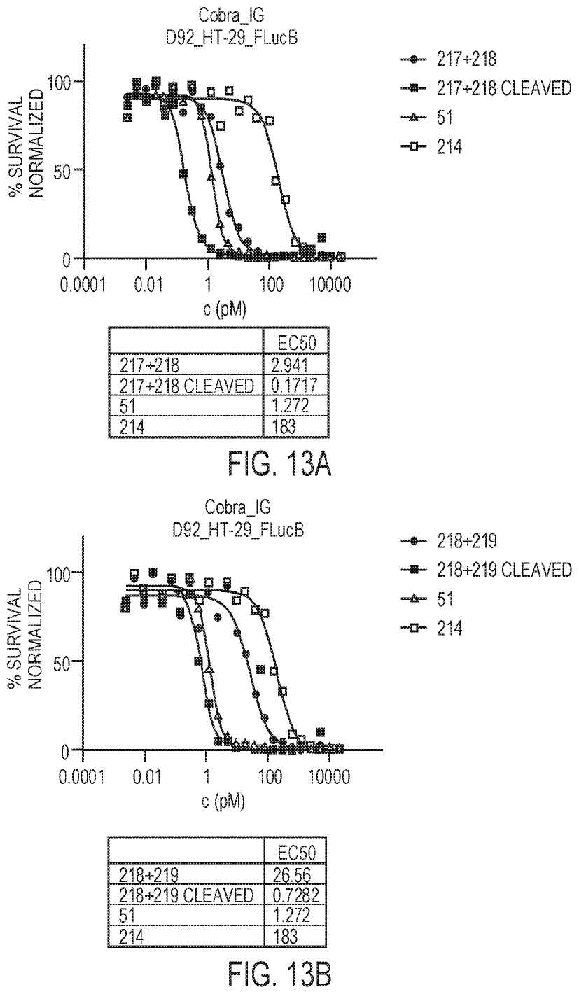

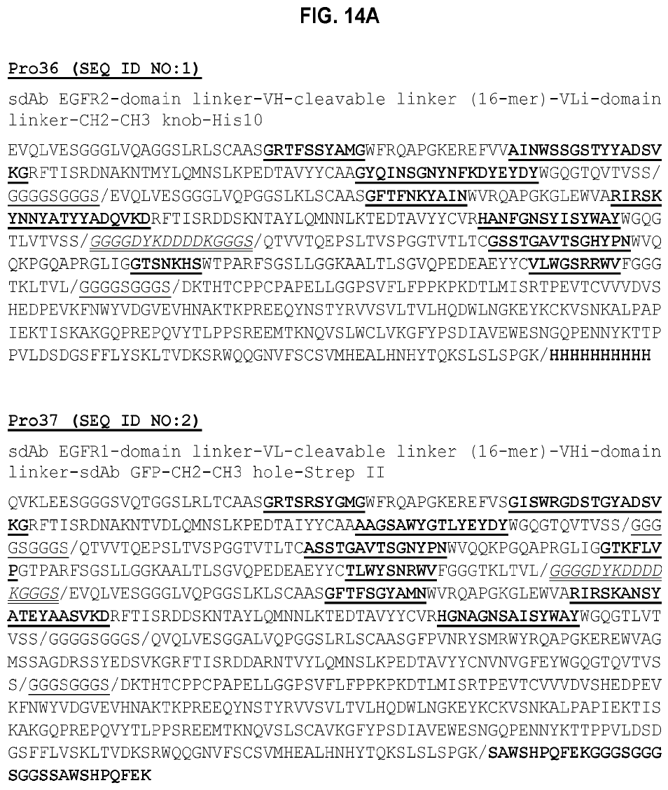

For all of the constructs and formats provided herein, a number of different components, e.g. sdABD-TTAs, cleavage sites, aVH and aVL domains, iVH and iVL domains, and Fc domains, such as all depicted in FIG. 13, can be “mixed and matched” in each format.

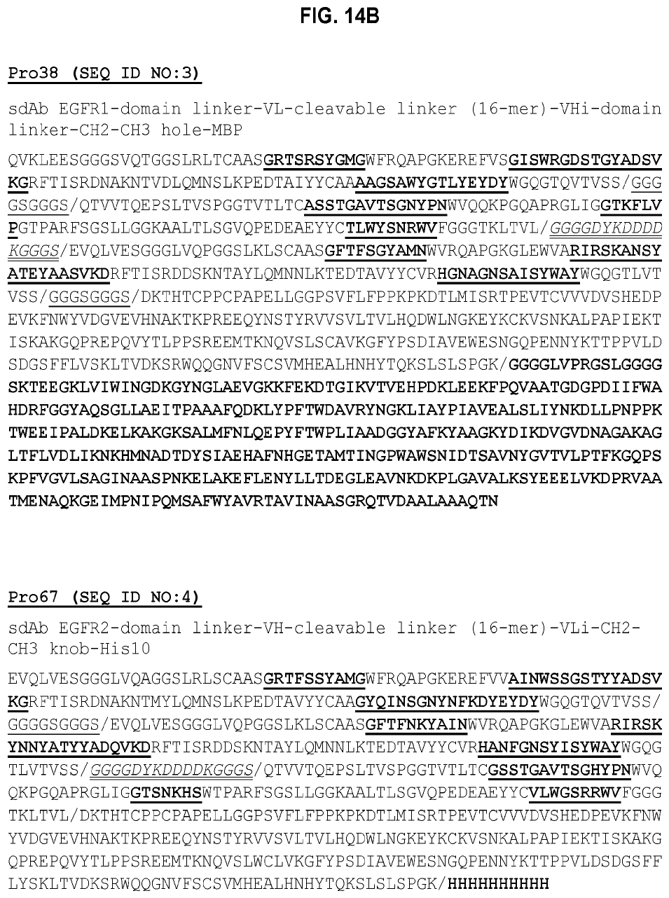

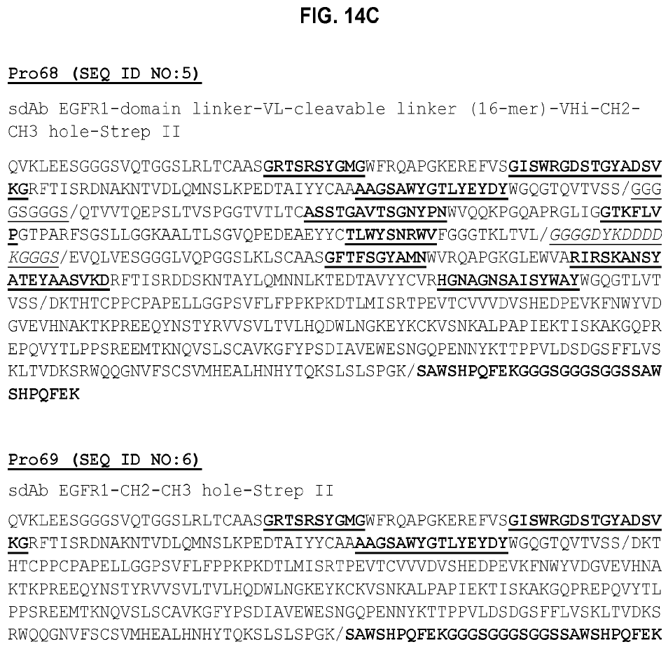

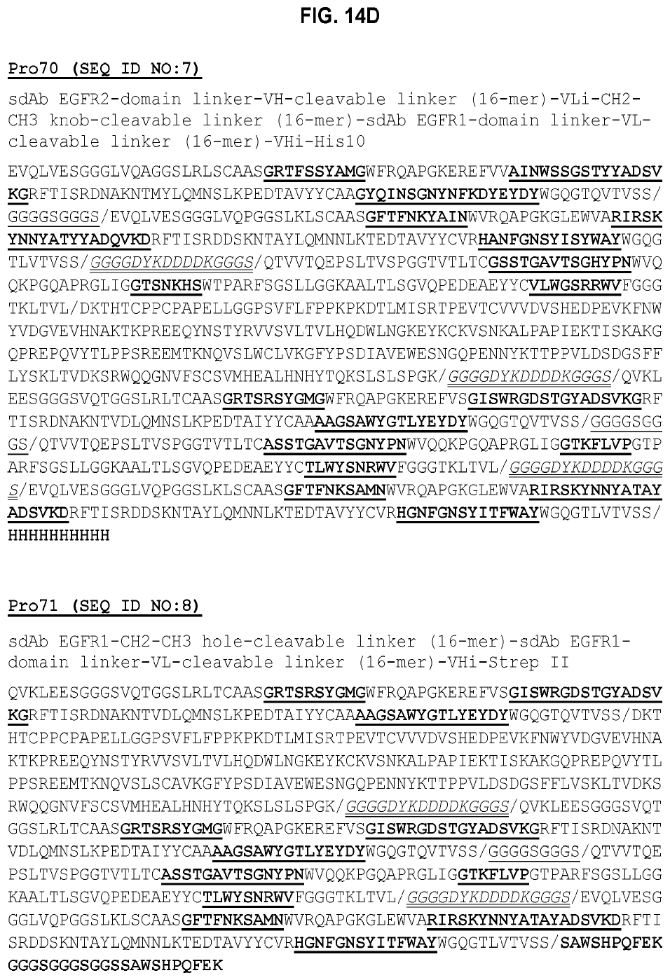

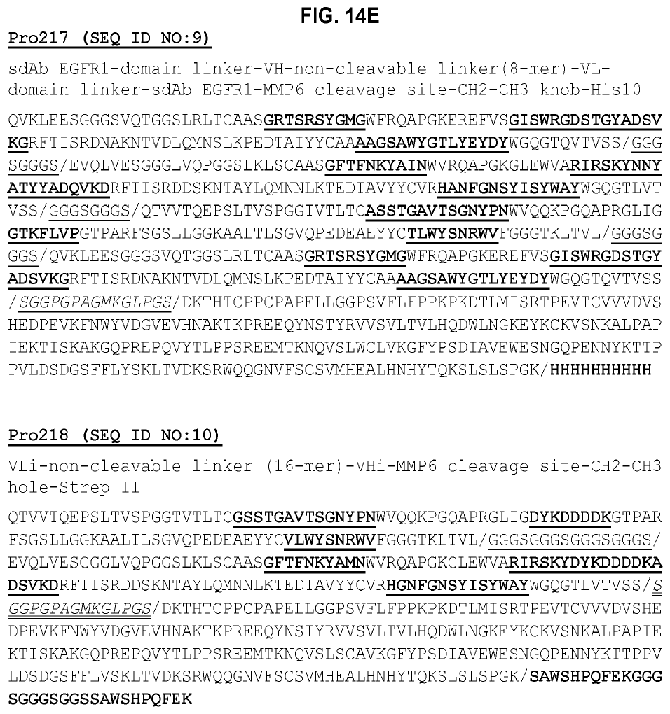

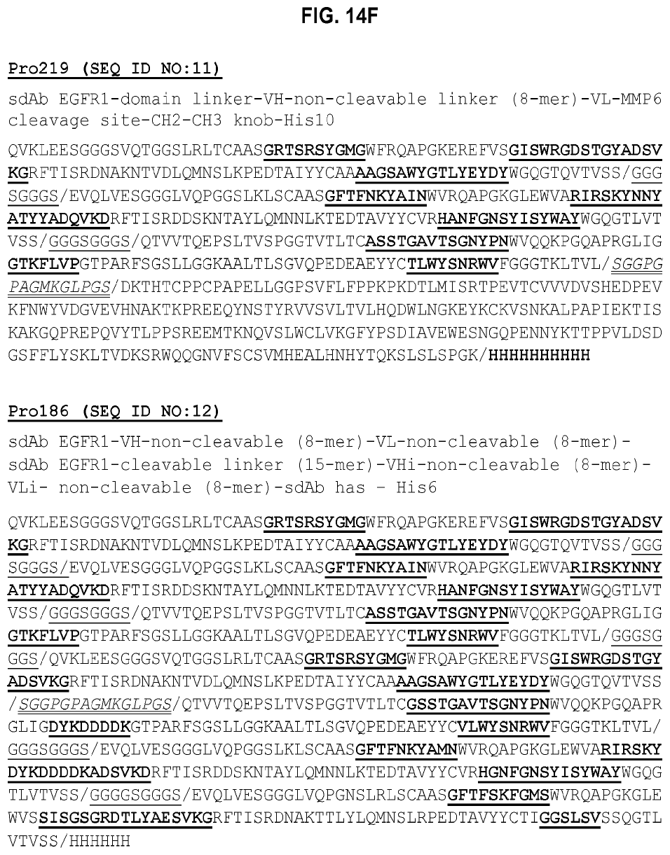

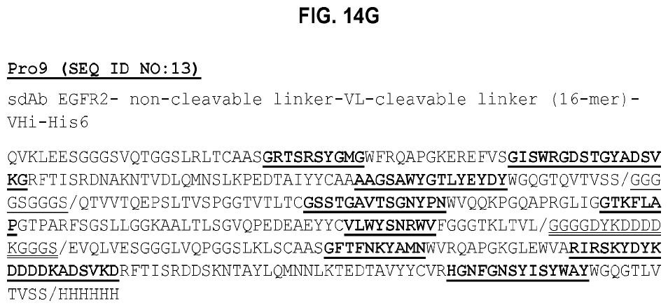

Provided herein are heterodimeric Fc fusion prodrug proteins (see, FIG. 4) comprising, a first monomeric Fc polypeptide comprising (from N- to C-terminal) an ABD against a TTA-domain linker-anti-CD3 pseudo Fv domain (e.g., an active VL domain-cleavable linker-inactive VH domain)-antigen binding domain against GFP-domain linker (hinge linker)-Fc hole; and a second monomeric Fc polypeptide comprising (from N- to C-terminal) an ABD against TTA-domain linker-anti-CD3 pseudo Fv domain (e.g., an active VH domain-cleavable linker-inactive VL domain)-domain linker (hinge linker)-Fc knob. In some embodiments, the first monomeric Fc polypeptide comprising (from N- to C-terminal) an ABD against a TTA-domain linker-anti-CD3 pseudo Fv domain (e.g., an active VH domain-cleavable linker-inactive VL domain)-antigen binding domain against GFP-domain linker (hinge linker)-Fc hole; and a second monomeric Fc polypeptide comprising (from N- to C-terminal) an ABD against TTA-domain linker-anti-CD3 pseudo Fv domain (e.g., an active VL domain-cleavable linker-inactive VH domain)-domain linker (hinge linker)-Fc knob. In some embodiments, the C-terminal end of the first monomeric Fc polypeptide includes a tag such as, but not limited to a (His)10 tag or a Strep-II tag, although this is generally not used for actual prodrug molecules to be administered to patients. In some embodiments, the C-terminal end of the second monomeric Fc polypeptide includes a tag such as, but not limited to a (His)10 tag or a Strep-II tag. In some embodiments, the prodrug construct includes a first monomeric Fc comprising sdABD(TTA)-NCL-active VL-CL-VHi-sdABD-NCL-Fc region comprising CH2-CH3 with a hole format, and a second monomeric Fc comprising sdABD(TTA)-NCL-active VH-CL-VLi-NCL-Fc region comprising CH2-CH3 with a knob format. In some embodiments, such heterodimeric Fc fusion prodrug proteins comprise Pro37 (SEQ ID NO:2) and Pro36 (SEQ ID NO:1), as depicted in FIG. 14A. The amino acid sequence of Pro37 is shown in FIG. 14A. The amino acid sequence of Pro37 is shown in FIG. 14A.

Also, provided herein are heterodimeric Fc fusion prodrug proteins (see. FIG. 5) comprising, a first monomeric Fc polypeptide comprising (from N- to C-terminal) an ABD against a TTA-domain linker-anti-CD3 pseudo Fv domain (e.g., an active VL domain-cleavable linker-inactive VH domain)-domain linker (hinge linker)-Fc hole; and a second monomeric Fc polypeptide comprising an ABD against TTA-domain linker-anti-CD3 pseudo Fv domain (e.g., an active VH domain-cleavable linker-inactive VL domain)-domain linker (hinge linker)-Fc knob. In some embodiments, the first monomeric Fc polypeptide comprising (from N- to C-terminal) an ABD against a TTA-domain linker-anti-CD3 pseudo Fv domain (e.g., an active VH domain-cleavable linker-inactive VL domain)-domain linker (hinge linker)-Fc hole; and a second monomeric Fc polypeptide comprising (from N- to C-terminal) an ABD against TTA-domain linker-anti-CD3 pseudo Fv domain (e.g., an active VL domain-cleavable linker-inactive VH domain)-domain linker (hinge linker)-Fc knob. In some embodiments, the C-terminal end of the first monomeric Fc polypeptide includes a tag such as, but not limited to a (His)10 tag or a Strep-II tag. In some embodiments, the C-terminal end of the second monomeric Fc polypeptide includes a tag such as, but not limited to a (His)10tag or a Strep-II tag. In some embodiments, such heterodimeric Fc fusion prodrug proteins comprise Pro38 and Pro36, as depicted in FIG. 5. The amino acid sequence of Pro36 is depicted in FIG. 14A. The amino acid sequence of Pro38 is depicted in FIG. 14B.

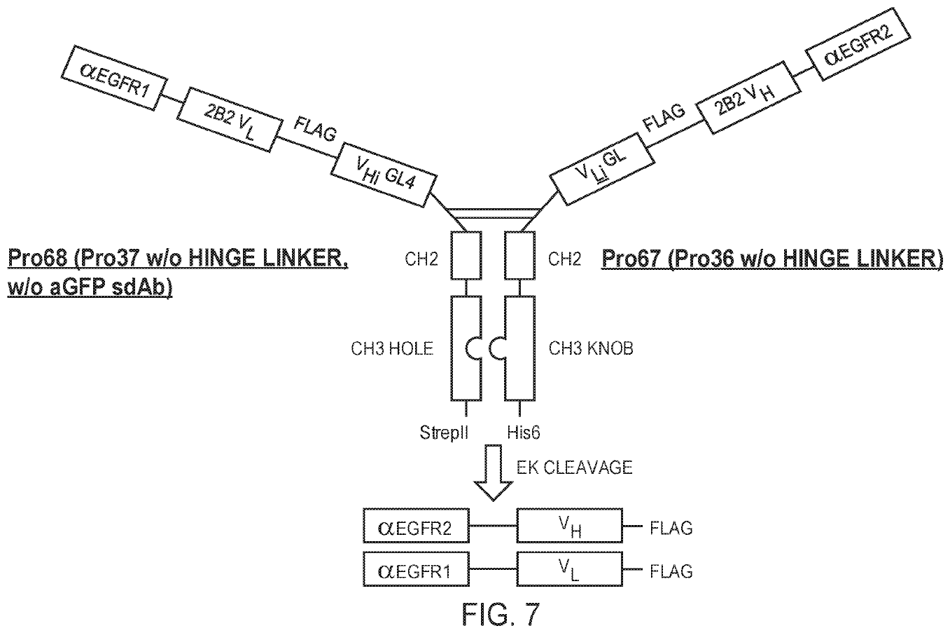

Provided herein are heterodimeric Fc fusion prodrug proteins (see, FIG. 7) comprising, a first monomeric Fc polypeptide comprising (from N- to C-terminal) an ABD against a TTA-domain linker-anti-CD3 pseudo Fv domain (e.g., an active VL domain-cleavable linker-inactive VH domain)-Fc hole; and a second monomeric Fc polypeptide comprising (from N- to C-terminal) an ABD against TTA-domain linker-anti-CD3 pseudo Fv domain (e.g., an active VH domain-cleavable linker-inactive VL domain)-Fc knob. In some embodiments, the first monomeric Fc polypeptide comprising (from N- to C-terminal) an ABD against a TTA-domain linker-anti-CD3 pseudo Fv domain (e.g., an active VH domain-cleavable linker-inactive VL domain)-Fc hole; and a second monomeric Fc polypeptide comprising (from N- to C-terminal) an ABD against TTA-domain linker-anti-CD3 pseudo Fv domain (e.g., an active VL domain-cleavable linker-inactive VH domain)-Fc knob. In some embodiments, the C-terminal end of the first monomeric Fc polypeptide includes a tag such as, but not limited to a (His)10 tag or a Strep-II tag. In some embodiments, the C-terminal end of the second monomeric Fc polypeptide includes a tag such as, but not limited to a (His)10 tag or a Strep-II tag. In some embodiments, such heterodimeric Fc fusion prodrug proteins comprise Pro68 (SEQ ID NO:5) and Pro67 (SEQ ID NO:4), as depicted in FIG. 14A-14B. Pro68 resembles Pro37 but does not include a sdABD that binds GFP or a domain linker attached to the CH2 domain. Pro67 resembles Pro36 but does not include a domain linker attached to the CH2 domain. The amino acid sequence of Pro68 is depicted in FIG. 14C. The amino acid sequence of Pro67 is depicted in FIG. 14B.

Provided herein are heterodimeric Fc fusion prodrug proteins (see, FIG. 8) comprising, a first monomeric Fc polypeptide comprising (from N- to C-terminal) an ABD against a TTA-Fc hole; and a second monomeric Fc polypeptide comprising (from N- to C-terminal) an ABD against a TTA-domain linker-anti-CD3 pseudo Fv domain (e.g., an active VH domain-cleavable linker-inactive VL domain)-Fc knob-cleavable linker-an ABD against a TTA-domain linker-anti-CD3 pseudo Fv domain (e.g., an active VL domain-cleavable linker-inactive VH domain). In some embodiments, the first monomeric Fc polypeptide comprising (from N- to C-terminal) an ABD against a TTA-Fc hole; and a second monomeric Fc polypeptide comprising (from N- to C-terminal) an ABD against a TTA-domain linker-anti-CD3 pseudo Fv domain (e.g., an active VL domain-cleavable linker-inactive VH domain)-Fc knob-cleavable linker-an ABD against a TTA-domain linker-anti-CD3 pseudo Fv domain (e.g., an active VH domain-cleavable linker-inactive VL domain). In some embodiments, the C-terminal end of the first monomeric Fc polypeptide includes a tag such as, but not limited to a (His)10tag or a Strep-II tag. In some embodiments, the C-terminal end of the second monomeric Fc polypeptide includes a tag such as, but not limited to a (His)10 tag or a Strep-II tag. In some embodiments, such heterodimeric Fc fusion prodrug proteins comprise Pro69 (SEQ ID NO:6) and Pro70 (SEQ ID NO:7), as depicted in FIG. 14C-14D. Pro70 resembles Pro67 with a Pro9 construct attached at the C-terminus. The amino acid sequence of Pro69 is depicted in FIG. 14C. The amino acid sequence of Pro70 is depicted in FIG. 14D.

Provided herein are heterodimeric Fc fusion prodrug proteins (see, FIG. 9) comprising, a first monomeric Fc polypeptide comprising (from N- to C-terminal) an ABD against a TTA-Fc hole-cleavable linker-ABD against a TTA-domain linker-anti-CD3 pseudo Fv domain (e.g., active VL domain-cleavable linker-inactive VH domain); and a second monomeric Fc polypeptide comprising (from N- to C-terminal) an ABD against a TTA-domain linker-anti-CD3 pseudo Fv domain (e.g., an active VH domain-cleavable linker-inactive VL domain)-Fc knob. In some embodiments, the first monomeric Fc polypeptide comprising (from N- to C-terminal) an ABD against a TTA-Fc hole-cleavable linker-ABD against a TTA-domain linker-anti-CD3 pseudo Fv domain (e.g., active VH domain-cleavable linker-inactive VL domain); and a second monomeric Fc polypeptide comprising (from N- to C-terminal) an ABD against a TTA-domain linker-anti-CD3 pseudo Fv domain (e.g., an active VL domain-cleavable linker-inactive VH domain)-Fc knob. In some embodiments, the C-terminal end of the first monomeric Fc polypeptide includes a tag such as, but not limited to a (His)10 tag or a Strep-II tag. In some embodiments, the C-terminal end of the second monomeric Fc polypeptide includes a tag such as, but not limited to a (His)10 tag or a Strep-II tag. In some embodiments, such heterodimeric Fc fusion prodrug proteins comprise Pro71 (SEQ ID NO:8) and Pro67 (SEQ ID NO:4), as depicted in FIG. 14B and FIG. 14D. Pro71 resembles Pro69 with a Pro9 construct attached at the C-terminus. Pro67 resembles Pro36 without a domain linker attached to the CH2 domain. The amino acid sequence of Pro71 is depicted in FIG. 14D. The amino acid sequence of Pro67 is depicted in FIG. 14B.

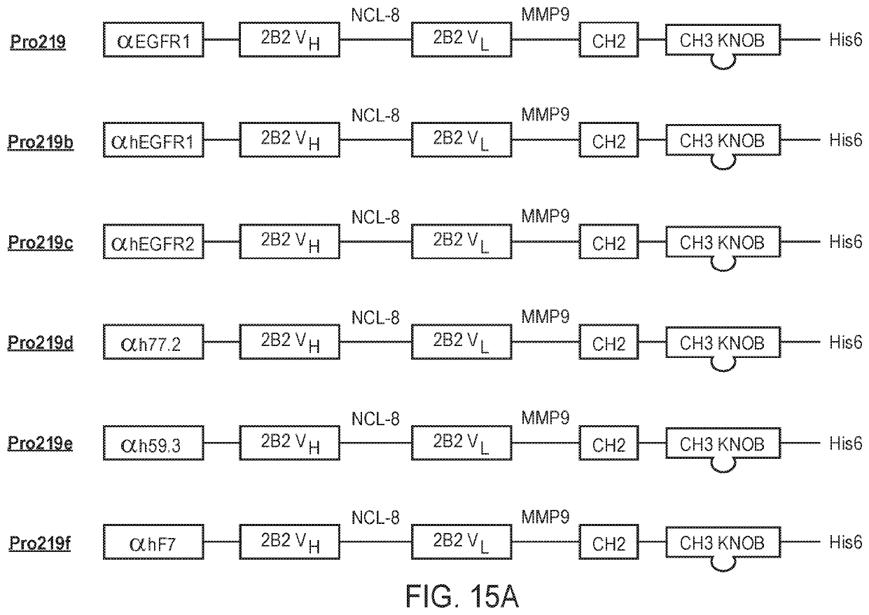

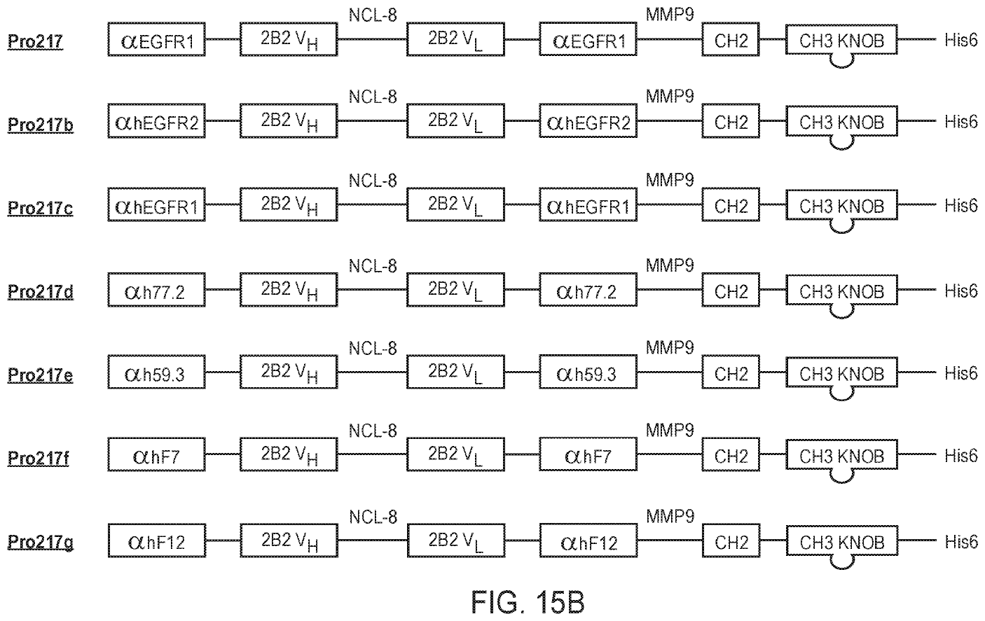

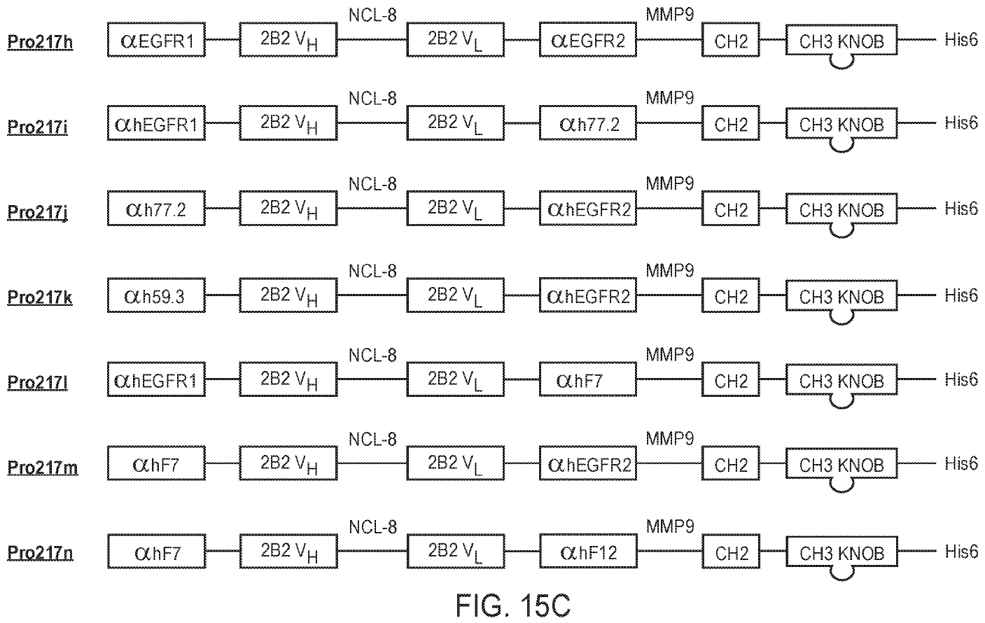

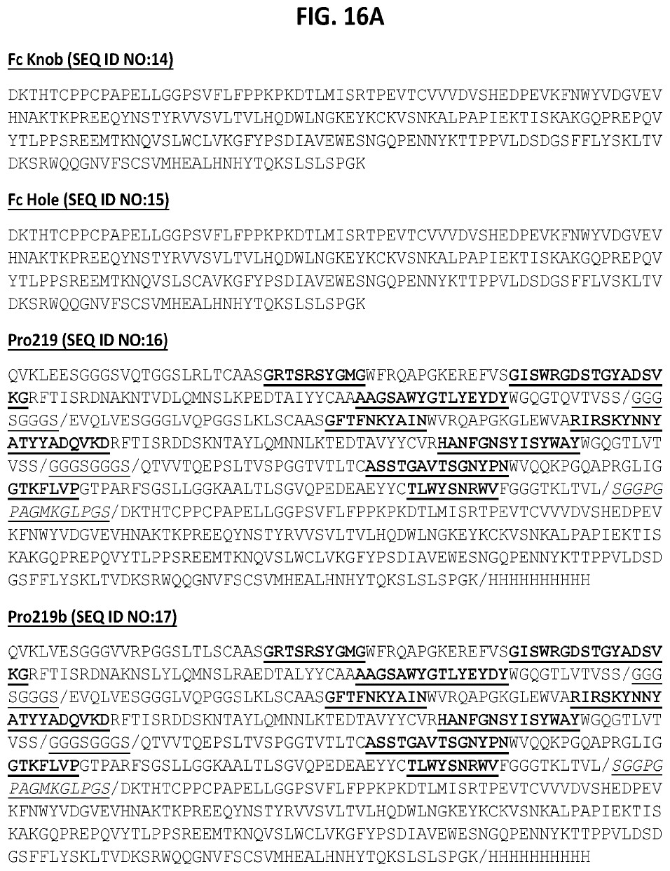

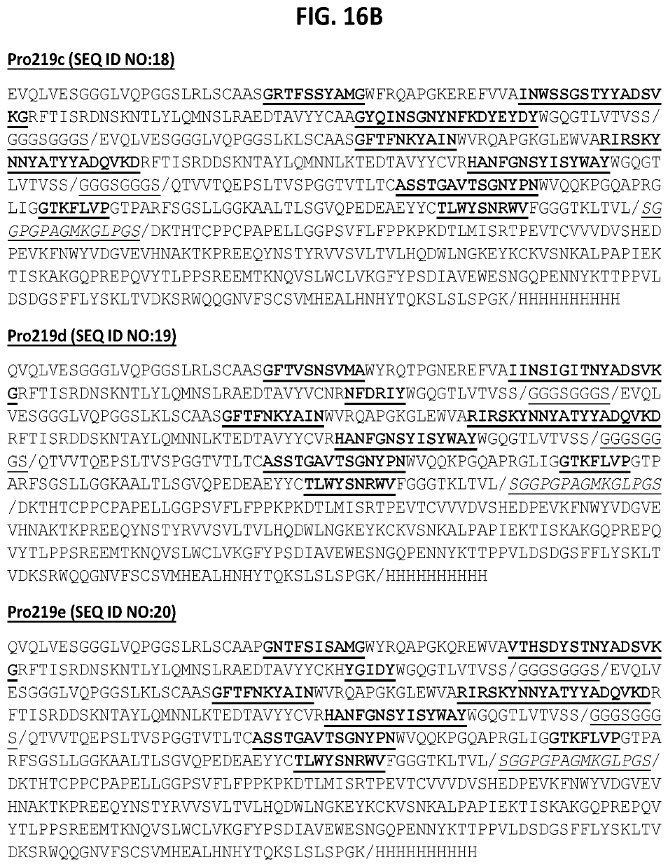

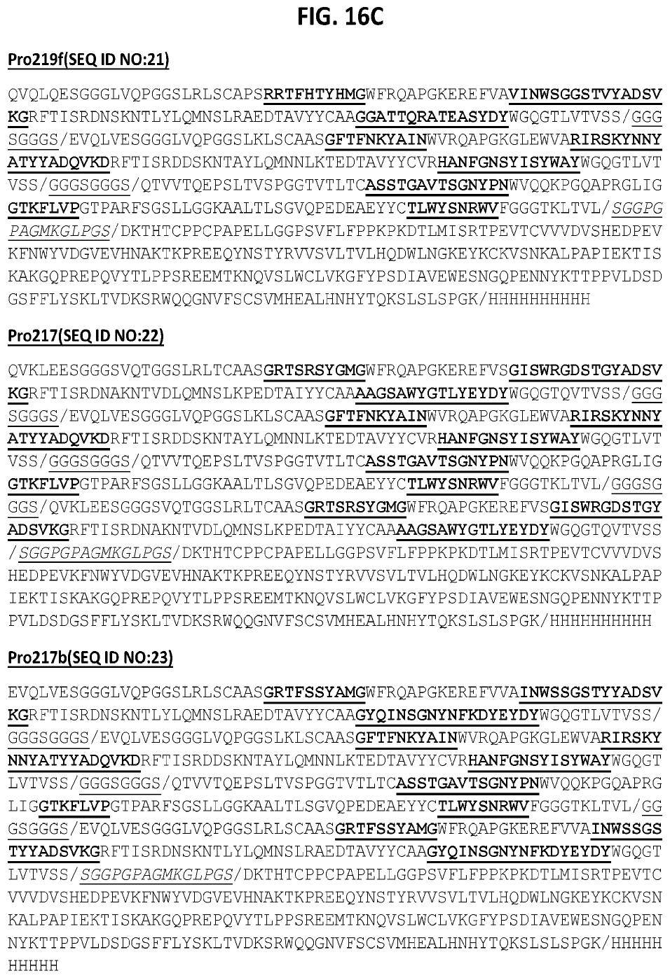

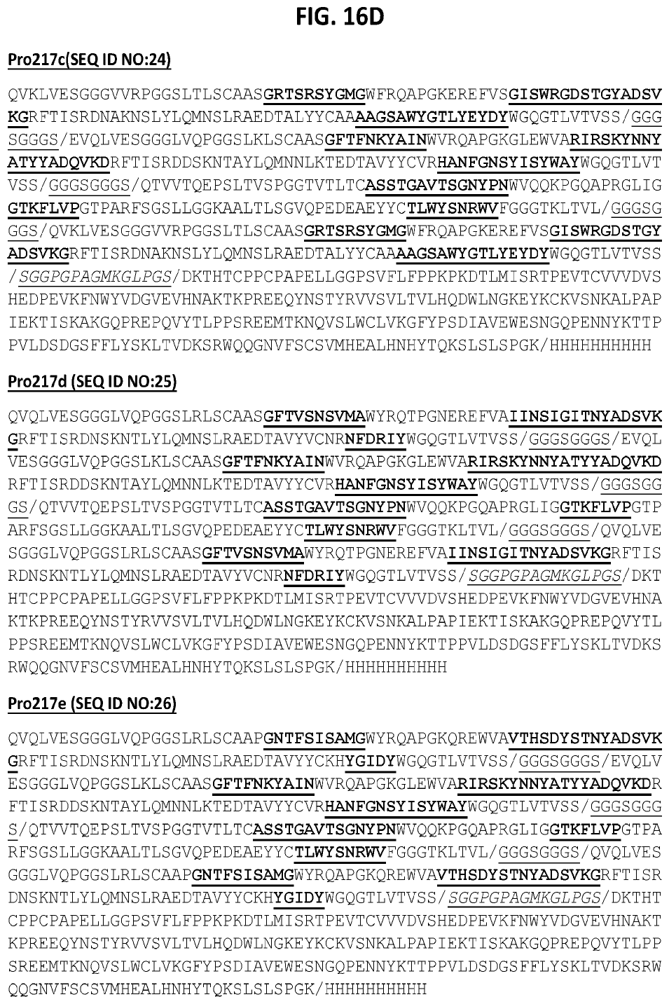

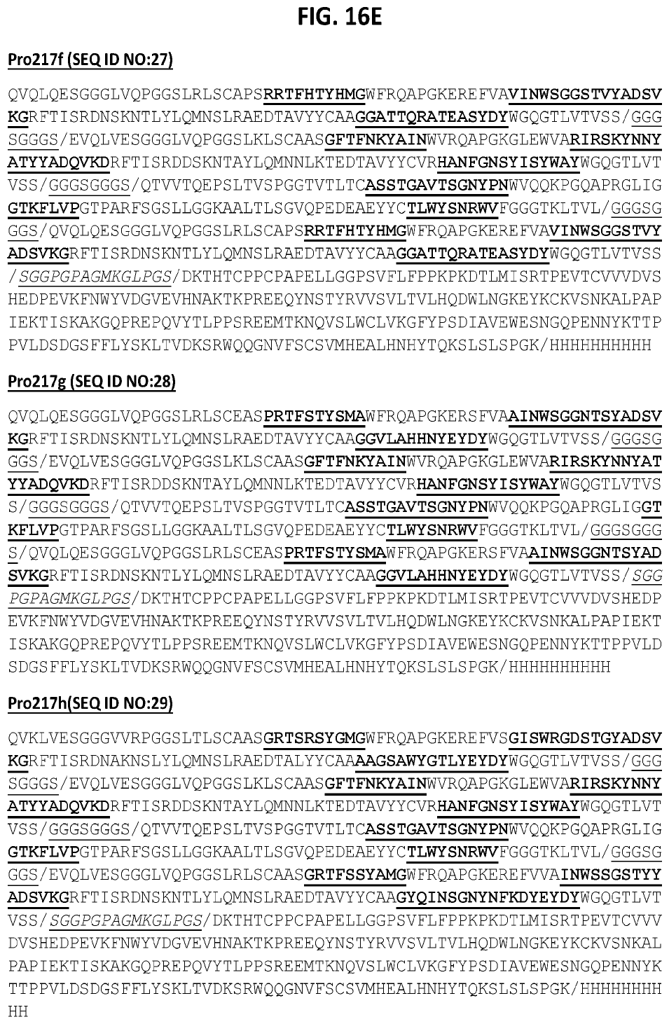

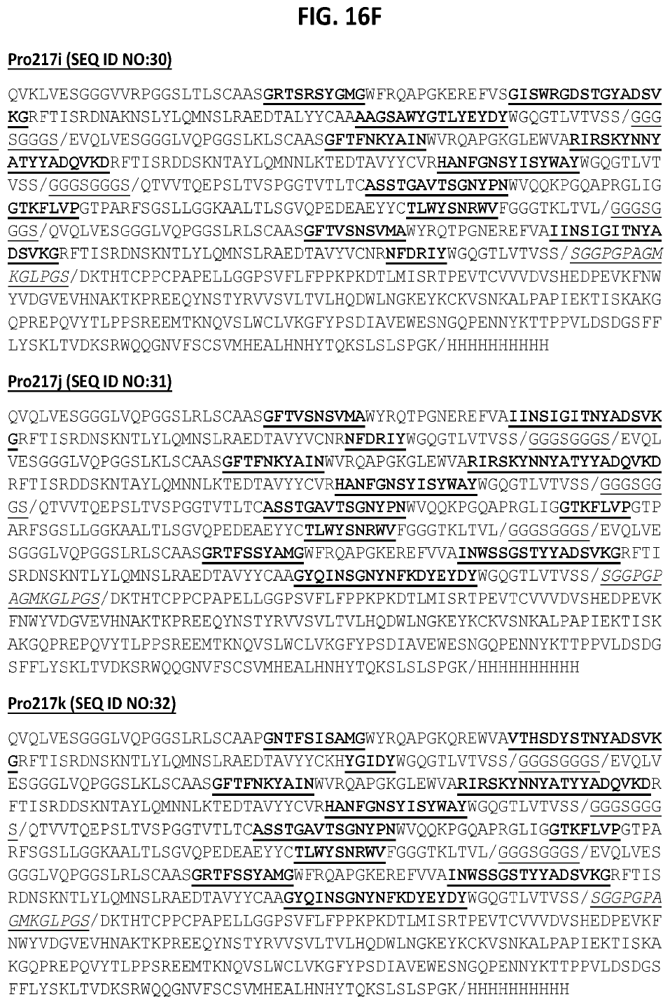

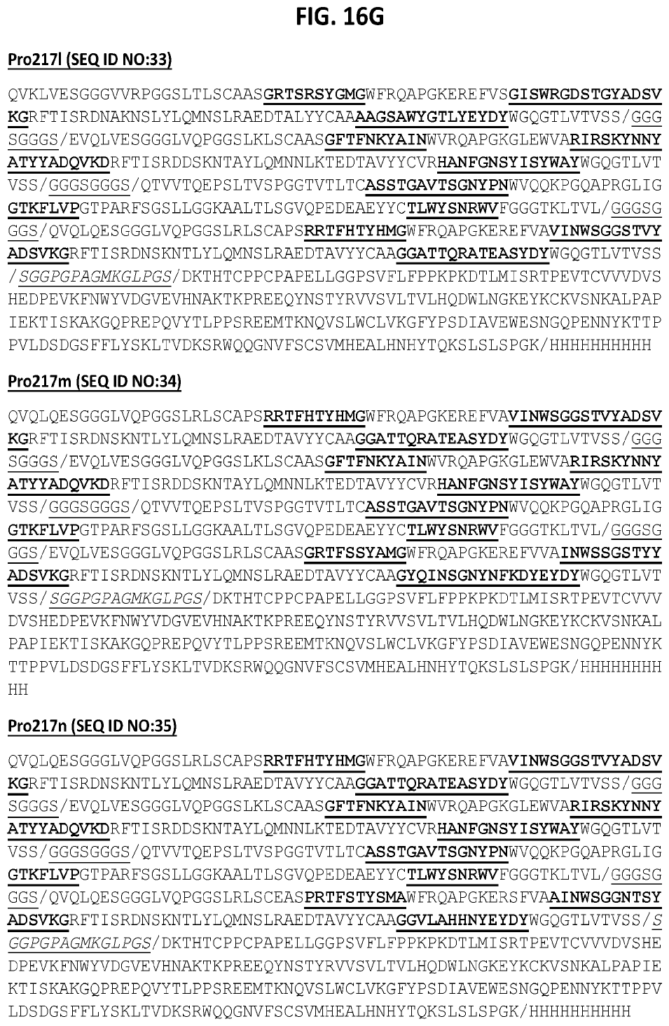

Provided herein are heterodimeric Fc fusion prodrug proteins (see, FIG. 11) comprising, a first monomeric Fc polypeptide comprising (from N- to C-terminal) an ABD against a TTA-domain linker-constrained anti-CD3 Fv domain (e.g.; active VH domain-non-cleavable constrained linker (NCCL)-active VL domain)-cleavable linker-Fc knob, and a second monomeric Fc polypeptide comprising (from N- to C-terminal) an anti-CD3 pseudo Fv domain (e.g., an inactive VL domain-non-cleavable linker-inactive VH domain)-cleavable linker-Fc hole. In some embodiments, the first monomeric Fc polypeptide comprising (from N- to C-terminal) an ABD against a TTA-domain linker-constrained anti-CD3 Fv domain (e.g., active VL domain-non-cleavable constrained linker (NCCL)-active VH domain)-cleavable linker-Fc knob, and a second monomeric Fc polypeptide comprising (from N- to C-terminal) an anti-CD3 pseudo Fv domain (e.g., an inactive VH domain-non-cleavable linker-inactive VL domain)-cleavable linker-Fc hole. In some embodiments, the C-terminal end of the first monomeric Fc polypeptide includes a tag such as, but not limited to a (His)10 tag or a Strep-II tag. In some embodiments, the C-terminal end of the second monomeric Fc polypeptide includes a tag such as, but not limited to a (His)10 tag or a Strep-II tag. In some embodiments, such heterodimeric Fc fusion prodrug proteins comprise Pro219 (SEQ ID NO:9 or 16) and Pro218 (SEQ ID NO:10), as depicted in FIG. 11. The amino acid sequence of Pro218 is depicted in FIG. 14E. The amino acid sequence of Pro219 is depicted in FIG. 14E. In some embodiments, the first monomeric Fc component comprises an ABD such as but not limited to those provided in Pro219, Pro219b, Pro219c, Pro219d, Pro219e, and Pro219f, as depicted in FIG. 15A and FIG. 16A-16C and SEQ ID NOS:16-21.

Provided herein are heterodimeric Fc fusion prodrug proteins (see, FIG. 12) comprising, a first monomeric Fc polypeptide comprising (from N- to C-terminal) an ABD against a TTA-domain linker-constrained anti-CD3 Fv domain (e.g., active VH domain-non-cleavable constrained linker (NCCL)-active VL domain)-an ABD against a TTA-cleavable linker-Fc knob, and a second monomeric Fc polypeptide comprising (from N- to C-terminal) an anti-CD3 pseudo Fv domain (e.g., an inactive VL domain-non-cleavable linker-inactive VH domain)-cleavable linker-Fc hole. In some embodiments, the first monomeric Fc polypeptide comprising (from N- to C-terminal) an ABD against a TTA-domain linker-constrained anti-CD3 Fv domain (e.g., active VL domain-non-cleavable constrained linker (NCCL)-active VH domain)-an ABD against a TTA-cleavable linker-Fc knob, and a second monomeric Fc polypeptide comprising (from N- to C-terminal) an anti-CD3 pseudo Fv domain (e.g., an inactive VH domain-non-cleavable linker-inactive VL domain)-cleavable linker-Fc hole. In some embodiments, the C-terminal end of the first monomeric Fc polypeptide includes a tag such as, but not limited to a (His)10 tag or a Strep-II tag. In some embodiments, the C-terminal end of the second monomeric Fc polypeptide includes a tag such as, but not limited to a (His)10 tag or a Strep-II tag. In some embodiments, such heterodimeric Fc fusion prodrug proteins comprise Pro217 (SEQ ID NO:9 or 22) and Pro218 (SEQ ID NO:10), as depicted in FIG. 12. The amino acid sequence of Pro218 is depicted in FIG. 14E. The amino acid sequence of Pro217 is depicted in FIG. 14E. In some embodiments, the first monomeric Fc component comprises an ABD such as but not limited to those provided in Pro217, Pro217b, Pro217c, Pro217d, Pro217e, Pro217f, Pro217g, Pro217h, Pro217i, Pro217j, Pro217k, Pro217l, Pro217m, and Pro217n, as depicted in FIG. 15B-15C and FIG. 16C-16G and SEQ ID NOS:22-35.

The ABDs against TTAs of the heterodimeric Fc prodrug constructs can be single domain antibodies that bind TTAs. In some embodiments, the single domain antibody (sdABD) is an sdABD against EGFR, an sdABD against EpCAM, an sdABD against another target tumor antigen. In some embodiments of the heterodimeric Fc fusion prodrug proteins, the sdABD of the first monomeric Fc and the sdABD of the second monomeric Fc have the same or substantially the same amino acid sequence. In some embodiments, the sdABD of the first monomeric Fc and the sdABD of the second monomeric Fc bind the same TTA. In other embodiments, the sdABD of the first monomeric Fc and the sdABD of the second monomeric Fc bind different TTAs. In other instances, the sdABD of the first monomeric Fc and the sdABD of the second monomeric Fc have different amino acid sequences. In some embodiments, the sdABD has CDRs and/or variable domains of any sdABDs set forth in SEQ ID NOS:50-169, of FIG. 1A-1G.

In some embodiments of the CD3 binding domains, the active VH and active VL have the sequences of CD3 VH and CD3 VL and the VHi and VLi have the sequences of of CD3 VHi and CD3 VLi, of FIG. 2A-2B. In some embodiments of the CD3 binding domains, the active VH and active VL have the sequences of CD3 VH and CD3 VL and the VHi and VLi have the sequences of CD3 VHi2 and CD3 VLi2, of FIG. 2A-2B. In some embodiments of the CD3 binding domains, the active VH and active VL have the sequences of CD3 VH and CD3 VL and the VHi and VLi have the sequences of CD3 VHiGL4 and CD3 VLiGL, of FIG. 2A-2B. In some instances, the pseudo Fv domain of the first monomeric Fc protein can comprise a VL and a VHi linked using a cleavable linker, either (N- to C-terminal) VL-linker-VHi or VHi-linker-VL. In some embodiments, the pseudo Fv domain has the structure (N- to C-terminus) of vlFR1-vlCDR1-vlFR2-vlCDR2-vlFR3-vlCDR3-vlFR4-CL-vhiFR1-vhiCDR1-vhiFR2-vhiCDR2-vhiFR3-vhiCDR3-vhiFR4. In other instances, the pseudo Fv domain has the structure (N- to C-terminus) of vhiFR1-vhiCDR1-vhiFR2-vhiCDR2-vhiFR3-vhiCDR3-vhiFR4-CL-vlFR1-vlCDR1-vlFR2-vlCDR2-vlFR3-vlCDR3-vlFR4. In some embodiments, the pseudo Fv domain of the second monomeric Fc protein can comprise a VH and a VLi linked using a cleavable linker, either (N- to C-terminal) VH-linker-VLi or VLi-linker-VH. In some instances, the pseudo Fv domain has the structure (N- to C-terminus) of vhFR1-vhCDR1-vhFR2-vhCDR2-vhFR3-vhCDR3-vhFR4-CL-vliFR1-vliCDR1-vliFR2-vliCDR2-vliFR3-vliCDR3-vliFR4. In other instances, the pseudo Fv domain has the structure (N- to C-terminus) of vliFR1-vliCDR1-vliFR2-vliCDR2-vliFR3-vliCDR3-vliFR4-CL-vhFR1-vhCDR1-vhFR2-vhCDR2-vhFR3-vhCDR3-vhFR4.

In some embodiments, the present invention provides constrained Fv domains, that comprise an active VH and an active VL domain that are covalently attached using a constrained linker (which, as outlined herein, can be cleavable or non-cleavable). The constrained linker prevents intramolecular association between the VH and VL in the absence of cleavage. Thus, a constrained Fv domain comprises a set of six CDRs contained within variable domains, wherein the vhCDR1, vhCDR2 and vhCDR3 of the VH bind human CD3 and the vlCDR1, vlCDR2 and vlCDR3 of the VL bind human CD3, but in the prodrug format (e.g., uncleaved), the VH and VL are unable to sterically associate to form an active binding domain.

The constrained Fv domains can comprise active VH and active VL (VHa and VLa) or inactive VH and VL (VHi and VLi). As will be appreciated by those in the art, the order of the VH and VL in a constrained active Fv domain can be either (N- to C-terminal) VH-linker-VL or VL-linker-VH. As outlined herein, the constrained active Fv domains can comprise a VH and a VL linked using a non-cleavable linker, in cases such as those shown as Pro219 or Pro217. In this embodiment, the constrained Fv domain has the structure (N- to C-terminus) of vhFR1-vhCDR1-vhFR2-vhCDR2-vhFR3-vhCDR3-vhFR4-CCL-vlFR1-vlCDR1-vlFR2-vlCDR2-vlFR3-vlCDR3-vlFR4. In this embodiment, the CDRs and/or variable domains are those of active CD3 VH and active CD3VL as provided as SEQ ID NOS:186 and 170, respectively.

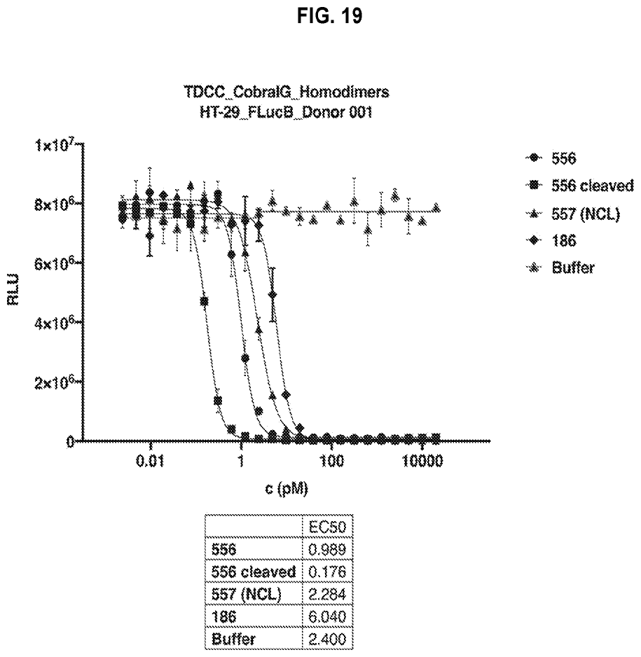

Provided herein are homodimeric Fc fusion prodrug proteins (see, FIG. 17A such as Pro556) comprising, a first monomeric Fc polypeptide comprising (from N- to C-terminal) an ABD against a TTA-domain linker-constrained anti-CD3 Fv domain (e.g., active VH domain-non-cleavable constrained linker (NCCL)-active VL domain)-an ABD against a TTA-cleavable linker-an anti-CD3 pseudo Fv domain (e.g., an inactive VL domain-noncleavable linker-inactive VH domain)-Fc domain and a second monomeric Fc polypeptide comprising (from N- to C-terminal) an ABD against a TTA-domain linker-constrained anti-CD3 Fv domain (e.g., active VH domain-non-cleavable constrained linker (NCCL)-active VL domain)-an ABD against a TTA-cleavable linker-an anti-CD3 pseudo Fv domain (e.g., an inactive VL domain-noncleavable linker-inactive VH domain)-Fc domain. In some embodiments, the C-terminal end of the first monomeric Fc polypeptide includes a tag such as, but not limited to a (His)10 tag or a Strep-II tag. In some embodiments, the C-terminal end of the second monomeric Fc polypeptide includes a tag such as, but not limited to a (His)10 tag or a Strep-II tag. In some embodiments, such homodimeric Fc fusion prodrug proteins comprise Pro556 and Pro556. The amino acid sequence of Pro556 is depicted in FIG. 17A. The data depicted in FIG. 19 illustrates the conditionality and activity of the homodimeric Pro556 construct. In some embodiments, any of the ABDs against a TTA can be used in the homodimeric Fc protein. In one embodiment, the sdABD is selected from the group consisting of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166.

Provided herein are non-cleavable homodimeric Fc fusion prodrug proteins (see. FIG. 17A such as Pro557) comprising, a first monomeric Fc polypeptide comprising (from N- to C-terminal) an ABD against a TTA-domain linker-constrained anti-CD3 Fv domain (e.g., active VH domain-non-cleavable constrained linker (NCCL)-active VL domain)-an ABD against a TTA-noncleavable linker (NCL-15)-an anti-CD3 pseudo Fv domain (e.g., an inactive VL domain-noncleavable linker-inactive VH domain)-Fc domain and a second monomeric Fc polypeptide comprising (from N- to C-terminal) an ABD against a TTA-domain linker-constrained anti-CD3 Fv domain (e.g., active VH domain-non-cleavable constrained linker (NCCL)-active VL domain)-an ABD against a TTA-noncleavable linker (NCL-15)-an anti-CD3 pseudo Fv domain (e.g., an inactive VL domain-noncleavable linker-inactive VH domain)-Fc domain. Such homodimeric Fc fusion proteins can act as a non-cleavable control. In some embodiments, the C-terminal end of the first monomeric Fc polypeptide includes a tag such as, but not limited to a (His)10 tag or a Strep-II tag. In some embodiments, the C-terminal end of the second monomeric Fc polypeptide includes a tag such as, but not limited to a (His)10 tag or a Strep-II tag. In some embodiments, such homodimeric Fc fusion prodrug proteins comprise Pro557 and Pro557. The amino acid sequence of Pro557 is depicted in FIG. 17A. Homodimeric Pro557 prodrug construct are not cleaved by a protease. In some embodiments, any of the ABDs against a TTA can be used in the homodimeric Fc protein. In one embodiment, the sdABD is selected from the group consisting of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166.

Provided herein are homodimeric Fc fusion prodrug proteins (see, FIG. 17B such as Pro587) comprising, a first monomeric Fc polypeptide comprising (from N- to C-terminal) an ABD against a TTA-domain linker-constrained anti-CD3 Fv domain (e.g., active VL domain-non-cleavable constrained linker (NCCL)-active VH domain)-an ABD against a TTA-cleavable linker-an anti-CD3 pseudo Fv domain (e.g., an inactive VL domain-noncleavable linker-inactive VH domain)-Fc domain and a second monomeric Fc polypeptide comprising (from N- to C-terminal) an ABD against a TTA-domain linker-constrained anti-CD3 Fv domain (e.g., active VL domain-non-cleavable constrained linker (NCCL)-active VH domain)-an ABD against a TTA-cleavable linker-an anti-CD3 pseudo Fv domain (e.g., an inactive VL domain-noncleavable linker-inactive VH domain)-Fc domain. In some embodiments, the C-terminal end of the first monomeric Fc polypeptide includes a tag such as, but not limited to a (His)10 tag or a Strep-II tag. In some embodiments, the C-terminal end of the second monomeric Fc polypeptide includes a tag such as, but not limited to a (His)10 tag or a Strep-II tag. In some embodiments, such homodimeric Fc fusion prodrug proteins comprise Pro587 and Pro587. The amino acid sequence of Pro587 is depicted in FIG. 17B. In some embodiments, any of the ABDs against a TTA can be used in the homodimeric Fc protein. In one embodiment, the sdABD is selected from the group consisting of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166.

Provided herein are homodimeric Fc fusion prodrug proteins (see, FIG. 17B such as Pro588) comprising, a first monomeric Fc polypeptide comprising (from N- to C-terminal) an ABD against a TTA-domain linker-constrained anti-CD3 Fv domain (e.g., active VH domain-non-cleavable constrained linker (NCCL)-active VL domain)-an ABD against a TTA-cleavable linker-an anti-CD3 pseudo Fv domain (e.g., an inactive VH domain-noncleavable linker-inactive VL domain)-Fc domain and a second monomeric Fc polypeptide comprising (from N- to C-terminal) an ABD against a TTA-domain linker-constrained anti-CD3 Fv domain (e.g., active VH domain-non-cleavable constrained linker (NCCL)-active VL domain)-an ABD against a TTA-cleavable linker-an anti-CD3 pseudo Fv domain (e.g., an inactive VH domain-noncleavable linker-inactive VL domain)-Fc domain. In some embodiments, the C-terminal end of the first monomeric Fc polypeptide includes a tag such as, but not limited to a (His)10 tag or a Strep-II tag. In some embodiments, the C-terminal end of the second monomeric Fc polypeptide includes a tag such as, but not limited to a (His)10 tag or a Strep-II tag. In some embodiments, such homodimeric Fc fusion prodrug proteins comprise Pro588 and Pro588. The amino acid sequence of Pro588 is depicted in FIG. 17B. In some embodiments, any of the ABDs against a TTA can be used in the homodimeric Fc protein. In one embodiment, the sdABD is selected from the group consisting of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166.

Provided herein are homodimeric Fc fusion prodrug proteins (see, FIG. 17C such as Pro589) comprising, a first monomeric Fc polypeptide comprising (from N- to C-terminal) an ABD against a TTA-domain linker-constrained anti-CD3 Fv domain (e.g., active VL domain-non-cleavable constrained linker (NCCL)-active VH domain)-an ABD against a TTA-cleavable linker-an anti-CD3 pseudo Fv domain (e.g., an inactive VH domain-noncleavable linker-inactive VL domain)-Fc domain and a second monomeric Fc polypeptide comprising (from N- to C-terminal) an ABD against a TTA-domain linker-constrained anti-CD3 Fv domain (e.g., active VL domain-non-cleavable constrained linker (NCCL)-active VH domain)-an ABD against a TTA-cleavable linker-an anti-CD3 pseudo Fv domain (e.g., an inactive VH domain-noncleavable linker-inactive VL domain)-Fc domain. In some embodiments, the C-terminal end of the first monomeric Fc polypeptide includes a tag such as, but not limited to a (His)10 tag or a Strep-II tag. In some embodiments, the C-terminal end of the second monomeric Fc polypeptide includes a tag such as, but not limited to a (His)10 tag or a Strep-II tag. In some embodiments, such homodimeric Fc fusion prodrug proteins comprise Pro589 and Pro589. The amino acid sequence of Pro589 is depicted in FIG. 17C. In some embodiments, any of the ABDs against a TTA can be used in the homodimeric Fc protein. In one embodiment, the sdABD is selected from the group consisting of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166.

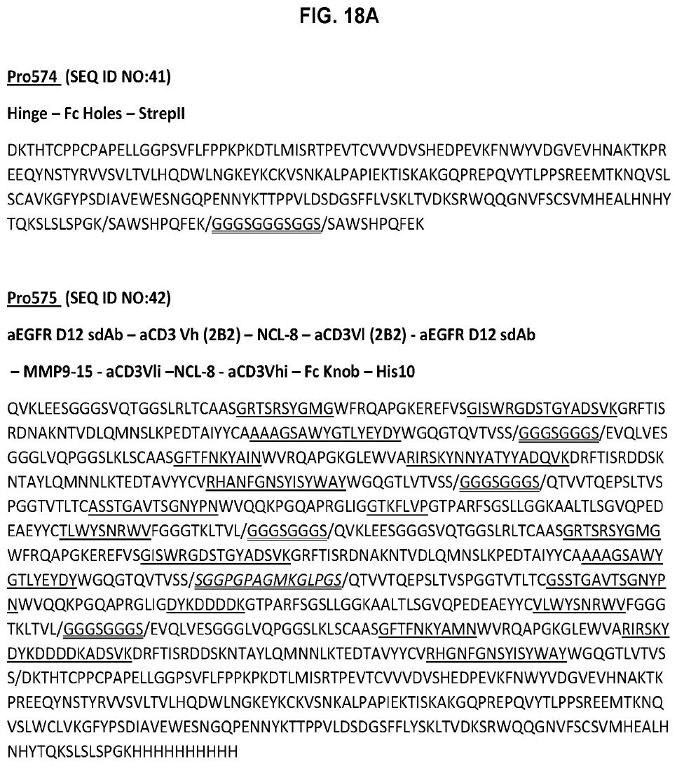

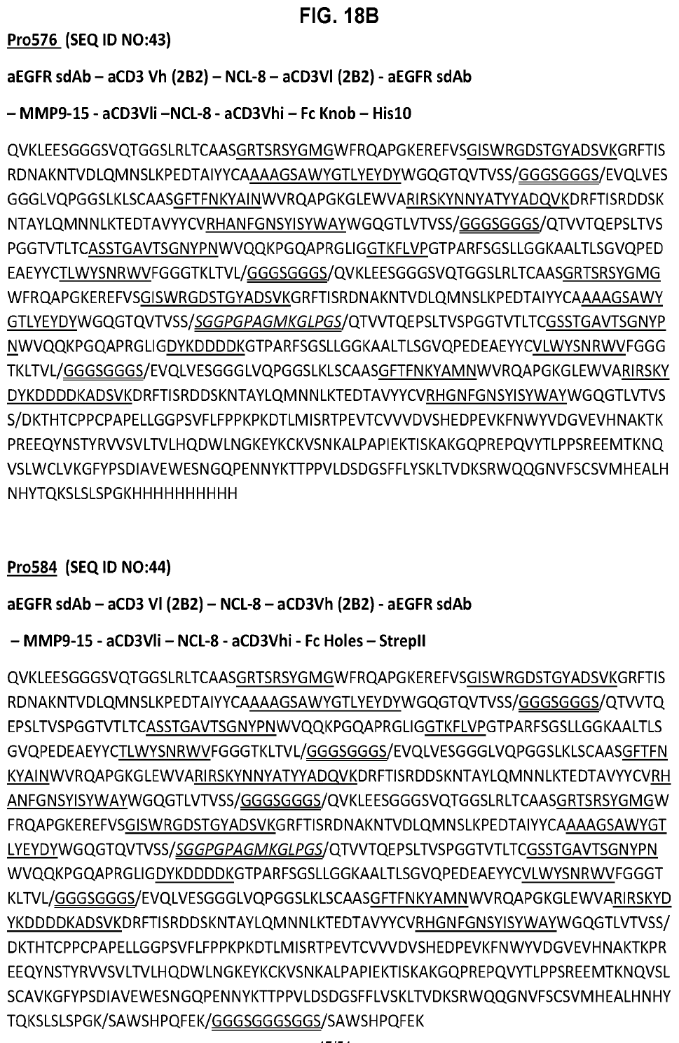

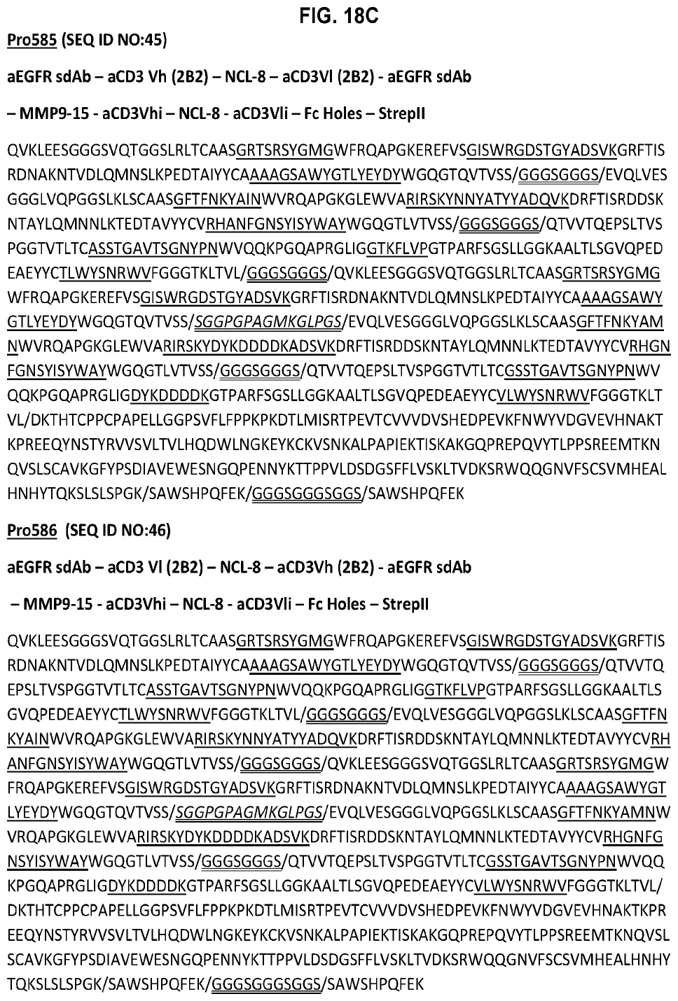

Provided herein are heterodimeric Fc fusion prodrug proteins comprising, a first monomeric Fc-hole polypeptide (see, e.g., FIG. 18A such as Pro574) comprising (from N- to C-terminal) a domain linker (hinge linker)-Fc hole domain and a second monomeric Fc-knob polypeptide (see, e.g., FIG. 18A such as Pro575) comprising (from N- to C-terminal) an ABD against a TTA-domain linker-constrained anti-CD3 Fv domain (e.g., either an active VL domain-non-cleavable constrained linker (NCCL)-active VH domain or an active VH domain-non-cleavable constrained linker (NCCL)-active VL domain)-an ABD against a TTA-cleavable linker-an anti-CD3 pseudo Fv domain (e.g., either an inactive VH domain-noncleavable linker-inactive VL domain or an inactive VL domain-noncleavable linker-inactive VH domain)-Fc knob domain. In some embodiments, the C-terminal end of the first monomeric Fc polypeptide includes a tag such as, but not limited to a (His)10 tag or a Strep-II tag. In some embodiments, the C-terminal end of the second monomeric Fc polypeptide includes a tag such as, but not limited to a (His)10 tag or a Strep-II tag. In some embodiments, the heterodimeric Fc fusion prodrug protein comprises a first monomeric Fc-hole polypeptide of Pro574. In some embodiments, the heterodimeric Fc fusion prodrug protein comprises a second monomeric Fc-knob polypeptide of Pro575 or Pro576. The amino acid sequence of Pro574 is depicted in FIG. 18A. The amino acid sequence of Pro575 is depicted in FIG. 18A. The amino acid sequence of Pro576 is depicted in FIG. 18B. The amino acid sequences of Pro575 and Pro576 are identical. In some embodiments, the nucleic acid sequence encoding Pro575 is different than the nucleic acid sequence encoding Pro576. In some embodiments, the nucleic acid sequence encoding Pro575 has at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the nucleic acid sequence encoding Pro576. In some embodiments, any of the ABDs against a TTA can be used in the heterodimeric Fc protein. In one embodiment, the sdABD is selected from the group consisting of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166.

Provided herein are heterodimeric Fc fusion prodrug proteins comprising, a first monomeric Fc-knob polypeptide (see, e.g., FIG. 18A such as Pro575 and FIG. 18B such as Pro576) comprising (from N- to C-terminal) an ABD against a TTA-domain linker-constrained anti-CD3 Fv domain (e.g., an active VH domain-non-cleavable constrained linker (NCCL)-active VL domain)-an ABD against a TTA-cleavable linker-an anti-CD3 pseudo Fv domain (e.g., an inactive VL domain-noncleavable linker-inactive VH domain)-Fc knob domain and a second monomeric Fc-hole polypeptide comprising (from N- to C-terminal) an ABD against a TTA-domain linker-constrained anti-CD3 Fv domain (e.g., either an active VL domain-non-cleavable constrained linker (NCCL)-active VH domain or an active VH domain-non-cleavable constrained linker (NCCL)-active VL domain)-an ABD against a TTA-cleavable linker-an anti-CD3 pseudo Fv domain (e.g., either an inactive VH domain-noncleavable linker-inactive VL domain or an inactive VL domain-noncleavable linker-inactive VH domain)-Fc hole domain. In some embodiments, the C-terminal end of the second monomeric Fc polypeptide includes a tag such as, but not limited to a (His)10 tag or a Strep-II tag. In some embodiments, the heterodimeric Fc fusion prodrug protein comprises a first monomeric Fc-knob polypeptide of Pro575 or Pro576. In some embodiments, the heterodimeric Fc fusion prodrug protein comprises a second monomeric Fc-hole polypeptide selected from the group consisting of Pro584, Pro585, and Pro586. The amino acid sequence of Pro575 is depicted in FIG. 18A. The amino acid sequence of Pro576 is depicted in FIG. 18B. The amino acid sequence of Pro584 is depicted in FIG. 18B. The amino acid sequence of Pro585 is depicted in FIG. 18C. The amino acid sequence of Pro586 is depicted in FIG. 18C. In some embodiments, any of the ABDs against a TTA can be used in the heterodimeric Fc protein. In one embodiment, the sdABD is selected from the group consisting of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166.

In some embodiments, the heterodimeric Fc fusion prodrug protein comprises Pro575 and Pro574. In some embodiments, the heterodimeric Fc fusion prodrug protein comprises Pro575 and Pro584. In some embodiments, the heterodimeric Fc fusion prodrug protein comprises Pro575 and Pro585. In some embodiments, the heterodimeric Fc fusion prodrug protein comprises Pro575 and Pro586. In some embodiments, the heterodimeric Fc fusion prodrug protein comprises Pro576 and Pro574. In some embodiments, the heterodimeric Fc fusion prodrug protein comprises Pro576 and Pro584. In some embodiments, the heterodimeric Fc fusion prodrug protein comprises Pro576 and Pro585. In some embodiments, the heterodimeric Fc fusion prodrug protein comprises Pro576 and Pro586.

### VI. Additional Embodiments of the Invention

In one aspect, provided herein is a heterodimeric protein composition comprising: (a) a first monomer comprising, from N- to C-terminal: (i) a first antigen binding domain that binds to a first tumor target antigen (TTA); (ii) a domain linker; (iii) a constrained Fv domain comprising: (1) a variable heavy domain comprising vhCDR1, vhCDR2, and vhCDR3; (2) a constrained non-cleavable linker; and (3) a variable light domain comprising vlCDR1, vlCDR2, and vlCDR3; (iv) a first cleavable linker; and (v) a first Fc domain; and (b) a second monomer comprising, from N- to C terminal: (i) a pseudo Fv domain comprising: (1) a pseudo variable heavy domain; (2) a non-cleavable linker; and (3) a pseudo variable light domain; (ii) a second cleavable linker; and (iii) a second Fc domain, wherein the first Fc domain and second Fc domain comprise a knob-in-hole modification, and wherein the constrained Fv domain does not bind human CD3 in the absence of cleavage at the cleavable linkers. In some embodiments, the first monomer further comprises a second antigen binding domain that binds to a second tumor target antigen (TTA) at the N-terminus of the first cleavable linker.

In certain embodiments, the variable heavy chain comprises the amino acid sequence of SEQ ID NO:186. In some instances, the variable heavy domain comprises vhFR1-vhCDR1-vhFR2-vhCDR2-vhFR3-vhCDR3-vhFR4.

In some embodiments, the variable light domain comprises the amino acid sequence of SEQ ID NO:170. In certain instances, the variable light domain comprises vlFR1-vlCDR1-vlFR2-vlCDR2-vlFR3-vlCDR3-vlFR4.

In certain embodiments, the pseudo heavy domain comprises the amino acid sequence of SEQ ID NO:190. In some embodiments, the pseudo light domain comprises the amino acid sequence of SEQ ID NO:174. In certain embodiments, the pseudo heavy domain comprises the amino acid sequence of SEQ ID NO:194. In some embodiments, the pseudo light domain comprises the amino acid sequence of SEQ ID NO:178. In certain embodiments, the pseudo heavy domain comprises the amino acid sequence of SEQ ID NO:198. In some embodiments, the pseudo light domain comprises the amino acid sequence of SEQ ID NO:182.

In some embodiments, the first TTA is selected from the group consisting of EGFR, EpCAM, FOLR1, B7H3, Trop2, and CA9. In other embodiments, the second TTA is selected from the group consisting of EGFR, EpCAM, FOLR1, B7H3, Trop2, and CA9. In particular instances, the first TTA and the second TTA are the same. In certain instances, the first TTA and the second TTA are different.

In some embodiments, the first antigen binding domain comprises the amino acid sequence of any one of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166. In other embodiments, the second antigen binding domain comprises the amino acid sequence of any one of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166.

In some embodiments, the first and/or second cleavable linker contains a cleavage site for MMP9. In some embodiments, the first and/or second cleavable linker contains a cleavage site for meprin.

In various embodiments, the heterodimeric protein comprises the amino acid sequences of Pro217 and Pro218, Pro219 and Pro218, SEQ ID NOS:9 and 10, or SEQ ID NOS:10 and 11.

In another aspect, provided herein is a heterodimeric protein composition comprising: (a) a first monomer comprising, from N- to C-terminal: (i) a first antigen binding domain that binds to a first tumor target antigen (TTA); (ii) a first domain linker; (iii) a first pseudo Fv domain comprising: (1) a variable light domain comprising vlCDR1, vlCDR2, and vlCDR3; (2) a first cleavable linker; and (3) a pseudo variable heavy domain; and (iv) a first Fc domain; and (b) a second monomer comprising, from N- to C terminal: (i) a second antigen binding domain that binds to a second tumor target antigen (TTA); (ii) a second domain linker; (iii) a second pseudo Fv domain comprising: (1) a variable heavy domain comprising vhCDR1, vhCDR2, and vhCDR3; (2) a second cleavable linker; and (3) a pseudo variable light domain; and (iv) a first Fc domain; and wherein the first Fc domain and second Fc domain comprise a knob-in-hole modification, and wherein the variable light domain of the first pseudo Fv domains and the variable heavy domain of second pseudo Fv domains do not bind human CD3 in the absence of cleavage at the cleavable linkers.

In certain embodiments, the first monomer further comprises a first hinge linker at the N-terminus of the first Fc domain. In some embodiments, the second monomer further comprises a second hinge linker at the N-terminus of the second Fc domain. In various embodiments, the first monomer comprises a first hinge linker at the N-terminus of the first Fc domain and the second monomer comprises a second hinge linker at the N-terminus of the second Fc domain.

In certain embodiments, the variable heavy chain comprises the amino acid sequence of SEQ ID NO:186. In some instances, the variable heavy domain comprises vhFR1-vhCDR1-vhFR2-vhCDR2-vhFR3-vhCDR3-vhFR4.

In some embodiments, the variable light domain comprises the amino acid sequence of SEQ ID NO:170. In some instances, the variable light domain comprises vlFR1-vlCDR1-vlFR2-vlCDR2-vlFR3-vlCDR3-vlFR4.

In certain embodiments, the pseudo heavy domain comprises the amino acid sequence of SEQ ID NO:190. In some embodiments, the pseudo light domain comprises the amino acid sequence of SEQ ID NO:174. In certain embodiments, the pseudo heavy domain comprises the amino acid sequence of SEQ ID NO:194. In some embodiments, the pseudo light domain comprises the amino acid sequence of SEQ ID NO:178. In certain embodiments, the pseudo heavy domain comprises the amino acid sequence of SEQ ID NO:198. In some embodiments, the pseudo light domain comprises the amino acid sequence of SEQ ID NO:182.

In some embodiments, the first TTA is selected from the group consisting of EGFR, EpCAM, FOLR1, B7H3, Trop2, and CA9. In other embodiments, the second TTA is selected from the group consisting of EGFR, EpCAM, FOLR1, B7H3, Trop2, and CA9. In particular instances, the first TTA and the second TTA are the same. In certain instances, the first TTA and the second TTA are different.

In some embodiments, the first antigen binding domain comprises the amino acid sequence of any one of SEQ ID NOS:50, 54, 58, 62, 66, 70, 74, 78, 82, 86, 90, 94, 98, 102, 106, 110, 114, 118, 122, 126, 130, 134, 138, M2, 146, 150, 154, 158, 162, and 166. In other embodiments, the second antigen binding domain comprises the amino acid sequence of any one of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166.

In some embodiments, the first and/or second cleavable linker contains a cleavage site for MMP9. In some embodiments, the first and/or second cleavable linker contains a cleavage site for meprin.

In some embodiments, the heterodimeric protein comprises the amino acid sequences of Pro36 and Pro37, Pro36 and Pro38, Pro67 and Pro68, SEQ ID NOS:1 and 2, SEQ ID NOS:1 and 3, or SEQ ID NOS: 4 and 5.

In yet another aspect of the invention, provided herein is a heterodimeric protein composition comprising: (a) a first monomer comprising, from N- to C-terminal: (i) a first antigen binding domain that binds to a first tumor target antigen (TTA); and (ii) a first Fc domain; and (b) a second monomer comprising, from N- to C-terminal: (i) a second antigen binding domain that binds to a second tumor target antigen (TTA); (ii) a domain linker; (iii) a first pseudo Fv domain comprising: (1) a variable heavy domain comprising vhCDR1, vhCDR2, and vhCDR3, (2) a first cleavable linker; and (3) a pseudo variable light domain; (iv) a second Fc domain; (v) a second cleavable linker; (vi) a third antigen binding domain that binds to a third tumor target antigen (TTA); and (vii) a second pseudo Fv domain comprising: (1) a variable light domain comprising vlCDR1, vlCDR2, and vlCDR3; (2) a third cleavable linker; and (3) a pseudo variable heavy domain; wherein the first Fc domain and second Fc domain comprise a knob-in-hole modification, and the variable heavy domain of the first pseudo Fv domain and the variable light domain of the second pseudo Fv domain do not bind human CD3 in the absence of cleavage at the cleavable linkers.

In certain embodiments, the first monomer further comprises a first hinge linker at the N-terminus of the first Fc domain. In some embodiments, the second monomer further comprises a second hinge linker at the N-terminus of the second Fc domain. In various embodiments, the first monomer comprises a first hinge linker at the N-terminus of the first Fc domain and the second monomer comprises a second hinge linker at the N-terminus of the second Fc domain.

In certain embodiments, the variable heavy chain comprises the amino acid sequence of SEQ ID NO:186. In some instances, the variable heavy domain comprises vhFR1-vhCDR1-vhFR2-vhCDR2-vhFR3-vhCDR3-vhFR4

In some embodiments, the variable light domain comprises the amino acid sequence of SEQ ID NO:170. In various instances, the variable light domain comprises vlFR1-vlCDR1-vlFR2-vlCDR2-vlFR3-vlCDR3-vlFR4.

In certain embodiments, the pseudo heavy domain comprises the amino acid sequence of SEQ ID NO:190. In some embodiments, the pseudo light domain comprises the amino acid sequence of SEQ ID NO:174. In certain embodiments, the pseudo heavy domain comprises the amino acid sequence of SEQ ID NO:194. In some embodiments, the pseudo light domain comprises the amino acid sequence of SEQ ID NO:178. In certain embodiments, the pseudo heavy domain comprises the amino acid sequence of SEQ ID NO:198. In some embodiments, the pseudo light domain comprises the amino acid sequence of SEQ ID NO:182.

In some embodiments, the first TTA is selected from the group consisting of EGFR, EpCAM, FOLR1, B7H3, Trop2, and CA9. In other embodiments, the second TTA is selected from the group consisting of EGFR, EpCAM, FOLR1, B7H3, Trop2, and CA9. In particular instances, the first TTA and the second TTA are the same. In certain instances, the first TTA and the second TTA are different.

In some embodiments, the first TTA, second TTA, and/or third TTA is selected from the group consisting of EGFR, EpCAM, FOLR1, B7H3, Trop2, and CA9. In some cases, the first TTA is selected from EGFR, EpCAM, FOLR1, B7H3, Trop2, and CA9. In other cases, the first TTA is EpCAM. In some cases, the second TTA is selected from EGFR, EpCAM, FOLR1, B7H3, Trop2, and CA9. In other cases, the second TTA is EpCAM. In certain cases, the third TTA is selected from EGFR, EpCAM, FOLR1, B7H3, Trop2, and CA9.

In certain embodiments, the first TTA and the second TTA, or the first TTA and the third TTA, or the second TTA and the third TTA, or the first TTA, the second TTA, and the third TTA are the same. In particular embodiments, the first TTA and the second TTA, or the first TTA and the third TTA, or the second TTA and the third TTA, or the first TTA, the second TTA, and the third TTA are different.

In various embodiments, the first antigen binding domain comprises the amino acid sequence of any one of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166. In some embodiments, the second antigen binding domain comprises the amino acid sequence of any one of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166. In various embodiments, the third antigen binding domain comprises the amino acid sequence of any one of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166.

In some embodiments, the first, second and/or third cleavable linker contains a cleavage site for MMP9. In other embodiments, the first, second and/or third cleavable linker contains a cleavage site for meprin.

In some embodiments, the heterodimeric protein comprises the amino acid sequences of Pro69 and Pro70, or SEQ ID NOS:6 and 7.

In another aspect, provided herein is a heterodimeric protein composition comprising: (a) a first monomer comprising, from N- to C-terminal: (i) a first antigen binding domain that binds to a first tumor target antigen (TTA); (ii) a first Fc domain; (iii) a first cleavable linker; (iv) a second antigen binding domain that binds to a second tumor target antigen (TTA); (v) a first domain linker; and (vi) a first pseudo Fv domain comprising: (1) a variable light domain comprising vlCDR1, vlCDR2, and vlCDR3; (2) a second cleavable linker; (3) a pseudo variable heavy domain; and (b) a second monomer comprising, from N- to C-terminal: (i) a second antigen binding domain that binds to a second tumor target antigen (TTA); (ii) a second domain linker; (iii) a second pseudo Fv domain comprising: (1) a variable heavy domain comprising vhCDR1, vhCDR2, and vhCDR3; (2) a third cleavable linker; and (3) a pseudo variable light domain; and (iv) a second Fc domain; wherein the first Fc domain and second Fc domain comprise a knob-in-hole modification, and wherein the variable light domain of the first pseudo Fv domain and the variable heavy domain of the second pseudo Fv domain do not bind human CD3 in the absence of cleavage at the cleavable linkers.

In some embodiments, the first monomer further comprises a first hinge linker at the N-terminus of the first Fc domain. In some embodiments, the second monomer further comprises a second hinge linker at the N-terminus of the second Fc domain. In various embodiments, the first monomer comprises a first hinge linker at the N-terminus of the first Fc domain and the second monomer comprises a second hinge linker at the N-terminus of the second Fc domain.

In certain embodiments, the variable heavy chain comprises the amino acid sequence of SEQ ID NO:186. In some instances, the variable heavy domain comprises vhFR1-vhCDR1-vhFR2-vhCDR2-vhFR3-vhCDR3-vhFR4.

In some embodiments, the variable light domain comprises the amino acid sequence of SEQ ID NO:170. In various instances, the variable light domain comprises vlFR1-vlCDR1-vlFR2-vlCDR2-vlFR3-vlCDR3-vlFR4.

In some embodiments, the first antigen binding domain comprises the amino acid sequence of any one of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166. In other embodiments, the second antigen binding domain comprises the amino acid sequence of any one of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166.

In some embodiments, the first TTA is selected from the group consisting of EGFR, EpCAM, FOLR1, B7H3, Trop2, and CA9. In other embodiments, the second TTA is selected from the group consisting of EGFR, EpCAM, FOLR1, B7H3, Trop2, and CA9. In particular instances, the first TTA and the second TTA are the same. In certain instances, the first TTA and the second TTA are different.

In some embodiments, the first and/or second cleavable linker contains a cleavage site for MMP9. In some embodiments, the first and/or second cleavable linker contains a cleavage site for meprin.

In some embodiments, the heterodimeric protein comprises the amino acid sequences of Pro67 and Pro71 or SEQ ID NOS:4 and 8.

Provided herein is a nucleic acid encoding the first monomer of any one of the heterodimeric proteins described herein. Also provided is a nucleic acid encoding the second monomer of any one of the heterodimeric proteins described herein. In addition, provided herein is an expression vector comprising the nucleic acid encoding the first monomer, an expression vector comprising the nucleic acid encoding the second monomer, or an expression vector comprising the nucleic acid encoding the first monomer and the nucleic acid encoding the second monomer. In some embodiments, provided herein is a host cell comprising any one of the expression vectors disclosed herein.

In one aspect of the invention, provided is a method of making any one of the heterodimeric proteins described herein. The method comprises culturing the host cell described herein under conditions to express the heterodimeric protein and recovering the heterodimeric protein.

In one aspect of the invention, provided is a method of treating cancer comprising administering any one of the heterodimeric proteins of the present invention to a patient.

The heterodimeric protein composition described herein can be referred to as a prodrug composition in the absence of cleavage by a cognate protease.

In another aspect, provided herein is a method of treating cancer in a human subject in need thereof comprising administering any prodrug composition described herein.

### VII. Examples

### A. Example 1: Pro Construct Construction and Purification

Transfections

Each pair of constructs were expressed from a separate expression vector (pcdna3.4 derivative). Equal amounts of plasmid DNA that encoded the pair of hemi-COBRAs were mixed and transfected to Expi293 cells following the manufacture's transfection protocol. Conditioned media was harvested 5 days post transfection by centrifugation (6000 rpm×25′) and filtration (0.2 uM filter). Protein expression was confirmed by SDS-PAGE. Constructs were purified and The final buffer composition was: 25 mM Citrate, 75 mM Arginine, 75 mM NaCl, 4% Sucrose, pH 7. The final preparations were stored at −80° C.

Protease Cleavages

EK

Recombinant human enterokinease (R&D Systems Cat. No. 1585-SE-010) was used to cleave the heterodimeric Fc prodrug proteins described herein. Recombinant proteases were activated according to manufacturer procedures and prepared at stock concentration of about 100 mM.

Test samples (Pro36+37, Pro36+38, Pro67+68, Pro69+70, Pro67+71, Pro217+218, and Pro218+219) were buffered exchanged into HEPES Buffered Saline with calcium chloride (25 mM HEPES, 50 mM NaCl, 2 mM CaCl₂) and incubated with the appropriate protease at 10 nM final concentration overnight at room temperature. The cleavage was confirmed by SDS-PAGE.

MMP-9

Activation of MMP9:

Recombinant human MMP9 was activated according to the following protocol. Recombinant human MMP-9 (R&D #911-MP-010) is at 0.44 mg/ml (4.7 uM). p-aminophenylmercuric acetate (APMA) (Sigma) is prepared at the stock concentration of 100 mM in DMSO. Assay buffer was 50 mM Tris pH 7.5, 10 mM CaCl₂), 150 mM NaCl, 0.05% Brij-35.
Dilute rhMMP9 with assay buffer to ˜100 ug/ml (25 ul hMMP9+75 uL assay buffer)
Add p-aminophenylmercuric acetate (APMA) from 100 mM stock in DMSO to a final concentration of 1 mM (1 uL to 100 uL)
Incubate at 37′C for 24 hrs
Dilute MMP9 to 10 ng/ul (add 900 ul of assay buffer to 100 ul of activated solution)

The concentration of the activated rhMMP9 is ˜100 nM.

Cleavage of Constructs for TDCC Assays

To cleave the constructs, 100 ul of the protein sample at 1 mg/ml concentration (10.5 uM) in the formulation buffer (25 mM Citric acid, 75 mM L-arginine, 75 mM NaCl, 4% sucrose) was supplied with CaCl2 up to 10 mM. Activated rhMMP9 was added to the concentration 20-35 nM. The sample was incubated at room temperature overnight (16-20 hrs). The completeness of cleavage was verified using SDS PAGE (10-20% TG, TG running buffer, 200v, 1 hr). Samples were typically 98% cleaved.

### B. Example 2: T-Cell Dependent Cellular Cytotoxicity (TDCC) Assay to Test Potency of Activated Heterodimeric Fc Prodrug Proteins

Firefly Luciferase transduced HT-29 cells were grown to approximately 80% confluency and detached with Versene (0.48 mM EDTA in PBS—Ca—Mg). Cells were centrifuged and resuspended in TDCC media (5% Heat Inactivated FBS in RPMI 1640 with HEPES, GlutaMax, Sodium Pyruvate, Non-essential amino acids, and β-mercaptoethanol). Purified human Pan-T cells were thawed, centrifuged and resuspended in TDCC media.

A coculture of HT-29_Luc cells and T cells was added to 384-well cell culture plates. Serially diluted COBRAs were then added to the coculture and incubated at 37 EC for 48 hours. Finally, an equal volume of SteadyGlo luciferase assay reagent was added to the plates and incubated for 20 minutes. The plates were read on the Perkin Elmer Envision with an exposure time of 0.1s/well. Total luminescence was recorded and data were analyzed on GraphPad Prism 7.

We tested the percentage of specific cytotoxicity induced when an activated (cleaved) heterodimeric Fc prodrug protein engages with T cells and directs cytolysis toward target positive tumor cells using the assay above.

FIG. 3A and FIG. 3B show that some illustrative heterodimeric Fc prodrug constructs such as Pro36+37 and Pro36+38 displayed low or a lack of conditionality upon cleavage with a cognate protease in a TDCC assay.

FIG. 7A, FIG. 7B, and FIG. 7C show that some illustrative heterodimeric Fc prodrug constructs such as Pro67+68 and Pro69+70 displayed conditionality but lacked high activity when cleaved with a cognate protease in a TDCC assay.

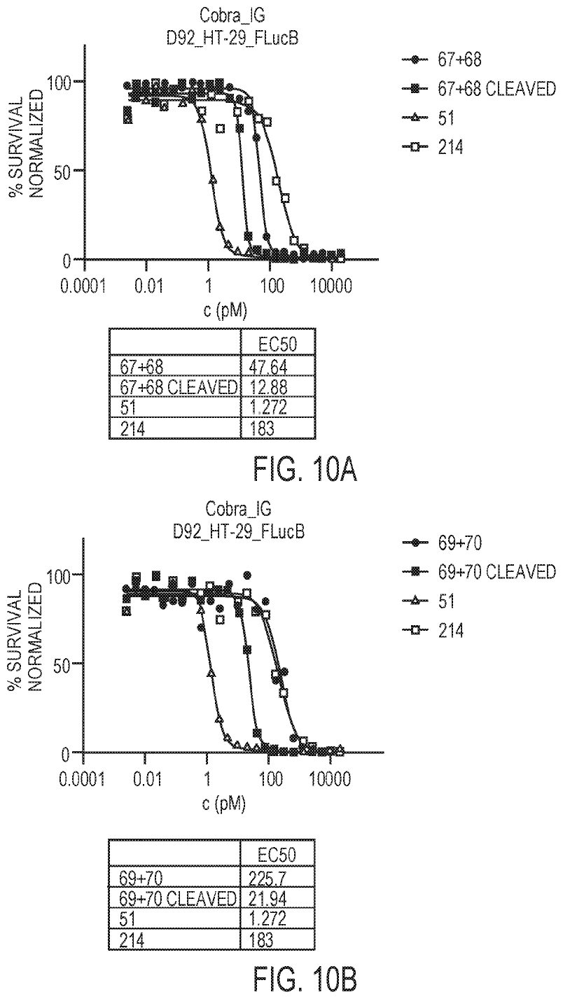

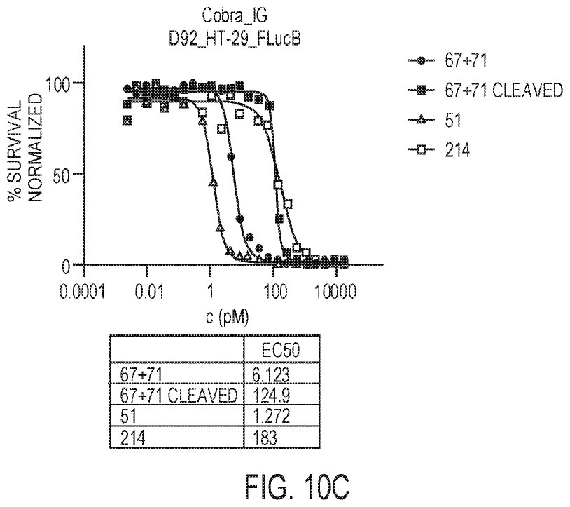

FIG. 10A and FIG. 10B show that illustrative heterodimeric Fc prodrug constructs such as Pro217+218 and Pro218+219 displayed conditionality and high potency when cleaved with a cognate protease in a TDCC assay.

The prodrug constructs described herein containing a single domain antibody against EGFR induced cancer cell killing upon protease cleavage in a comparable manner to a fusion protein comprises a single domain antibody against EGFR and an anti-CD3 Fv domain.

### C. Example 3: Adoptive T-Cell Transfer Model

NSG mice (Jackson) were implanted with tumor cell lines subcutaneously. Human T-cells were isolated from leukopak via negative selection (StemCell Technologies) and expanded utilizing G-Rex technology (Wilson Wolf) utilizing T-cell expansion/activation beads (Miltenyi). Once tumor growth was established, mice were randomized based on tumor volume, expanded human T-cells were implanted i.v. and test articles were dosed as indicated. Tumor volume was assessed by caliper measurement.

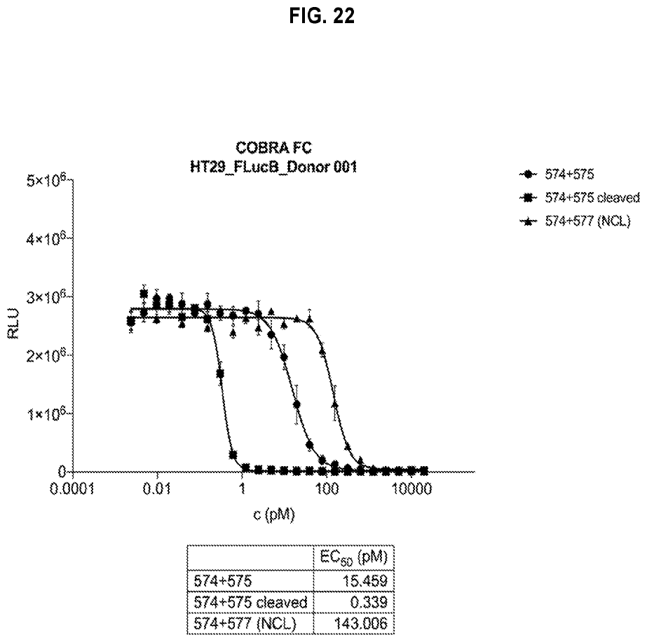

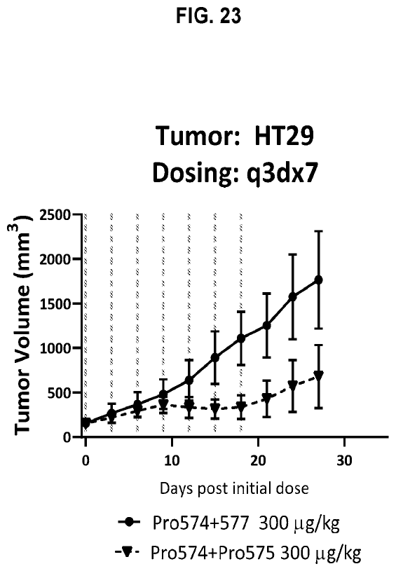

The test articles used were Pro574 and Pro575, and Pro574 and Pro577 (see, e.g., FIG. 21). The COBRA knob-hole Fc proteins were potent as shown in FIG. 22. Pro574 (empty hole) when combined with MMP9 cleaved knob Pro 575 was potent, compared to Pro574 combined with Pro577 (NCL knob control).

Administration of the heterodimeric Fc protein of Pro574/Pro575 demonstrated an anti-tumor response compared to Pro574/577 (NCL Control) the adoptive T cell transfer model.

All cited references are herein expressly incorporated by reference in their entirety. Whereas particular embodiments of the invention have been described above for purposes of illustration, it will be appreciated by those skilled in the art that numerous variations of the details may be made without departing from the invention as described in the appended claims.

What is claimed is:

1. A homodimeric protein composition comprising:
(a) two monomers each comprising, from N- to C-terminal:
i) a first single domain antigen binding domain (sdABD) that binds to a first tumor target antigen (TTA) (sdABD-TTA);
ii) an optional domain linker;
iii) a constrained Fv domain comprising:
1) a variable heavy domain comprising vhCDR1, vhCDR2, and vhCDR3;
2) a constrained, non-cleavable linker (CNCL); and
3) a variable light domain comprising vlCDR1, vlCDR2, and vlCDR3;
iv) an optional domain linker;
v) a second sdABD-TTA;
vi) a cleavable linker;
vii) a pseudo Fv domain comprising:
1) a pseudo variable light domain;
2) a non-cleavable linker; and
3) a pseudo variable heavy domain; and
viii) an optional cleavable linker; and
ix) an Fc domain;
wherein said variable heavy domain and first variable light domain are capable of binding human CD3 but said constrained Fv domain does not bind CD3; wherein said variable heavy domain and said pseudo variable light domain intermolecularly associate to form an inactive Fv; and wherein said variable light domain and said pseudo variable heavy domain intermolecularly associate to form an inactive Fv.

2. The homodimeric protein composition according to claim 1, wherein said first variable heavy domain is N-terminal to said first variable light domain and said pseudo variable light domain is N-terminal to said pseudo variable heavy domain.

3. The homodimeric protein composition according to claim 1, wherein said first variable light domain is N-terminal to said first variable heavy domain and said pseudo variable light domain is N-terminal to said pseudo variable heavy domain.

4. The homodimeric protein composition according to claim 1, wherein said first variable light domain is N-terminal to said first variable heavy domain and said pseudo variable heavy domain is N-terminal to said pseudo variable light domain.

5. The homodimeric protein composition according to claim 1, wherein said first variable heavy domain is N-terminal to said first variable light domain and said pseudo variable heavy domain is N-terminal to said pseudo variable light domain.

6. The homodimeric protein composition according to any of claims 1 to 5, wherein the variable heavy chain comprises the amino acid sequence of SEQ ID NO:186 and the variable light domain comprises the amino acid sequence of SEQ ID NO:170.

7. The homodimeric protein composition of any of claims 1 to 6, wherein the pseudo variable heavy domain comprises the amino acid sequence of SEQ ID NO:190 and the pseudo variable light domain comprises the amino acid sequence of SEQ ID NO:174.

8. The homodimeric protein composition of any of claims 1 to 6, wherein the pseudo variable heavy domain comprises the amino acid sequence of SEQ ID NO:194 and the pseudo variable light domain comprises the amino acid sequence of SEQ ID NO:178.

9. The homodimeric protein composition of any of claims 1 to 6, wherein the pseudo variable heavy domain comprises the amino acid sequence of SEQ ID NO:198 and the pseudo variable light domain comprises the amino acid sequence of SEQ ID NO:182.

10. The homodimeric protein composition of any of claims 1 to 9, wherein the TTA is selected from the group consisting of EGFR, FOLR1, B7H3, EpCAM, Trop2, and CA9.

11. The homodimeric protein composition of any of claims 1 to 10, wherein said first and second sdABDs bind to the same TTA.

12. The homodimeric protein composition according to any of claims 1 to 10, wherein said first and second sdABDs bind to different TTAs.

13. The homodimeric protein composition according to any of claims 1 to 11, wherein said first and second sdABD-TTAs are the same.

14. The homodimeric protein composition according to any of claims 1 to 12, wherein said first and second sdABD-TTAs are different.

15. The homodimeric protein composition of any of claims 1 to 14, wherein said sdABD(s) is selected from the group consisting of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166.

16. The homodimeric protein composition of any of claims 1 to 15, wherein said first cleavable linker and/or said optional cleavable linker are cleaved by a human protease selected from the group consisting of MMP2, MMP9, Cathepsin S, Cathepsin K, Cathespin L, GranzymeB, uPA, Kallekriein7, matriptase and thrombin.

17. The homodimeric protein composition of any of claims 1 to 16 wherein each monomer comprises an amino acid sequence selected from the group consisting of Pro556 (SEQ ID NO:36), Pro587 (SEQ ID NO:38), Pro588 (SEQ ID NO:39), and Pro589 (SEQ ID NO:40).

18. A nucleic acid composition comprising a nucleic acid encoding said monomer of any of claims 1 to 17.

19. An expression vector composition comprising said nucleic acid according to claim 18.

20. A host cell comprising said expression vector composition according to claim 19.

21. A method of making a homodimeric protein of any of claims 1 to 17 comprising: culturing the host cell of claim 20 under conditions to express the homodimeric protein, and recovering the heterodimeric protein.

22. A method of treating cancer comprising administering the homodimeric protein of any one of claims 1 to 17.

23. A heterodimeric protein composition comprising:
(a) a first Fc monomer comprising a first Fc domain; and
(b) a second Fc monomer comprising, from N- to C terminal:
i) a first single domain antigen binding domain (sdABD) that binds to a first tumor target antigen (TTA) (sdABD-TTA);
ii) an optional domain linker;
iii) a constrained Fv domain comprising:
1) a variable heavy domain comprising vhCDR1, vhCDR2, and vhCDR3;
2) a constrained, non-cleavable linker (CNCL); and
3) a variable light domain comprising vlCDR1, vlCDR2, and vlCDR3;
iv) an optional domain linker;
v) a second sdABD-TTA;
vi) a first cleavable linker;
vii) a pseudo Fv domain comprising:
1) a pseudo variable light domain;
2) a non-cleavable linker; and
3) a pseudo variable heavy domain; and
viii) an optional second cleavable linker; and
ix) a second Fc domain;
wherein said first Fc domain and said second Fc domain comprise a knob-in hole modification; wherein said variable heavy domain and said variable light domain are capable of binding human CD3 but said constrained Fv domains do not bind CD3; wherein said variable heavy domain and said pseudo variable light domain intermolecularly associate to form an inactive Fv; and wherein said variable light domain and said pseudo variable heavy domain intermolecularly associate to form an inactive Fv.

24. The heterodimeric protein composition according to claim 23, wherein said variable heavy domain is N-terminal to said variable light domain and said pseudo variable light domain is N-terminal to said pseudo variable heavy domain.

25. The heterodimeric protein composition according to claim 23, wherein said variable heavy domain is N-terminal to said variable light domain and said pseudo variable heavy domain is N-terminal to said pseudo variable light domain.

26. The heterodimeric protein composition according to claim 23, wherein said variable light domain is N-terminal to said variable heavy domain and said pseudo variable light domain is N-terminal to said pseudo variable heavy domain.

27. The heterodimeric protein composition according to claim 23, wherein said variable light domain is N-terminal to said variable heavy domain and said pseudo variable heavy domain is N-terminal to said pseudo variable light domain.

28. The heterodimeric protein composition according to any of claims 23 to 27, wherein said first Fc domain comprises a Fc-hole domain and said second Fc domain comprises a Fc-knob domain.

29. The heterodimeric protein composition according to any of claims 23 to 28, wherein the variable heavy chain comprises the amino acid sequence of SEQ ID NO:186 and the variable light domain comprises the amino acid sequence of SEQ ID NO:170.

30. The heterodimeric protein composition of any of claims 23 to 29, wherein the pseudo variable heavy domain comprises the amino acid sequence of SEQ ID NO:190 and the pseudo variable light domain comprises the amino acid sequence of SEQ ID NO:174.

31. The heterodimeric protein composition of any of claims 23 to 29, wherein the pseudo variable heavy domain comprises the amino acid sequence of SEQ ID NO:194 and the pseudo variable light domain comprises the amino acid sequence of SEQ ID NO:178.

32. The heterodimeric protein composition of any of claims 23 to 29, wherein the pseudo variable heavy domain comprises the amino acid sequence of SEQ ID NO:198 and the pseudo variable light domain comprises the amino acid sequence of SEQ ID NO:182.

33. The heterodimeric protein composition of any of claims 23 to 32, wherein the TTA is selected from the group consisting of EGFR, FOLR1, B7H3, EpCAM, Trop2, and CA9.

34. The heterodimeric protein composition of any of claims 23 to 33, wherein said first and second sdABDs bind to the same TTA.

35. The heterodimeric protein composition according to any of claims 23 to 33, wherein said first and second sdABDs bind to different TTAs.

36. The heterodimeric protein composition according to any of claims 23 to 34, wherein said first and second sdABD-TTAs are the same.

37. The heterodimeric protein composition according to any of claims 23 to 35, wherein said first and second sdABD-TTAs are different.

38. The heterodimeric protein composition of any of claims 23 to 37, wherein said sdABD(s) is selected from the group consisting of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166.

39. The heterodimeric protein composition of any of claims 23 to 38, wherein said cleavable linker and/or said optional cleavable linker are cleaved by a human protease selected from the group consisting of MMP2, MMP9, Cathepsin S, Cathepsin K, Cathespin L, GranzymeB, uPA, Kallekriein7, matriptase, and thrombin.

40. The heterodimeric protein composition of any of claims 23 to 39, wherein said first Fc monomer comprises an amino acid sequence selected from the group consisting of Pro574 (SEQ ID NO:41) and Pro688 (SEQ ID NO:47).

41. The heterodimeric protein composition of any of claims 23 to 40, wherein said second Fc monomer comprises an amino acid sequence selected from the group consisting of Pro575 (SEQ ID NO:42), Pro576 (SEQ ID NO:43), and Pro689 (SEQ ID NO:48).

42. The heterodimeric protein composition of any of claims 23 to 41, comprising any one of the heterodimeric protein pairs selected from the group consisting of Pro574+Pro575, Pro574+Pro576, Pro688+Pro689, Pro574+Pro689, Pro688+Pro575, and Pro688+Pro576.

43. A nucleic acid composition comprising (a) a first nucleic acid encoding said first Fc monomer of any of claims 23 to 42, and/or (b) a second nucleic acid encoding said second Fc monomer of any of claims 23 to 42.

44. An expression vector composition comprising said first nucleic acid according to claim 43, and/or said second nucleic acid according to claim 43.

45. A host cell for expressing said heterodimeric protein composition of any of claims 23 to 41 comprising said expression vector composition according to claim 44.

46. A method of making a heterodimeric protein comprising: culturing the host cell of claim 45 under conditions to express the heterodimeric protein, and recovering the heterodimeric protein.

47. A method of treating cancer comprising administering the heterodimeric protein of any one of claims 23 to 42.

48. A heterodimeric protein composition comprising:
(a) a first Fc monomer comprising, from N- to C-terminal:
i) a first single domain antigen binding domain (sdABD) that binds to a first tumor target antigen (TTA) (sdABD-TTA);
ii) an optional domain linker;
iii) a first constrained Fv domain comprising:
1) a first variable heavy domain comprising vhCDR1, vhCDR2, and vhCDR3;
2) a first constrained, non-cleavable linker (CNCL); and
3) a first variable light domain comprising vlCDR1, vlCDR2, and vlCDR3;
iv) an optional domain linker;
v) a second sdABD-TTA;
vi) a first cleavable linker;
vii) a first pseudo Fv domain comprising:
1) a first pseudo variable light domain;
2) a non-cleavable linker; and
3) a first pseudo variable heavy domain;
viii) a first optional cleavable linker; and
ix) a first Fc-hole domain; and
(b) a second Fc monomer comprising, from N- to C terminal:
i) a third sdABD-TTA;
ii) an optional domain linker;
iii) a second constrained Fv domain comprising:
1) a second variable heavy domain comprising vhCDR1, vhCDR2, and vhCDR3;
2) a second CNCL; and
3) a second variable light domain comprising vlCDR1, vlCDR2, and vlCDR3;
iv) an optional domain linker;
v) a fourth sdABD-TTA;
vi) a second cleavable linker;
vii) a second pseudo Fv domain comprising:
1) a second pseudo variable light domain;
2) a non-cleavable linker; and
3) a second pseudo variable heavy domain;
viii) a second optional cleavable linker; and
ix) a second Fc-knob domain;
wherein said first variable heavy domain and said first variable light domain and said second variable heavy domain and said second variable light domain are capable of binding human CD3 but said constrained Fv domains do not bind CD3; wherein said variable heavy domains and said pseudo variable light domains intermolecularly associate to form inactive Fvs; and wherein said variable light domains and said pseudo variable heavy domains intermolecularly associate to form inactive Fvs.

49. The heterodimeric protein composition according to claim 48, wherein said first variable heavy domain is N-terminal to said first variable light domain and said first pseudo variable light domain is N-terminal to said first pseudo variable heavy domain and/or wherein said second variable heavy domain is N-terminal to said second variable light domain and said second pseudo variable light domain is N-terminal to said second pseudo variable heavy domain.

50. The heterodimeric protein composition according to claim 48, wherein said first variable light domain is N-terminal to said first variable heavy domain and said first pseudo variable light domain is N-terminal to said first pseudo variable heavy domain and/or wherein said second variable light domain is N-terminal to said second variable heavy domain and said second pseudo variable light domain is N-terminal to second first pseudo variable heavy domain.

51. The heterodimeric protein composition according to claim 48, wherein said first variable heavy domain is N-terminal to said first variable light domain and said first pseudo variable heavy domain is N-terminal to said first pseudo variable light domain and/or wherein said second variable heavy domain is N-terminal to said second variable light domain and said second pseudo variable heavy domain is N-terminal to said second pseudo variable light domain.

52. The heterodimeric protein composition according to claim 48, wherein said first variable light domain is N-terminal to said first variable heavy domain and said first pseudo variable heavy domain is N-terminal to said first pseudo variable light domain and/or wherein said second variable light domain is N-terminal to said second variable heavy domain and said second pseudo variable heavy domain is N-terminal to said second pseudo variable light domain and/or.

53. The heterodimeric protein composition according to any of claims 48 to 52, wherein the variable heavy chain comprises the amino acid sequence of SEQ ID NO:186 and the variable light domain comprises the amino acid sequence of SEQ ID NO:170.

54. The heterodimeric protein composition of any of claims 48 to 53, wherein the pseudo variable heavy domain comprises the amino acid sequence of SEQ ID NO:190 and the pseudo variable light domain comprises the amino acid sequence of SEQ ID NO:174.

55. The heterodimeric protein composition of any of claims 48 to 53, wherein the pseudo variable heavy domain comprises the amino acid sequence of SEQ ID NO:194 and the pseudo variable light domain comprises the amino acid sequence of SEQ ID NO:178.

56. The heterodimeric protein composition of any of claims 48 to 53, wherein the pseudo variable heavy domain comprises the amino acid sequence of SEQ ID NO:198 and the pseudo variable light domain comprises the amino acid sequence of SEQ ID NO:182.

57. The heterodimeric protein composition of any of claims 48 to 56, wherein the TTA is selected from the group consisting of EGFR, FOLR1, B7H3, EpCAM, Trop2, and CA9.

58. The heterodimeric protein composition of any of claims 48 to 57, wherein said first and second sdABDs bind to the same TTA and/or said third and fourth sdABDs bind to the same TTA.

59. The heterodimeric protein composition according to any of claims 48 to 58, wherein said first, second, third, and fourth sdABDs bind to the same TTA.

60. The heterodimeric protein composition according to any of claims 48 to 59, wherein said first and second sdABD-TTAs are the same and/or said third and fourth sdABD-TTAs are the same.

61. The heterodimeric protein composition according to any of claims 48 to 59, wherein said first and second sdABD-TTAs are different and/or said third and fourth sdABD-TTAs are different.

62. The heterodimeric protein composition according to any of claims 48 to 57, wherein said first, second, third, and fourth sdABDs bind to the different TTAs.

63. The heterodimeric protein composition of any of claims 48 to 62, wherein said sdABD(s) is selected from the group consisting of SEQ ID NOS:50, 54, 58, 62, 66, 70, 74, 78, 82, 86, 90, 94, 98, 102, 106, 110, 114, 118, 122, 126, 130, 134, 138, 142, 146, 150, 154, 158, 162, and 166.

64. The heterodimeric protein composition of any of claims 48 to 63, wherein said first and/or second cleavable linkers are cleaved by a human protease selected from the group consisting of MMP2, MMP9, Cathepsin S, Cathepsin K, Cathespin L, GranzymeB, uPA, Kallekriein7, matriptase, and thrombin

65. The heterodimeric protein composition of any of claims 48 to 64, wherein said first and/or second optional cleavable linkers are cleaved by a human protease selected from the group consisting of MMP2, MMP9, Cathepsin S, Cathepsin K, Cathespin L, GranzymeB, uPA, Kallekriein7, matriptase, and thrombin.

66. The heterodimeric protein composition of any of claims 48 to 65, wherein said first Fc monomer comprises an amino acid sequence selected from the group consisting of Pro584 (SEQ ID NO:44), Pro585 (SEQ ID NO:45), and Pro586 (SEQ ID NO:46).

67. The heterodimeric protein composition of any of claims 48 to 66, wherein said second Fc monomer comprises an amino acid sequence of Pro575 (SEQ ID NO:412 or Pro576 (SEQ ID NO:43).

68. A nucleic acid composition comprising (a) a first nucleic acid encoding said first monomer of any of claims 48 to 67, and/or (b) a second nucleic acid encoding said second monomer of any of claims 48 to 67.

69. An expression vector composition comprising said first nucleic acid according to claim 68, and/or said second nucleic acid according to claim 68.

70. A host cell for expressing said heterodimeric protein composition of any of claims 47 to 67 comprising said expression vector composition according to claim 69.

71. A method of making a heterodimeric protein according to any of claims 48 to 67 comprising: culturing the host cell of claim 70 under conditions to express the heterodimeric protein, and recovering the heterodimeric protein.

72. A method of treating cancer comprising administering the heterodimeric protein of any one of claims 48 to 67.
